(19) United States
(12) Patent Application Publication
(10) Pub. No.: US 2026/0049285 A1
(43) Pub. Date: Feb. 19, 2026

(54) 3D ISLET FORMATION FROM ENDOCRINE PROGENITOR CELLS

(71) Applicant: Spiber Technologies AB, Stockholm (SE)

(72) Inventor: Siqin WU, Stockholm (SE)

(21) Appl. No.: 19/101,672

(22) PCT Filed: Aug. 7, 2023
(86) PCT No.: PCT/EP2023/071814

§ 371 Date: Feb. 6, 2025

(30) Foreign Application Priority Data
Aug. 8, 2022 (EP) 22189347.2

(51) Int. Cl. C12N 5/071; A61K 35/39

(52) U.S. Cl. CPC C12N 5/0676; A61K 35/39; C12N 2501/727; C12N 2506/02; C12N 2513/00; C12N 2533/52

(57) ABSTRACT

The present disclosure relates to a method for the generation of cells of the pancreatic lineage, for example pancreatic islet-like cell aggregates comprising pancreatic β-cells, which method comprises the steps of providing a single cell suspension of a population of endocrine progenitor (EP) cells, allowing said EP cells in single cell suspension to form 3D structures and culturing said cells under conditions permissive of differentiation into pancreatic monohormonal β-cells. The present disclosure also relates to pancreatic islet-like cell aggregates obtainable by said method as well as to medical uses thereof.

### RELATED APPLICATIONS

This application is a 35 U.S.C. § 371 filing of International Patent Application No. PCT/EP2023/071814, filed Aug. 7, 2023, which claims priority to European Patent Application No. 22189347.2, filed Aug. 8, 2022, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Jan. 31, 2025, is named 761711_AWA9-005US_ST26.xml and is 18,453 bytes in size.

### TECHNICAL FIELD

The present disclosure relates to a method for the generation of cells of the pancreatic lineage, for example pancreatic islet-like cell aggregates comprising pancreatic β-cells, which method comprises the steps of providing a single cell suspension of a population of endocrine progenitor (EP) cells, allowing said EP cells in single cell suspension to form 3D structures and culturing said cells under conditions permissive of differentiation into pancreatic monohormonal β-cells. The present disclosure also relates to pancreatic islet-like cell aggregates obtainable by said method as well a medical uses thereof.

### BACKGROUND

Diabetes mellitus is a major worldwide health crisis, affecting more than 200 300 million people worldwide according to the International Diabetes Federation. Type 1 diabetes results from autoimmune destruction of the insulin-producing pancreatic β-cells and type 2 diabetes is characterized by peripheral insulin resistance as well as the inability to produce enough insulin to overcome this resistance. Other, less common forms a diabetes associated with impaired insulin production include gestational diabetes, maturity onset diabetes of the young, neonatal diabetes mellitus and loss of islets in pancreatitis. Patients suffering from type 1 diabetes are treated with injections of exogenous insulin, which provide some level of control over blood glucose levels and has significantly reduced diabetes morbidity, however this is not a curative treatment and is associated with short and long term complications. Thus, although insulin treatment has saved countless diabetics from early death, it represents an ameliorative treatment rather than a cure.

The pancreatic islets, also referred to as islets of Langerhans, are the regions of the pancreas that contain its endocrine cells. The pancreatic islets are arranged in density routes throughout the human pancreas, and are important in the metabolism of glucose. Hormones produced in the pancreatic islets are secreted directly into the blood flow by (at least) five types of cells: α-cells producing glucagon: β-cells producing insulin and amylin; delta cells producing somatostatin; epsilon cells producing ghrelin and PP cells (gamma cells or F cells) producing pancreatic polypeptide. The cytoarchitecture of pancreatic islets is critical for cell-cell communication and coordinated hormone secretion.

Diabetic patients, particularly those suffering from type 1 diabetes, could potentially be cured through transplantation of pancreatic beta cells which produce insulin. Said pancreatic beta cells may be transplanted as pancreatic islets or islet-like structures. Generating an unlimited supply of human beta cells from pluripotent cells could provide therapy to millions of patients. Thus a cure for diabetes could be achieved by replacing lost β-cells in the patient in need thereof. This approach was demonstrated in an animal model several decades ago: rats rendered diabetic by the β-cell toxin streptozotocin could be cured by injection of isogeneic islets (reviewed in Murtaugh 2007). Transplantation of pancreatic progenitors derived from human pluripotent represents a promising way to treat diabetes. However, reliable and safe strategies for obtain the required cell mass are needed and rely on efficient differentiation protocols which may be scaled up to meet therapeutic needs.

Pluripotent cells (PSCs), such as embryonic stem cells (ESCs) and induced pluripotent stem cells (refers to herein as iPS cells or IPCs), have the capacity to differentiate into any somatic cell type, and the possibilities to exploit the therapeutic potential of pluripotent cells have considerable scientific and public interest.

A number of different studies over the past decade have demonstrated that it is possible to generate pancreatic cells, including both polyhormonal and monohormonal insulin-expressing cells from hPSCs (US 2019/0359943; U.S. Pat. No. 10,253,298; US 2011/0280842; Nostro et al., (2015); D'Amour et al., (2006)).

However, the methods of the prior art do not provide pancreatic β-cells with the required efficiency and/or reproducibility for clinical applications. Therefore, alternative methods are needed for more efficient derivation of desired cell types from pluripotent cells. There are major challenges to using stem cells to their full potential, including; (i) developing in vitro differentiation methods that ensure the generation of enriched cell population of specific desired cell types; (ii) ensuring the identity and functionality of in vitro generated cells; and (iii) eliminating contaminating non-desired cell types that may disrupt the functions of the desired cell type. The presence of undesired cell types, including for example polyhormonal pancreatic cells, in cultures may for example impose safety issues in prospect replacement therapies or may negatively influence the outcome of drug screening or disease modeling.

Thus, as is evident from the different sections of this background description, the provision of differentiation strategies that overcome said drawbacks is desirable. Thus, it is desirable to provide differentiation strategies for efficiently obtaining pancreatic islet-like cell aggregates (or islet-like structures) of high quality in vitro in a reproducible manner.

### SUMMARY OF THE INVENTION

It is an object of the present disclosure to provide a differentiation strategy for generation of pancreatic islet-like cell aggregates in vitro, which strategy overcomes and/or alleviates the above mentioned or other drawbacks of current strategies.

It is an object of the present disclosure to provide an efficient and reproducible in vitro differentiation protocol for production of the pancreatic islet-like cell aggregates, which islet-like cell aggregates exhibit the desired ability to produce insulin as a response to glucose stimulation.

It is an object of the present disclosure to provide an efficient and reproducible in vitro differentiation protocol that allows for the production of pancreatic islet-like cell aggregates, in large numbers.

It is furthermore an object of the present disclosure to provide an in vitro differentiation protocol, which allows for obtaining cultures of pancreatic islet-like cell aggregates exhibiting a high percentage of pancreatic monohormonal β-cells, and a low percentage of contaminating cell types. Said pancreatic islet-like cell aggregates also exhibit monohormonal α-cells.

It is also an object of the present disclosure to provide pancreatic islet-like cell aggregates, wherein a high percentage of cells exhibit functional characteristics of cells of the pancreatic β-cells lineage.

It is also an object of the present disclosure to provide pancreatic islet-like cell aggregates exhibiting a high percentage pancreatic monohormonal β-cell, and a low percentage of contaminating cell types. Said pancreatic islet-like cell aggregates also exhibit monohormonal α-cells. In particular, it is important that the desired cells are viable and healthy. Such islet-like cell aggregates, or cells obtainable therefrom, may be useful for a number of applications, including therapeutic and scientific/biotechnology applications, for example in vitro drug development and screening. For example, such islet-like cell aggregates or cells could be used for cell transplantation (in other words cell replacement therapy) into patients in need thereof.

These and other objects, which are evident to the skilled person from the present disclosure, are met by different aspects of the invention as claimed in the appended claims and as generally disclosed herein.

The methods disclosed herein for generation of pancreatic islet-like cell aggregates and/or cells obtainable from said aggregates, from pluripotent cells involves the use of specific culture conditions, for example combination of soluble factors and environmental conditions and timing, which direct differentiation of a remarkably high proportion of pluripotent cells into cells the desired cell fate.

The present disclosure is based on the surprising realization that providing of stage 5 endocrine progenitor (EP) cells (also referred to herein as endocrine precursor cells) in single cells suspension and allowing said population of EP cells to form 3D structures and continuing culturing said population of EP cell in the form of 3D structures in 3D culture conditions permissive of differentiation into pancreatic monohormonal β-cells to provide pancreatic islet-like cell aggregates, leads to unexpectedly high quality of pancreatic islet-like cell aggregates. Said islet-like cell aggregates comprise an unexpectedly high percentage of monohormonal β-cells, low percentage of polyhormonal cells, low percentage of monohormonal alpha cells (α-cells) and/or delta cells and low percentage of proliferating cells. The present inventors have found that culturing cells of the pancreatic lineage on a 2D substrate, such as adherent cultures on a 2D substrate, until the developmental stage of endocrine progenitor (EP) cells is reached, and dissociating the cells, such as dissociating to into single cell suspension, and thereafter allowing the cells to form 3D structures, leads to the generation high quality of pancreatic islet-like cell aggregates. Importantly, as is shown in the appended Examples, the timing of the transfer from 2D culture conditions to 3D culture conditions is of great importance. Without being bound by theory, it appears that timing said transfer of cells influences the development/differentiation of EP cells into high quality pancreatic islet cells. Importantly, the EP cells in single cell suspension which are allowed to form 3D structures according to the method as disclosed herein have the potential to develop into mature and functional pancreatic β-cells.

Thus, in a first aspect of the present invention there is provided a method for the generation pancreatic islet-like cell aggregates in vitro, comprising the steps of
i) providing a population of endocrine progenitor (EP) cells, such as EP cells characterized by the expression of NEUROD1; such as EP cells characterized by the expression of NKX6.1 and NEUROD1;
ii) providing a single cell suspension of said population of EP cells;
iii) allowing said population of EP cells in single cell suspension to form 3D structures;
iv) culturing said population of EP cell in the form of 3D structures in 3D culture conditions permissive of differentiation into pancreatic monohormonal β-cells to provide pancreatic islet-like cell aggregates; and
v) thereby generating pancreatic islet-like cell aggregates comprising monohormonal β-cells,
wherein said pancreatic islet-like cell aggregates comprise at least approximately 25% monohormonal β-cells.

As used herein, the term “pancreatic islet-like cell aggregates” or “islet-like aggregates” refers to cell aggregates of pancreatic cells, which show characteristics of pancreatic islets in vivo. In particular said aggregates exhibit desirable properties of high number of monohormonal β-cells, a desired number of monohormonal α-cells, low number of polyhormonal cells (including low number of polyhormonal β-cells and low number of polyhormonal α-cells), low number of non-endocrine cells and low number of proliferating cells. In particular, it is highly desirable that the pancreatic islet-like cell aggregates obtained in vitro mimic the characteristics of pancreatic islets in vivo, both in terms of distribution of cell types present herein and also in their functional properties.

The skilled person appreciates that the herein described percentages relate to average percentages exhibited by the pancreatic islet-like cell aggregates. Thus for example, 100 aggregates are analyzed, the average number of said cell types is as indicated herein. Thus, step v) may be reworded as “thereby generating population of pancreatic islet-like cell aggregates comprising monohormonal β-cells, wherein said population comprises pancreatic islet-like cell aggregates which comprise at least 25% monohormonal β-cells.” In this context, the below recited properties refer to the average percentages in the population of the pancreatic islet-like cell aggregates according to the present disclosure.

Thus, in one embodiment said pancreatic islet-like cell aggregates comprise, or said population comprises pancreatic islet-like cell aggregates which comprise, at least approximately 25%, such as at least approximately 30%, such as at least approximately 35%, such as at least approximately 40%, such as at least approximately 45%, such as at least approximately 50%, such as at least approximately 55%, such as at least approximately 60%, such as at least approximately 65%, such as at least approximately 70% monohormonal β-cells. In one embodiment said pancreatic islet-like cell aggregates comprise, or said population comprises pancreatic islet-like cell aggregates which comprise, approximately from 25 to 70%, such as from 30 to 70%, such as from 30 to 70% monohormonal β-cells, such as from 35 to 70%, from 35 to 70%, such as from 40 to 70%, such as from 45 to 70%, such as from 45 to 65%, such as from 45 to 60%, such as from 45 to 55%, such as approximately 50% monohormonal β-cells. In one embodiment said pancreatic islet-like cell aggregates comprise, or said population comprises pancreatic islet-like cell aggregates which comprise, approximately from 35 to 65%, such as from 40 to 65%, such as from 40 to 60% monohormonal β-cells.

In one embodiment, said pancreatic islet-like cell aggregates comprise, or said population comprises pancreatic islet-like cell aggregates which comprise, approximately from 7 to 25%, such as from 7 to 20%, from 10 to 20%, such as from 15 to 20%, such as approximately 20% monohormonal α-cells.

In one embodiment said pancreatic islet-like cell aggregates comprise, or said population comprises pancreatic islet-like cell aggregates which comprise, at most approximately 10%, such as at most approximately 7%, such as at most approximately 6%, such as at most approximately 5%, such as at most approximately 4%, such as at most approximately 3%, such as at most approximately 2%, such as at most approximately 0.5%, such as at most approximately 0.3%, such as at most approximately 0.1%, poly hormonal α-cells.

In one embodiment said pancreatic islet-like cell aggregates comprise, or said population comprises pancreatic islet-like cell aggregates which comprise, at most approximately 10%, such as at most approximately 7%, such as at most approximately 6%, such as at most approximately 5%, such as at most approximately 4%, such as at most approximately 3%, such as at most approximately 2%, such as at most approximately 0.5%, such as at most approximately 0.3%, such as at most approximately 0.1%, polyhormonal β-cells.

In one embodiment, said pancreatic islet-like cell aggregates comprise, or said population comprises pancreatic islet-like cell aggregates which comprise, less than 5%, such as less than 4%, such as less than 3%, such as less than 1%, such as less than 1% delta cells.

In one embodiment said pancreatic islet-like cell aggregates comprise, or said population comprises pancreatic islet-like cell aggregates which comprise, at most approximately 5, such as at most approximately 4%, such as at most approximately 3%, such as at most approximately 2%, such as at most approximately 1%, such as at most approximately 0.5%, such as at most approximately 0.1%, proliferating cells, such as proliferating cells which express Ki-67.

In one said pancreatic islet-like cell aggregates comprise, or said population comprises pancreatic islet-like cell aggregates which comprise, at least 40%, such as at least 50% monohormonal β-cells; approximately from 15 to 20%, such as approximately 20% monohormonal α-cells and less than approximately 2%, such as less than approximately 1%, proliferating cells. In one embodiment, the composition of the pancreatic islet-like cell aggregates is scored, in other words investigated, at day 38-42 of culture or later. For example on day 38, 39, 40, 41 or 42. In other words, the composition of the pancreatic islet-like cell aggregates is scored, in other words investigated, at the end of Stage 6.

The pancreatic islets, also known as islets of Langerhans are the regions of the pancreas that contain its endocrine (hormone-producing) cells. There are about 1 million islets distributed throughout the pancreas of a healthy adult human, each of which measures an average of about 0.2 mm in diameter. Each islet is separated from the surrounding pancreatic tissue by a thin fibrous connective tissue capsule which is continuous with the fibrous connective tissue that is interwoven throughout the rest of the pancreas.

Hormones produced in the pancreatic islets are secreted directly into the blood flow by (at least) five types of cells. The endocrine cell types in islets include: alpha cells producing glucagon, beta cells producing insulin and amylin, PP cells (gamma cells or F cells) producing pancreatic polypeptide, delta cells producing somatostatin and epsilon cells producing ghrelin. In humans, beta cells correspond to about 40-50% of the cells. In addition to endocrine cells, there are stromal cells (fibroblasts), vascular cells (endothelial cells, pericytes), immune cells (granulocytes, lymphocytes, macrophages, dendritic cells) and neural cells. The skilled person is familiar with the composition and cytoarchitecture of pancreatic islets.

The endocrine progenitor (EP) cells may be scored by the expression of the specific marker NEUROD1, which during the development along the pancreatic endocrine lineage is upregulated as the cells adopt the developmental EP cell stage (also referred to as stage 5, see FIG. 1A). Cells of earlier developmental stage 4 (pancreatic progenitor cells) do not express NEUROD1. The EP cells may also be scored as double positive for NKX6.1 and at least one marker not expressed by the pancreatic progenitor cell, for example double positive for NKX6.1 and NEUROD1; or NKX6.1 and NGN3. As explained below, other markers and combinations thereof may be used to identify EP cells. Pancreatic endocrine progenitor cells express at least one, or two or three or all four, of the following markers: PDX1, NKX6.1, NGN3 and NEUROD1.

Thus, in one embodiment of the method as disclosed herein, the cell population of EP cells provided in step i) is characterized by expression of NEUROD1 and NKX6.1. In another embodiment, the cell population of EP cells provided in step i) is characterized by expression of NKX6.1 and NGN3 or by the expression of NEUROD1 and NGN3. Alternatively, the EP cells may also be identified by the expression of PDX1, NKX6.1 and NGN3: or PDX1, NKX6.1 and NEUROD1; or PDX1, NKX6.1, NGN3 and NEUROD1.

The skilled person will appreciate that step ii) of providing a single cell suspension of the population of EP cells involves dissociation of EP cells from adherent culture on a 2D substrate into single cells. Such dissociation may involve the use of dissociation reagents, such as naturally occurring enzymes, gentler non-enzymatic alternatives, or may work by chelating calcium to prevent cadherins from attaching, releasing cells from surfaces and one another. Dissociation may be performed by mechanical means. Non-limiting examples include of dissociation reagents include trypsin, collagenases, displases as well as dissociation reagents such as Accutase®, Accumax™ and ACS-3010.

In embodiment, step ii) of providing a single cell suspension of the population of EP cells involves dissociation of EP cells from adherent culture on a 2D substrate into single cells. In one embodiment, step ii) of providing a single cell suspension of the population of EP cells involves dissociation of EP cells by enzymatic means, such as using a solution comprising enzymes, for example proteolytic and (pr collagenolytic enzymes. For example, such a solution may be Accutase®. The skilled person is aware of the appropriate methods for dissociation of EP cells and the provision of a single cell suspension of the population of EP cells.

In one embodiment, step ii) and step iii) are performed prior to subjecting the cells to conditions permissive of differentiation into pancreatic monohormonal β-cells, such as prior to culturing the cells in a medium permissive of differentiation into pancreatic monohormonal β-cells.

In one embodiment, said dissociation is performed prior to subjecting the cells to conditions permissive of differentiation into pancreatic monohormonal β-cells, such as prior to culturing the cells in a medium permissive of differentiation into pancreatic monohormonal β-cells. In one embodiment, said dissociation is performed at most 96 hours, such as at most 72 hours, such as at most 48 hours such as at most 24 hours after changing from conditions permissive of differentiation into endocrine progenitor cells to conditions permissive of differentiation into pancreatic monohormonal β-cells, such as after changing culture medium from a medium permissive of differentiation into endocrine progenitor cells to a medium permissive of differentiation into pancreatic monohormonal β-cells. For example at most 96 hours, such as at most 72 hours, such as at most 48 hours such as at most 24 hours hours after changing from S5 to S6 medium as described in the appended Examples. In one embodiment, said dissociation is performed 24-48 hours after changing from conditions permissive of differentiation into endocrine progenitor cells to conditions permissive of differentiation into pancreatic monohormonal β-cells.

Thus, in one embodiment, steps i), ii) and iii) are performed in conditions permissive of differentiation into endocrine progenitor cells and step iv) is performed in conditions permissive of differentiation into pancreatic monohormonal β-cells.

In step iii) the EP cells in single cell suspension are allowed to form 3D structures. In one embodiment, 3D culture conditions allow for self-aggregation of the cells. Said aggregation may be forces, or induced aggregation but may also be spontaneous aggregation.

In one embodiment, step iii) is performed in the presence of a ROCK inhibitor. Said ROCK inhibitor may be H1152 or any analog or agonist thereof. In one embodiment, the concentration of H1152 is in the range of >0 to 10 μM. As shown in the present Examples, and without being bound by theory, the inventors consider that H1152 may promote survival of S5 EP cells as single cells in suspension.

Thus, in one embodiment of the method as disclosed herein, said ROCK inhibitor is present in the culture medium during approximately 24 hour during stage iii).

Without being bound by theory, it is envisioned that self-aggregation allows for selective enrichment of endocrine progenitor cells. As shown in the appended Examples formation of 3D structures, also referred to as aggregation, leads to selective enrichment and results in increased generation of pancreatic monohormonal β-cells in said cultures. As explained above, said aggregates exhibit desirable properties of high number of monohormonal β-cells, a desired number of monohormonal α-cells, low number of polyhormonal cells (including low number of polyhormonal β-cells and low number of polyhormonal α-cells) and low number of proliferating cells. In one embodiment, said pancreatic monohormonal β-cells are generated as part of cell aggregates. In one embodiment, said aggregates comprise monohormonal β-cells. In one embodiment said aggregates further comprise pancreatic monohormonal alpha cells. In one embodiment said aggregates further comprise pancreatic monohormonal delta cells, such as less than 3%, such as less than 2%, such as less than 1% delta cells. The skilled person appreciates that the percentages here recited are to be interpreted as related to average percentages exhibited by the pancreatic islet-like cell aggregate. Said percentages may be evaluated for said pancreatic islet-like cell aggregates as such or for said populations comprising pancreatic islet-like cell aggregates. For clarity, said cell aggregates are herein referred to as pancreatic islet-like cell aggregates and are obtainable according to the method as defined herein.

As used herein, the term “differentiation” refers to the process by which an unspecialized (“uncommitted”) or less specialized (“less committed”) cell acquires the features of a specialized cell (“more committed”) such as, for example, a pancreatic cell. A differentiated cell is one that has taken on a more specialized (“committed”) position within the lineage of a cell. As used herein, the term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

As used herein, the term “lineage” of a cell refers to the heredity of the cell, i.e., which cells it came from and to what cells it can give rise. The lineage of a cell places the cell within a hereditary scheme of development and/or differentiation in vivo or in vitro.

As used herein, the term “lineage-specific marker” refers to a characteristic specifically associated with the phenotype of cells of a lineage of interest and can be used to assess the differentiation of an uncommitted cell to the lineage of interest.

The person skilled in the art is well aware of the different developmental stages of pancreatic endocrine lineage, such as of the pancreatic β-cell lineage.

As used herein, the term “pancreatic β-cell lineage” refers to the hereditary scheme of development and/or differentiation in vivo or in vitro ultimately leading to the provision of cells which exhibit properties of characteristic of pancreatic monohormonal β-cells, such as production of insulin and expression of at least one of PDX1, NKX6.1 and NEUROD1. The skilled person will appreciate the cells of the pancreatic β-cell lineage may be any cells of earlier developmental stages of said cells.

As used herein, the term “precursor thereof” relates to a cell of the pancreatic lineage, such as precursor of pancreatic β-cell, and refers to any cell that is capable of differentiating into a pancreatic β-cell, including for example, a pluripotent stem cell, a definitive endoderm cell, a primitive gut tube cell, a posterior foregut cell, pancreatic progenitor cell or endocrine progenitor cell when cultured under conditions suitable for and/or permissive of differentiation of the precursor cell into the pancreatic lineage, such as precursor of pancreatic β-cell.

The differentiation of cells along the pancreatic β-cell lineage comprises the differentiation of less committed to more committed cell types. Briefly, the development of insulin-producing pancreatic β-cells represents the culmination of a complex developmental program and involves the in vivo steps of cells of the posterior foregut assuming a pancreatic identity, expanding pancreatic primordia adopting an endocrine fate, and a subset of these precursors becoming competent to generate β-cells. Factors, for example transcription factors, which have been shown to be important in the development of cells of the pancreatic β-cell lineage include amongst other PDX1 (pancreatic and duodenal homeobox 1), PTF1A (pancreas specific transcription factor 1a, NGN3 (Neurogenin 3), NEUROD1 (Neurogenic differentiation 1) and NKX6.1 (Homeobox protein NKX-6.1). This list of factors is to be viewed as non-limiting and additional factors will be discussed below. Additionally, the development of cells along the pancreatic β-cell lineage requires signaling from extrinsic factors, such as, but not limited to, transforming growth factor-β (TGFβ) and retinoic acid (RA). Recent studies suggest that TGFβ signaling induces definitive endoderm in mouse and human embryonic stem (ES) cells, and that RA treatment promotes PDX1 expression and pancreas specification in ES cell-derived endoderm. The skilled person appreciates that the in vitro differentiation of cells of the pancreatic β-cell lineage requires the addition of extrinsic factors to the cell growth media at the appropriate stage of the differentiation process and in the suitable/permissive concentrations, which in principle mimic the in vivo development of said cells.

Thus, the skilled person realizes that “conditions permissive of differentiation” to a recited cell type refers to conditions which allow cells to exhibit the characteristics of said cell type and may include combinations of cell culture media, presence and/or absence of extrinsic factors as well as timing thereof. Below follows a short summary of the developmental stages of pancreatic development. The skilled person appreciates that said development can be described in developmental stages. Each developmental stage 0-6 is characterized by the expression of a set of factors (often referred to as markers).

The skilled person appreciates that the process of differentiating pluripotent stem cells into functional pancreatic endocrine cells, such as monohormonal pancreatic β-cells in vitro may be viewed in some aspects as progressing through six consecutive stages, as is shown in the schematic illustration shown in FIGS. 1A-1B, which stages correspond to the developmental stages in vivo. The skilled person is well familiar with said developmental stages in vivo and said stages are considered to be encompassed by the common general knowledge in the field.

In this step-wise progression, Stage 0 refers to undifferentiated pluripotent cells, such hES cells or iPS cells; stage 1 refers to cells expressing markers characteristic of definitive endoderm (DE) cells; stage 2 refers to cells expressing markers characteristic of primitive gut tube cells (PGT); stage 3 cells expressing markers characteristic of posterior foregut (PF) cells; stage 4 refers to cells expression markers characteristic of pancreatic progenitor (PP) cells; stage 5 refers to cells expressing markers characteristic of pancreatic endocrine progenitor (EP) cells; stage 6 refers to cells expressing markers characteristic of endocrine islet cells, such as pancreatic β-cells. Stage 6 cells, as defined herein, may form pancreatic islet-like cell aggregates in vitro (cell aggregates in vitro) which mimic the pancreatic islets found in vivo. The skilled person appreciates that not all cells in a particular population progress through these stages at the same rate, i.e., some cells may have progressed less, or more, down the differentiation pathway than the majority of cells present in the population.

Below follows a non-exhaustive description of the characteristics associated with cells in different stages of in vitro culture as described above. The skilled person will appreciate that the different stages of in vitro culture correspond to developmental stages in vivo. The skilled person will appreciate that by selectively choosing which proteins to monitor the expression of, one may follow the developmental progress along the pancreatic endocrine lineage, such as the pancreatic β-cell lineage. When needed, the expression of more than one protein characteristic for a developmental stage may be evaluated. As cells progress through development, expression of certain proteins is up- and down-regulated which make said proteins suitable as markers of different developmental stages.

The term “definitive endoderm cells” as used herein, refers to cells which exhibit the characteristics of cells which have arisen from the epiblast during gastrulation and which form the gastrointestinal tract and its derivatives. Definitive endoderm cells express at least one, or two or all three, of the following markers: CXCR4, FOXA2 and SOX17. Thus, definitive endoderm cells may be identified by the expression of at least one, or two or all three, of the following markers: CXCR4, FOXA2 and SOX17. In particular, definitive endoderm cells may be identified by the expression of SOX17.

The term “primitive gut tube cells” as used herein, refers to cells derived from definitive endoderm that can give rise to all endodermal organs, such as lungs, liver, pancreas, stomach, and intestine. Primitive gut tube cells express at least one, or two, of the following markers: HNF1β and HNF4α. Thus, primitive gut tube cells may be identified by the expression of HNF1β, HNF4α or of both HNF1β and HNF4α.

The term “posterior foregut cells” as used herein, refers to endoderm cells that give rise to the stomach, liver, pancreas, gall bladder, and a portion of the duodenum. Posterior foregut cells express PDX1 or both PDX1 and HNF6. Thus, posterior foregut cells may be identified by the expression of PDX1, HNF6 or both PDX1 and HNF6.

The term “pancreatic progenitor cells” as used herein, refers to cells that express at least one, or two or three or four or five or all six, of the following markers: PDX1, PTF1A, NKX6.1, SOX9, CPA and HNF6. As illustrated in FIGS. 1A-1B, pancreatic progenitor cells express PDX1, NKX6.1, PTF1A and SOX9. In particular, pancreatic progenitor cells co-express PDX1 and NKX6.1.

Thus, pancreatic progenitor cells may be identified by the expression of PDX1 and NKX6.1. Pancreatic progenitor cells may be identified by the expression of PDX1, NKX6.1 and one or both of PTF1A and SOX9.

The term “endocrine progenitor cells” as used herein refer to pancreatic endoderm cells capable of becoming a pancreatic hormone expressing cell. Pancreatic endocrine progenitor cells express at least one, or two or three or all four, of the following markers: PDX1, NKX6.1, NGN3 and NEUROD1. Endocrine progenitor cells may be identified by the expression of NXK6.1 and NEUROD1. In particular, endocrine progenitor cells may be distinguished from pancreatic progenitor cells by their expression of NEUROD1 and NGN3, which are both not expressed by pancreatic progenitor cells. Endocrine progenitor cells may also be identified by the expression of PDX1, NKX6.1 and NGN3: or PDX1, NKX6.1 and NEUROD1; or PDX1, NKX6.1, NGN3 and NEUROD1.

The term “pancreatic islet cells,” as used herein, refer to cells capable of expressing at least one of the following hormones: insulin, glucagon, somatostatin, ghrelin, and pancreatic polypeptide. In addition to these hormones, markers characteristic of pancreatic endocrine cells include one, or two or all three, of PDX1, NKX6.1 and NEUROD1. NEUROD1 is expressed in most, such as essentially all, endocrine islet cells, while PDX1 and NKX6.1 are specific for pancreatic β-cells at stage 6. As used herein, the term “pancreatic islet-like cell aggregates” or “islet-like aggregates” refers to cell aggregates of pancreatic cells, which show characteristics of pancreatic islets in vivo. In particular said aggregates exhibit the properties as discussed above.

In particular, as used herein the term “pancreatic β-cells” or “monohormonal pancreatic β-cell” refers to cells that express insulin, but do not express glucagon or somatostatin. The terms “pancreatic β-cells” and “monohormonal pancreatic β-cell” are used interchangeably herein. Thus, pancreatic monohormonal β-cells may be identified by the expression of insulin and the lack of expression of glucagon and/or somatostatin.

Pancreatic β-cells are monohormonal cells, in other words express only one hormone which is insulin. In contrast, polyhormonal in the contexts of pancreatic cells express more than one hormone, such as at least two of insulin, glucagon and somatostatin.

For example, as illustrated in FIGS. 1A-1B, progress through the developmental stages discussed above can be followed by marker expression. For example, the progression from stage 0 to stage 1 is associated with the downregulation of expression of OCT4, NANOG and SOX2 and upregulation of expression of SOX17, FOXA and CXCR4. The progression to from stage 1 to stage 2 is associated with upregulation of expression of HNF1β and HNF4α. The progression to from stage 2 to stage 3 is associated with upregulation of expression of PDX1 and HNF6.

The progression from stage 3 to stage 4 is associated with maintainance of expression of PDX1 and HNF6, and upregulation of expression of NKX6.1, PTF1A and SOX9. The progression to from stage 4 to stage 5 is associated with the downregulation of expression of PTF1A and SOX9, maintainance of expression of PDX1 and NKX6.1 and upregulation of expression of NGN3 and NEUROD1. The progression from stage 5 to stage 6 pancreatic β-cells is associated with the downregulation of expression of NGN3, maintainance of expression of PDX1, NKX6.1 and NEUROD1 and upregulation of expression of insulin and C-peptide. Thus, for example the upregulation of NKX6.1 in PDX1+ cells marks the progression to stage 4.

The skilled person will appreciate that the progression of development of cells ot the pancreatic lineage may be further divided into additional stages, for example based on level of expression of markers. As an example, in a review article by Verhoeff and coworkers (Stem Cell Reviews and Reports (2022): 18, 2683-2698), seven stages of differentiation into islet-like clusters are summarized, wherein stages 1˜4 and 7 correspond to stages 1˜4 and 6 as used in the present application. Stage 5 according to Verhoeff is characterized by expression of NKX6.1 and low expression of NGN3, while stage 6 according to Verhoeff is characterized by high expression of NGN3 and expression of endocrine hormones. Stage 5 as defined in the present disclosure corresponds Verhoeff's stages 5 and 6.

Cells generated are identified or characterized by phenotypic characteristics, morphological characteristics and/or expression of cell markers, which are readily appreciated by those of skill in the art of evaluating such cells. The term “markers”, as used herein, refers to nucleic acid or polypeptide molecules that are differentially expressed in cells of interest. Non-limiting examples of markers of the β-cell lineage discussed in the present disclosure include: CXCR4, FOXA2, SOX17, HNF1β, HNF4α, PDX1, HNF6, PDX1, PTF1A, NKX6.1, SOX9, NGN3, NEUROD1 and insulin. As explained above, combinations of said markers are characteristic for different developmental stages along the β-cell lineage. As discussed above, the skilled person will appreciate that it is possible to select markers such that the expression or lack of expression of combinations of markers allows to distinguish between cells of different developmental stages along the pancreatic endocrine lineage (see FIGS. 1A-1B). The present inventors have used expression of different markers to distinguish between cells of different developmental stages as exemplified in the appended examples. As used herein, the term “characterized by expression of” when referring to cells of a cell populations is to be interpreted as related to the expression of a given marker or a set of markers. Conversely, the term “characterized by the lack of expression of” or “lack expression of” or “do not express” when referring to a cell of a cell population is to be interpreted as related to the absence of expression of a given marker or set of markers. The skilled person is well familiar with the use of marker expression as a way to distinguish between cells with different characteristics, such as cells of different developmental stages of the pancreatic β-cells lineage.

It will be appreciated by the skilled person that when a cell population is analyzed by a method which is limited in terms of the number of markers that can be scored simultaneously, for example due to limitation of the method as such or limitations due to the reagents used, it is possible to select a subset of markers which allow for distinguishing between a first and a second population of cells. As an example, a cell population of pancreatic progenitor cells characterized by expression of PDX1 and NKX6.1 can be distinguished from a population of endocrine progenitor cells characterized by the expression of PDX1, NKX6.1, NEUROD1 and NGN3. For example, pancreatic progenitor cells as positive for PDX1 or NKX6.1 and negative for one of NGN3 or NEUROD1. The EP cells may be scored as double positive for NKX6.1 and at least one marker not expressed by the pancreatic progenitor cell, for example double positive for NKX6.1 and NEUROD1; or NKX6.1 and NGN3. This principle is applied in the Example section to distinguish between cells with different characteristics. Importantly, said cells if they were to be scored by the other above listed markers would exhibit the full marker profile of the particular population. The fact that only a subset of markers is used in the experimental set up is in no way to be interpreted as representing the lack of expression of the remaining characteristic markers. The skilled person will appreciate that marker expression may be evaluated at nucleotide level, for example mRNA level, or a protein level. Well known methods of evaluating marker expression include, but are not limited to immunohistochemistry, in situ hybridization, FACS, RNA-sequencing, use of arrays, such as microarrays, as well as quantitative PCR. The skilled person is aware of these and other suitable methods.

In this context, differential expression means an increased level for a positive marker and a decreased level for a negative marker as compared to an undifferentiated cell or a cell in a different developmental stage. The detectable level of the marker nucleic acid or polypeptide is sufficiently higher or lower in the cells of interest compared to other cells, such that the cell of interest can be identified and distinguished from other cells using any of a variety of methods known in the art.

As used herein, a cell is “positive for” a specific marker, or “positive”, when the specific marker is sufficiently detected in the cell, in other word expressed by the cell. Thus, a cell which is characterized by the expression of a given marker, is positive for said marker. Conversely, the cell is “negative for” a specific marker, or “negative”, when the specific marker is not sufficiently detected in the cell. Thus, a cell which is characterized by the lack of expression of a given marker, is negative for said marker. The use of “+” or “−” signs in connection with a marker is herein meant to be understood as positive or negative for said marker (for example NKX6.1⁺ cells are positive for the marker NKX6.1).

Using any method for in vitro differentiation, the ability to obtain a population comprising cells that exhibit the same or similar properties at a given time point is of crucial importance, given that cells at different developmental, maturation or functional stages may respond differently to external and internal cues. To clarify, a factor X may during development lead to the specification of cell types A and B, while treatment with the same factor X may lead to selective cell death of mature cell type A. Hence, it is not only important to know the downstream functional outcome of subjecting a cell to a given factor, it is equally important to subject the cell to said factor during a time window in which said cell is competent to respond to said factor in a desired way. Thus, the skilled person will appreciate the importance of obtaining a synchronous population of cells in order to assure the desired response over the population of cells as a whole. It may, for example, be desirable that a majority of cells in culture are progenitor cells at a given time point.

Research in tissue engineering, stem cells and molecular biology primarily involves cultures of cells on flat plastic or glass dishes/cell culture plates. These cultures are considered adherent cultures. This technique is known as two-dimensional (2D) cell culture. As used herein, the term “2D” in the context of cell culture refers culture on flat cell culture plates, where the cells are adherent directly or indirectly, for example via a cell culture substrate, to said culture plates. Plates may be coated with a cell culture substrate. There are two main categories of cell culture substrates: naturally-occurring and synthetic. The most commonly used natural substrates are collagen, fibronectin, and laminin which make up part of the extracellular matrix. Cells interact with these matrix components via cell surface receptors, such as integrins. Receptors bind to domains of collagen, fibronectin and laminin and trigger intracellular signaling pathways that facilitate adhesion complex formation, and may also trigger cell proliferation or differentiation. The most common synthetic substrate for 2D culture includes poly-lysine, a polymer of lysine containing a positively charged amino group. Substrate choice can have an large impact on how cell growth and attachment and thus is an important parameter to consider.

In one embodiment of the method as disclosed herein, the population of EP cells in step i) is an adherent culture of EP cells on a 2D substrate. In one embodiment of the method as disclosed herein, the cells are cultured on a 2D substrate. In one embodiment, said cells are adherent to said 2D substrate. Said 2D substrate may comprises one or more components selected from the group consisting of laminins (LN) and fragments thereof, vitronectin and fragments thereof, fibronectin and fragments thereof, collagen and fragments thereof, gelatin and fragments thereof, functionalized silk (FN silk) and Matrigel™, such as may comprise one or more components selected from the group consisting of laminins (LN) and fragments thereof, vitronectin and fragments thereof, fibronectin and fragments thereof, collagen, gelatin and Matrigel™, such as may comprise one or more components selected from the group consisting of laminins (LN) and fragments thereof, vitronectin and fragments thereof, fibronectin and fragments thereof and Matrigel™, such as may comprise one or more one or more components selected from the group consisting of laminins (LN) and fragments thereof, vitronectin and fragments thereof, fibronectin and fragments thereof and Matrigel™. In particular, said fragments may comprise functional domains of the proteins. Thus, the skilled person will appreciate that the fragments may correspond to functional domains or comprise functional domains, for example comprise functional domains and further comprise additional N- and/or C-terminal amino acids. Matrigel™ is derived from mouse Engelbreth-Holm-Swarm tumours and contains many unknown components. Laminin is known to be the main component of Matrigel. It may be beneficial, in particular in relation to cultures of cells for therapeutic use, to avoid any animal derived products in addition to using defined medium, in order to achieve a defined and reproducible product and avoid any potential patient safety issues. As used herein, functionalized silk refers to a recombinant fusion protein comprising a silk protein and a cell binding motif. In certain embodiments, said functionalized silk comprises a cell-binding motif selected from RGD, IKVAV (SEQ ID NO: 1), YIGSR (SEQ ID NO: 2), EPDIM (SEQ ID NO: 3), NKDIL (SEQ ID NO: 4), GRKRK (SEQ ID NO: 5), KYGAASIKVAVSADR (SEQ ID NO: 6), NGEPRGDTYRAY (SEQ ID NO: 7), PQVTRGDVFTM (SEQ ID NO: 8), AVTGRGDSPASS (SEQ ID NO: 9), TGRGDSPA (SEQ ID NO: 10), CTGRGDSPAC (SEQ ID NO: 11) and C₁X₁X₂RGDX3X₄X₅C₂ (SEQ ID NO: 12), preferably selected from C₁X₁X₂RGDX3X₄X₅C₂, GRKRK, IKVAV, RGD and CTGRGDSPAC, wherein each of X₁, X₂, X₃, X₄ and X₅ are independently selected from natural amino acid residues other than cysteine; and C₁ and C₂ are connected via a disulphide bond.

In one particular embodiment said functionalized silk comprises a spidroin fragment and a cell binding motif defined as comprising the amino acid sequence C₁X₁X₂RGDX3X₄X₅C₂ (SEQ ID NO:12), wherein each of X₁, X₂, X₃, X₄ and X₅ are independently selected from natural amino acid residues other than cysteine; and C₁ and C₂ are connected via a disulphide bond.

Functionalized silk has been described in WO 2016/207281 and WO 2017/137611 and the disclosures are encompassed herein in their entirety. In particular, said functionalized silk may be a recombinant polypeptide comprising the amino acid sequence
C₁X₁X₂RGDX3X₄X₅C₂ (SEQ ID NO:12), wherein
X₁ is S or T;
X₂ is G, A or V;
X₃ is S or T;
X₄ is G, A, V or P; and
X₅ is G, A or V; and
C₁ and C₂ are connected via a disulphide bond;
and wherein the spidroin fragment is comprising the protein moieties REP and CT, wherein REP is a repetitive fragment of from 70 to 300 amino acid residues, selected from the group consisting of L(AG)nL, L(AG)nAL, L(GA)nL, and L(GA)nGL, wherein n is an integer from 2 to 10;
each individual A segment is an amino acid sequence of from 8 to 18 amino acid residues, wherein from 0 to 3 of the amino acid residues are not Ala, and the remaining amino acid residues are Ala; each individual G segment is an amino acid sequence of from 12 to 30 amino acid residues, wherein at least 40% of the amino acid 25 residues are Gly; and
each individual L segment is a linker amino acid sequence of from 0 to 30 amino acid residues; and
CT is a fragment of from 70 to 120 amino acid residues, having at least 70% identity to
SRLSSPSAVSRVSSAVSSLVSNGQVNMAALPNIISNISSSVSASAPGASGCE VIVQALLEVITALVQIVSSSSVGYINPSAVNQITNVVANAMAQVMG (SEQ ID NO: 13). In one embodiment, the CT fragment has at least 70%, such as at least 80%, such as at least 85%, preferably at least 90%, such as at least 95%, identity to SEQ ID NO: 13. In certain embodiments, the spidroin fragment has at least 70%, such as at least 80%, such as at least 85%, preferably at least 90%, such as at least 95%, identity to SEQ ID NO: 16 or to amino acid residues 18-277 of SEQ ID NO:14. In particular embodiments, said cell-binding motif comprises the amino acid sequence CTGRGDSPAC (SEQ ID NO:11).

In particular embodiments, said functionalized silk comprises an amino acid sequence selected from the group consisting of SEQ ID NO:14 and SEQ ID NO: 15. In certain embodiments, said functionalized silk has at least 70%, such as at least 80%, such as at least 85%, preferably at least 90%, such as at least 95%, identity to an amino acid sequence selected from the group consisting of SEQ ID NO: 14 and SEQ ID NO: 15.

Thus, in one embodiment, said 2D substrate does not comprise any animal derived components. In one embodiment, said 2D substrate is not Matrigel™. In other words, the components of said 2D substrate may be components produced by using recombinant technology.

Thus, in one embodiment, said 2D substrate may comprise or consists of laminins (LN) and fragments thereof, such as recombinantly produced laminins (LN) and fragments thereof.

Laminins are high-molecular weight proteins of the extracellular matrix. They are a major component of the basal lamina which is a protein network foundation for most cells and organs. The laminins are an important and biologically active part of the basal lamina, influencing cell differentiation, migration, and adhesion. Thus, the choice of laminins for use as cell culture substrate is important in order to provide cells with the appropriate chemo- and mechanosensitive microenvironment and thus optimal culture conditions. Each laminin isoform consists of three inter-coiled chains—an α, β, and γ chain—that exist in five, four, and three genetically distinct variants, respectively. Laminin isoforms are named according to their chain composition. For example, a combination of an α5 chain, a β2 chain, and a γ1 chain, forms laminin 5-2-1 (LN-521). The trimeric proteins form a cross-like structure that can bind to other extracellular matrix molecules and various cell membrane receptors.

Thus, the choice of laminin or fragments thereof for use as 2D substrates is important and will be influenced by the cell type cultured. Furthermore, differences between cell lines, such as different ES or IPS cell lines, or primary cells may influence the choice of optimal 2D substrate. Full length laminins may be useful as cell culture substrates, however also fragments thereof may be used for cell culture. Distinct domains of laminin have been identified which mediate different activities, such as cell attachment as well as influences on cellular proliferation, differentiation and motility.

In one embodiment of the method as disclosed herein, said laminins (LN) and fragments thereof are selected from the group consisting of LN-521 and fragments thereof, LN-511 and fragments thereof, LN-332 and fragments thereof, LN-421 and fragments thereof, LN-121 and fragments thereof and LN-111 and fragments thereof; such as the group consisting of LN-521 and fragments thereof, LN-511 and fragments thereof, LN-332 and fragments thereof, LN-421 and fragments thereof and LN-121 and fragments thereof; such as the group consisting of LN-521 and fragments thereof, LN-511 and fragments thereof and LN-332 and fragments thereof; such as the group consisting of LN-521 and fragments thereof or the group consisting of LN-511 and fragments thereof.

In one embodiment, said laminins and fragments thereof are selected from the group consisting of LN-521, LN-511, LN-332, LN-421, LN-121 and LN-111; such as the group consisting of LN-521, LN-511, LN-332, LN-421 and LN-121, such as the group consisting of LN-521, LN-511 and LN-332; such as the group consisting of LN-521 and 511; such as wherein said laminins and fragments thereof are LN-521 or such as wherein said laminins and fragments thereof are LN-511.

Known are laminin E8 fragments, which are truncated proteins composed of the C-terminal regions of the α, β and γ chains. These laminin fragments contain the active integrin-binding site comprising the laminin globular 1-3 domains of the α chain and the glutamate residue in the C-terminal tail of the γ chain, but lack other activities such as the heparin/heparan sulphate-binding activity, which are associated with full lenght laminins. E8 fragments 5 represent a functionally minimal form which retains the full capability for binding to α6β1 integrin. In one embodiment, said fragment(s) thereof is/are E8 fragment(s).

Thus, in one embodiment, said laminins and fragments comprise E8 fragments of laminins, such as an E8 fragment selected from the group consisting of an E8 fragment of LN-511, an E8 fragment of LN-521, an E8 fragment of LN-332, an E8 fragment of LN-421, an E8 fragment of LN-121 and an E8 fragment of LN-111; such as the group consisting of an E8 fragment LN-511, an E8 fragment of LN-521, an E8 fragment of LN-332, and E8 fragment of 421 and an E8 fragment of LN-121; such as the group consisting of an E8 fragment LN-511, an E8 fragment of LN-521 and an E8 fragment of LN-332; such as the group consisting of an E8 fragment of LN-511 and an E8 fragment of LN-521; such as an E8 fragment of LN-511 or an E8 fragment of LN-521.

It will be appreciated that the EP cells provided in step i) of the present method may be generated by making use of various differentiation protocols, such as protocols previously described. It will be appreciated that the provision of a large number of EP cells in step i) is beneficial. Thus, it may be beneficial to generate said EP cells by making use of the methods as disclosed herein. For clarity, the usefulness of method as described in steps i)-v) herein, is by no way limited to EP cells generated by the methods disclosed herein.

In one embodiment as disclosed herein, a method is provided wherein in step i) more than approximately 30%, such as more than approximately 40%, 40%, such as more than approximately 45%, such as more that approximately 50% of the total cell population are EP cells characterized by the expression of NEUROD1. For example, the EP cells provided in step i) may be obtained in step c+1) disclosed herein. Thus, the population of EP cells in step i) may be part of a total population of cells comprising a fraction of cells which do not express markers characteristic of EP cells. In one embodiment as disclosed herein, the population of EP cells in step i) is a population comprising EP cells. In one embodiment as disclosed herein, a method is provided wherein in step i) a population of EP cells is provided, wherein more than approximately 30%, such as more than approximately 40%, such as more than approximately 45%, such as more that approximately 50% of the total cell population are EP cells, such as EP cells characterized by the expression of NKX6.1 and at least NEUROD1, such as endocrine progenitor cells characterized by the expression of NKX6.1 and at least NEUROD1. In one embodiment as disclosed herein, in step i) the population comprising EP cells, comprises more than approximately 30%, such as more than approximately 40%, such as more than approximately 45%, such as more that approximately 50% EP cells, such as EP cells characterized by the expression of NKX6.1 and at least NEUROD1, such as endocrine progenitor cells characterized by the expression of NKX6.1 and at least NEUROD1.As discussed and exemplified above other combinations of two or more of the markers PDX1, NKX6.1, NEUROD1 and NGN3 can be used as characteristic of EP cells.

In one embodiment of the method as disclosed herein, said step ii) of providing a single cell suspension of said population of EP cells is performed when more than approximately 15%, such as more than approximately 20%, such as more than approximately 25%, such as more than approximately 30%, such as more than approximately 35%, such as more than approximately 40%, such as more than approximately 45%, such as more than approximately 50% of the total cell population are EP cells characterized by the expression of NEUROD1 or NGN3. In one embodiment, said EP cells are characterized by the expression of NEUROD1 and NGN3. In one embodiment, said step ii) is performed when more than approximately 15%, such as more than approximately 20%, such as more than approximately 25%, such as more than approximately 30%, such as more than approximately 35%, such as more than approximately 40%, such as more than approximately 45%, such as more than approximately 50% of the total cell population are EP cells characterized by the expression of NKX6.1 and NEUROD1. In one embodiment, said step ii) is performed when the cells do not exhibit hormone expression, such as do not exhibit expression In one embodiment, said step ii) is performed approximately when the cells do not exhibit hormone expression, such as do not exhibit expression of insulin and/or glucagon. In this context, not exhibit expression is to be understood as do not exhibit any detectable expression, such as detectable by methods or means disclosed in the present example section. In one embodiment, said step ii) is performed before the cells exhibit hormone expression, such as do exhibit expression of insulin and/or glucagon.

In one embodiment there if provided a method for the generation pancreatic islet-like cell aggregates in vitro as defined herein, wherein step iv) of culturing of said population of EP cell in the form of 3D structures in 3D culture conditions permissive of differentiation pancreatic monohormonal β-cells to provide pancreatic islet-like cell aggregates, comprises is culture in a culture medium suitable for culture of endocrine progenitor cells under conditions permissive of differentiation into monohormonal β-cells. Non limiting examples of stage 6 (S6) medium are as defined in the present Examples. The skilled person appreciates that other suitable media may be used. Said culture medium in step iv) may be supplemented by other factors a specified herein.

Trolox (6-hydroxy-2,5,7,8-tetramethylchroman-2-carboxylic acid) is a water-soluble analog of vitamin E with a powerful antioxidant effect. Trolox is also a powerful inhibitor of membrane damage. In one embodiment said medium in step iv) comprises approximately from 5 to 15 μM of Trolox, such as approximately 10 UM of Trolox. As the skilled person appreciates, it is possible to replace Trolox with a derivate or agonist thereof.

N-Acetyl-L-cysteine is cell culture component for example for intestinal basal medium for the culture of mouse intestinal stem cells and also as a component of expansion medium. In one embodiment said medium in step iv) comprises approximately from 0.5 to 3 mM of N-Acetyl-L-cysteine, such as approximately 1 mM of N-Acetyl-L-cysteine. As the skilled person appreciates, it is possible to replace N-Acetyl-L-cysteine with a derivate or agonist thereof.

H1152 is a Rho kinase inhibitor and is a cell-permeable, highly specific, reversible, potent, and ATP-competitive inhibitor of Rho-associated kinase (ROCK). As the skilled person appreciates, it is possible to replace H1152 with a derivate or agonist thereof.

GC-1 is a thyroid hormone receptor (TR) agonist and is more potent than the thyroid hormone T3. Thyroid hormone T3 is important for the development of β-cells and is discussed in more detail below. In one embodiment said medium in step iv) comprises approximately from 0.5 M to 3 M of GC-1, such as approximately 1 μM of GC-1. As the skilled person appreciates, it is possible to replace GC-1 with a derivate or agonist thereof.

In one embodiment, step iv) of the method as disclosed herein comprises culturing the EP cell population in a culture medium which comprises H1152, GC-1, a Trolox and N-acetyl-L-cysteine. In one embodiment, step iv) of the method as disclosed herein comprises culturing the EP cell population in a culture medium which comprises H1152, GC-1, Trolox and N-acetyl-L-cysteine for approximately 3 weeks followed by culture in media comprising approximately GC-1, Trolox, N-acetyl-L-cysteine but not H1152.

In one embodiment, step iv) of the method as disclosed herein comprises culturing the EP cell population in a culture medium which comprises approximately 10 UM H1152, approximately 1 μM GC-1, approximately 10 μM Trolox and approximately 1 mM N-acetyl-L-cysteine. In one embodiment, step iv) of the method as disclosed herein comprises culturing the EP cell population in a culture medium which comprises approximately 10 μM H1152, approximately 1 μM GC-1, approximately 10 μM Trolox and approximately 1 mM N-acetyl-L-cysteine for approximately 3 weeks followed by culture in media comprising approximately 1 μM GC-1, approximately 10 μM Trolox and approximately 1 mM N-acetyl-L-cysteine but not H1152.

In one embodiment, said culture in step iv) is on a shaker, such as an orbital shaker.

As discussed in the context of the present method, the inventors have found that the timing of transferring the EP cells from culture on a 2D substrate to culture on a 3D substrate is important for obtaining enriched pancreatic islet-like cell aggregates in vitro, which are enriched for β-cells and exhibit low percentage of other monohormonal cells, low percentage of polyhormonal cells and low percentage of proliferating cells.

In one embodiment of said method, the cells are not transferred from culture on a 2D substrate to culture on a 3D substrate prior to exhibiting expression of markers characteristic of endocrine progenitor cells. Endocrine progenitor cells are characterized by the expression of PDX1, NKX6.1 and at least one of NEUROD1 and NGN3, as discussed above. As explained above, EP cells do not express pancreatic hormones. In one embodiment, said step ii) is performed approximately 24 hours after the initiation of expression of NEUROD1 and/or NGN3 by said EP cells, such as 24 hours after initiation of expression of NEUROD1 and/or NGN3 by said EP cells in more than 15%, such as more than approximately 20%, such as more than approximately 25%, such as more than approximately 30%, such as more than approximately 35%, such as more than approximately 40%, such as more than approximately 45%, such as more than approximately 50% of the total cells in the culture. In one embodiment, said step ii) is performed within 6, such as within 5 such as within 1-4, such as 1-3, such as 1-2 or 2-3 days after the initiation of expression of NEUROD1 or NGN3 by said EP cells. Step ii) is I performed prior to expression of INSULIN and/or GLUCAGON.

As discussed above, in one embodiment, the formation of 3D structures is step iii) is spontaneous formation of 3D structures. For example, such spontaneous formation of 3D structures may occur via self-aggregation. Alternatively, said formation of 3D structures is step iii) is forced (also referred to as aided) formation of 3D structures. Non-limiting examples of forced (or aided) formation of 3D structures include culture conditions wherein the cells are forced close together via the shape of the cell culture vial or flask. In one embodiment, the 3D culture conditions allow for self-aggregation of the cells. Without being bound by theory, the present Examples show that self-aggregation allows for selective enrichment of endocrine progenitor cells, which develop to stage 6 cells. Additionally, said selective enrichment results in increased generation of pancreatic monohormonal β-cells in said cultures. In one embodiment, said pancreatic monohormonal β-cells are generated as part of cell aggregates. In one embodiment, said aggregates comprise monohormonal β-cells. In one embodiment, said pancreatic monohormonal β-cells are generated as part of pancreatic islet-like cell aggregates in vitro. In one embodiment said pancreatic islet-like cell aggregates in vitro further comprise pancreatic monohormonal cell alpha and/or delta cells.

In one embodiment, said pancreatic islet-like cell aggregates in vitro are generated from human ES cells and comprise at least 40%, such as at least 45%, such as at least 50% such as at least 55% β-cells, such as at least 60% β-cells, such as at least 65% monohormonal β-cells. In one embodiment, said pancreatic islet-like cell aggregates in vitro are generated from human iPS cells and comprise at least 40% β-cells, such as at least 45%, such as at least 50% such as at least 55% β-cells, such as at least 60% β-cells, such as at least 65% monohormonal β-cells. In one particular embodiment, said IPS cells are C₇ cells and said islet-like cell aggregates comprise at least 60% β-cells, such as at least 65% monohormonal β-cells. In one embodiment, said monohormonal β-cells are characterized by INSULIN expression.

Thus the present inventors have demonstrated that the present invention is equally applicable to differentiation for ES and iPS cells and that the advantageous effects thereof is not limited to the specific cell lines.

In one embodiment, said pancreatic islet-like cell aggregates in vitro comprise at most approximately 5%, such as at most approximately 4%, such as at most approximately 3%, such as at most approximately 2%, such as at most approximately 1% proliferating cells, such as proliferating cells expressing Ki-67. In one embodiment, said pancreatic islet-like cell aggregates in vitro comprise at most approximately 5%, such as at most approximately 4%, such as at most approximately 3%, such as at most approximately 2%, such as at most approximately 1% proliferating cells, such as proliferating cells expressing Ki-67. In one embodiment, said pancreatic islet-like cell aggregates in vitro comprise at most approximately 5%, such as at most approximately 4%, such as at most approximately 3%, such as at most approximately 2%, such as at most approximately 1% proliferating cells, such as proliferating cells expressing Ki-67. In one embodiment, said Ki-67 is scored at one day after aggregate formation. In one embodiment, said Ki-67 is scored on day 15 of culture. Thus, said evaluation may be after a duration of step iv) of approximately 4 weeks.

In one embodiment, said pancreatic islet-like cell aggregates in vitro comprise at least 50%, such as at least 55%, such as at least 60%, such as at least 65%, such as at least 70% such as at least 75% cells expressing NEUROD1 and NKX6.1. In one embodiment, said pancreatic islet-like cell aggregates in vitro comprise approximately from 60 to 90%, such as approximately from 60 to 80%, such as approximately from 65 to 80% cells expressing NEUROD1 and NKX6.1. In one embodiment, said pancreatic islet-like cell aggregates in vitro comprise at least 50%, such as at least 80%, such as at least 85%, such as at least 87%, such as at least 90% cells expressing NEUROD1. In one particular embodiment, said pancreatic islet-like cell aggregates in vitro are generated from human ES cells. In one embodiment, said expression of NEUROD1 and/or NKX6.1 is scored on day 15 of culture. Thus, said evaluation may be at step iv) after the formation of said aggregates. In one embodiment, step iv) comprises culturing said population of cells for approximately 2 weeks or longer, such as for approximately 3 weeks or longer, such as for approximately from 3 to 5 weeks, such as for approximately 4 weeks.

As used herein, the term “monohormonal” refers to cells that express only one type of hormone. For example, to monohormonal β-cells that expression only insulin and do not express other hormones that are expressed by pancreatic islet cells, such as glucagon or somatostatin. As used herein, the term “polyhormonal” refers to cells which express at least two different hormones.

β-cells in vivo are monohormonal cells and it is beneficial that the population obtained by the inventive method exhibits the properties of natural β-cells in vivo, in other words endogenous β-cells in vivo, such as properties of healthy natural β-cells in vivo, or healthy endogenous β-cells in vivo.

Thus, in one embodiment of the present method as disclosed herein, said islet-like cell aggregates comprising β-cells generated in step v) comprise monohormonal β-cells. In one embodiment of the present method, said monohormonal β-cells in step v) express of insulin. In one embodiment, said monohormonal β-cells in step v) express C-peptide upon glucose stimulation. In particular, said monohormonal β-cells in step v) do not express glucagon or somatostatin. In particular, said monohormonal β-cells in step v) do not express glucagon and somatostatin.

In one embodiment, said monohormonal β-cells in step v) are characterized by the expression of insulin. Said monohormonal β-cells may further express at least one of NKX6.1, PDX1 and NEUROD1.

In one embodiment, said monohormonal β-cells are characterized by the expression of insulin and PDX1. In one embodiment, said monohormonal β-cells are characterized by the expression of insulin and NKX6.1. In one embodiment, said monohormonal β-cells are characterized by the expression of insulin and NEUROD1. In one embodiment, said monohormonal β-cells are characterized by the expression of insulin and two of NKX6.1, PDX1 and NEUROD1; such as insulin, NKX6.1 and PDX1; or insulin, NKX6.1 and NEUROD1; or insulin, PDX1 and NEUROD1. In one embodiment, said monohormonal β-cells are characterized by the expression of insulin, PDX1, NKX. 1 and NEUROD1.

In one embodiment, there is provided a method for the generation of pancreatic islet-like cell aggregates in vitro as defined herein, wherein said islet-like cell aggregates in v) comprise at least least approximately 25%, such as at least approximately 30%, such as at least approximately 35%, such as at least approximately 40%, such as at least approximately 45%, such as at least approximately 50% such as at least approximately 55% β-cells, such as at least approximately 60% monohormonal β-cells, such as at least approximately 65% β-cells. In one embodiment said pancreatic islet-like cell aggregates in vitro in v) comprise approximately from 25 to 70%, such as approximately from 30 to 70%, such as approximately from 40 to 70%, such as approximately from 40 to 60% monohormonal β-cells.

In one embodiment, said pancreatic islet-like cell aggregates in vitro in v) comprise at least 40%, such as at least 45%, such as at least 50% such as at least 55% β-cells, such as at least 60% β-cells, such as at least 65%, such as at least 70% β-cells monohormonal β-cells. In one embodiment, said monohormonal β-cells are characterized by INSULIN expression.

In one embodiment, said pancreatic islet-like cell aggregates in vitro in v) comprise at most approximately 20%, such as at most approximately 18%, such as at most approximately 16%, such as at most approximately 13%, such as at most approximately 10% monohormonal alpha cells. In one embodiment, said monohormonal alpha cells are characterized by GLUCAGON expression.

In one embodiment, there is provided a method for the generation pancreatic islet-like cell aggregates in vitro as defined herein, wherein said islet-like cell aggregates in v) comprise monohormonal β-cells and alpha cells comprise at most 5% of cells any one or more of cells selected form the group consisting of delta cells, acinar cells, ductal cells and activated stellate cells. In one embodiment of said method, said islet-like cell aggregates in v) comprise at most approximately 5%, such as at most approximately 4, 3, 2 or 1% polyhormonal cells. In one embodiment of said method, said islet-like cell aggregates in v) comprise at most approximately 5%, such as at most approximately 4, 3, 2 or 1% non-endocrine cells.

In one embodiment, said pancreatic islet-like cell aggregates in vitro in v) are scored at the end of S6, such on day 38-42 of culture, such as at day 38, 39, 40, 41, 42 or later.

In addition, the present inventor have shown that a short culture time of posterior foregut (PF) cells in conditions which are permissive of differentiation into pancreatic progenitor (PP) cell gives rise to a larger number of endocrine progenitor (EP) cells (also referred to herein as endocrine precursor cells), even though the number of obtained PP cells is lower than in corresponding methods with longer culture time. As used herein, the terms “endocrine progenitor (EP) cells” and “endocrine precursor (EP) cells” are used interchangeably. Without being bound by theory, it appears that PP cells obtained by the present method comprising said short culture time are competent to develop/differentiate into EP cells. Importantly, the EP cells obtained by the method as disclosed herein have the potential to develop into mature and functional pancreatic β-cells, such as to monohormonal β-cells which have the ability to respond to glucose stimulation by expression of C-peptide. As used herein, the term “corresponding method” refers to a method in which all steps are the same, except if the step indicated specifically. Thus, the corresponding method is to be interpreted as the same method but which differs in the step indicated, for example the step may relate to the time of culture, such as time of culture during step b-1); or b); or both b-1) and b).

Thus, in one embodiment there is provided a method for the generation pancreatic islet-like cell aggregates in vitro wherein prior to step i) the method comprises the steps a)-c) of
a) providing a cell population of posterior foregut cells, such as posterior foregut cells characterized by expression of PDX1;
b) culturing said cell population of posterior foregut cells for no more than approximately 78 hours, such as no more than approximately 72 hours, under conditions permissive of differentiation into pancreatic progenitor cells; and
c) thereby generating a cell population of pancreatic progenitor cells, such as pancreatic progenitor cells characterized by expression of both PDX1 and NKX6.1.

To clarify, the cells of the cell population of posterior foregut (PF) cells characterized by expression of PDX1 provided in step a) do not express NKX6.1, in other words lack expression of NKX6.1.

Thus, in one embodiment of the method as disclosed herein, the cell population of posterior foregut (PF) cells provided in step a) is characterized by expression of PDX1 and lack of expression of NKX6.1. In one embodiment, the cell population of PF cells provided in step a) is further characterized by the expression of HNF6. Thus, it will be understood that the posterior foregut cells may be characterized by the expression HNF6, PDX1 or by co-expression of PDX1 and HNF6.

In one embodiment of the first aspect as disclosed herein, there is provided a method, wherein in step b) said cell population is cultured for no more than approximately 75 hours, such as no more than approximately 72 hours, such as no more than approximately 66 hours, such as no more than approximately 60 hours, such as no more than approximately 48 hours. In another embodiment, there is provided a method wherein in step b) said cell population is cultured for a time period of approximately from 42 to 78 hours, such as a period of approximately from 44 to 76 hours, such as a period of approximately from 46 to 74 hours, such as a period of approximately from 48 to 72 hours. In one embodiment, said cell population is cultured for a time period of approximately from 40 to 78 hours, such as a period of approximately from 42 to 76 hours, such as a period of approximately from 44 to 74 hours, such as a period of approximately from 48 to 72 hours. In one embodiment, said cell population is cultured for a time period of approximately from 42 to 54 hours, such as a period of approximately from 44 to 52 hours, such as a period of approximately from 46 to 50 hours, such as a period of approximately 48 hours.

In one embodiment of the method as disclosed herein, the cell population of pancreatic progenitor cells, such as pancreatic progenitor cells characterized by the expression of PDX1 and NKX6.1, in step c) is further characterized by expression of at least one marker selected from the group consisting of PTF1A, SOX9, HNF6 and CPA, such as a marker selected from the group consisting of SOX9 and PTF1A. In one embodiment of the method as disclosed herein, the cell population of pancreatic progenitor cells in step c) is further characterized by expression PTF1A and SOX9.

As discussed above, the term “conditions permissive of differentiation” refers to conditions which allow cells to develop (in other words to differentiate) such that they exhibit the characteristics of said cell type and may include combinations of cell culture media, presence and/or absence of extrinsic factors as well as timing thereof. In one embodiment, said culture medium in step b) is a culture medium suitable for culture of posterior foregut cells under conditions permissive of differentiation into pancreatic progenitor cells. Non limiting examples of stage 4 (S4) medium are as defined in the appended Examples. Said culture medium in step b) may be supplemented by other factors as specified herein, The skilled person appreciates that other suitable media may be used. In particular, culturing under conditions permissive of differentiation into pancreatic progenitor cells in step b) as disclosed herein may be related to culturing the cell population in a culture medium in the presence of specific extrinsic factors, for example epidermal growth factor (EGF) and nicotinamide (NIC) or derivatives or agonists thereof. Thus, in one embodiment of the method as disclosed herein, conditions permissive of differentiation into pancreatic progenitor cells comprises culturing said cell population in a culture medium in the presence of an effective amount of epidermal growth factor (EGF), such as human EGF, or a derivative or an agonist thereof; and an effective amount of nicotinamide (NIC) or a derivative or an agonist thereof. In one embodiment of the method as disclosed herein, step b) comprises culturing said cell population in a culture medium in the presence of an effective amount of EGF, such as human EGF, and an effective amount of NIC.

Epidermal growth factor (EGF) is a protein that stimulates cell growth and differentiation by binding to its receptor, EGFR. Human EGF is 6-kDa protein and has 53 amino acid residues and three intramolecular disulfide bonds. Binding to the receptor stimulates ligand-induced dimerization, activating the intrinsic protein-tyrosine kinase activity which initiates a signal transduction cascade that results in a variety of biochemical changes within the cell—a rise in intracellular calcium levels, increased glycolysis and protein synthesis, and increases in the expression of certain genes including the gene for EGFR —that ultimately lead to DNA synthesis and cell proliferation. EGF is a member of the EGF-family of proteins. Members of this protein family have highly similar structural and functional characteristics. Besides EGF itself other family members include Heparin-binding EGF-like growth factor (HB-EGF), transforming growth factor-α (TGF-α), Amphiregulin (AR), Epiregulin (EPR), Epigen, Betacellulin (BTC), neuregulin-1 (NRG1), neuregulin-2 (NRG2), neuregulin-3 (NRG3) and neuregulin-4 (NRG4).

The skilled person will appreciate that the term “epidermal growth factor (EGF) or a derivative or agonist thereof” as used herein is meant to include factors that potentiate or substitute for EGF signaling. Such factors may be involved in signaling downstream of EGF or be small molecule agonists. A non-limiting list of EGF derivatives or agonists include high-affinity EGFR ligands such as TGF-α, BTC and HB-EGF as well as and low-affinity ligands such as AR, EPR and Epigen. Thus, in one embodiment of the present aspect, said EGF or derivative or agonist thereof is selected from a group consisting of EGF, TGF-α, BTC, HB-EGF, AR, EPR and Epigen, such as is EGF. In one particular embodiment, said EGF is human EGF.

Nicotinamide (NIC), also known as NAM, is a form of vitamin B. The structure of nicotinamide consists of a pyridine ring to which a primary amide group is attached in the meta position and it is an amide of nicotinic acid. Nicotinamide is well known as a cell culture supplement used in the differentiation of embryonic stem and induced pluripotent stem cells and has been shown to modulate stem cell differentiation in various applications, including differentiation of pancreatic cells. The skilled person will appreciate that the term “nicotinamide (NIC) or a derivative or agonist thereof” as used herein is meant to include factors that potentiate or substitute for NIC signaling. Non-limiting examples of such derivatives or agonists include NIC, niacin (nicotinic acid), nicotinamide riboside, NAD/NADP as well as tryptophan, which is a precursor of NIC. Thus, in one embodiment of the present aspect, said NIC or derivative or agonist thereof is selected from a group consisting of NIC, niacin, nicotinamide riboside, NAD/NADP and tryptophan, such as wherein said NIC or derivative or agonist thereof is NIC.

In one embodiment of said method in step b), said effective amount of EGF or a derivative or agonist thereof is approximately from 50 to 200 ng/mL, such as approximately from 50 to 150 ng/ml, such as approximately from 75 to 125 ng/ml, such as approximately 100 ng/ml

In one embodiment of said method in step b), said effective amount of NIC or a derivative or agonist thereof is approximately 5 nM-20 nM, such as approximately from 5 to 15 mM, such as approximately from 8 to 12 mM, such as approximately 10 mM.

Step b) of the method as disclosed herein may comprise culturing the cell population in a culture medium which comprises additional factors, such as one or more factors selected from KGF and derivates and agonists thereof; ActA and derivates and agonists thereof; retinoic acid and derivates and agonists thereof; SANT-1 and derivates and agonists thereof; PDBu and derivates and agonists thereof; and LDN and derivates and agonists thereof, such as one of more factors selected from KGF, ActA, retinoic acid, SANT-1, PDBu and LDN. In one embodiment, said step b) comprises culturing said cell population in a culture medium further comprising KGF or derivates or agonists thereof; ActA or derivates or agonists thereof; retinoic acid or derivates or agonists thereof; SANT-1 or derivates or agonists thereof; PDBu or derivates or agonists thereof; and LDN or derivates or agonists thereof, such as culture medium further comprising KGF, ActA, retinoic acid, SANT-1, PDBu and LDN. It will be appreciated that the culture medium may comprise a mixture of a factor and its derivative and/or agonist.

The skilled person will appreciate that the cell culture medium and factors comprised therein may be adapted by the replacement of factors by their derivatives and/or agonist, for example by the ones described below.

Keratinocyte Growth Factor (KGF), also known as Fibroblast Growth Factor 7 (FGF7,) is a member of the FGF-family of proteins. It is bioactive protein intended for often used in cell culture applications. KGF binds to fibroblast growth factor receptor 2b (FGFR2b). KGF induces proliferation for many epithelial cells but not for fibroblasts and endothelial cells, it is a major growth factor for skin keratinocytes and is also used in culture and differentiation of pluripotent cells. The skilled person will appreciate that KGF may be replaced in the cell culture described herein by a derivative or an agonist thereof. Non-limiting examples of such agonists include other factors that bind to FGFR2b and signal through said receptor, such as FGF10.

In one embodiment, said KGF or derivate or agonist thereof is selected from the group consisting of KGF and FGF10, such as is FGF10.

In one embodiment, said culture medium in step b) comprises 25 to 75 ng/ml KGF or derivate or agonist thereof, such as 50 ng/mL KGF or derivate or agonist thereof. In one embodiment said medium in step b) comprises approximately from 25 to 75 ng/ml of KGF, such as approximately 50 ng/mL of KGF.

ActA, or Activin A, is a member of the TGF-beta superfamily. Activin, as well as Nodal ligands, can both signal through the same receptors and effectors in order to regulate transcription. In many cases, the effects of Nodal and Activin-mediated signalling are indistinguishable; hence, they are referred to as the Activin/Nodal pathway. Activin/Nodal bind to type II Activin receptors (ActRII/IIB), leading to the recruitment, phosphorylation and activation of type

I Activin receptors (Activin receptor-like kinases, or ALKs, including ALK1-7), in particular of ALK4. The serine/threonine kinase receptors ActRII/IIB and ALK4/7 then trigger the phosphorylation of the Smad transcription factors Smad2 and Smad3.

It is known in the art that TGFβ signaling is involved in embryogenesis, cell differentiation and apoptosis as well as in other functions. The Activin/Nodal and TGFβ pathways share the downstream effectors Smad2 and Smad3. Activin/Nodal have been reported to be involved in maintaining pluripotency of stem cells, however Activin/Nodal signalling is also required for endoderm differentiation.

The skilled person will appreciate that ActA may be replaced in the cell culture described herein by a derivative or an agonist thereof. Examples of such agonists include Nodal which signal through said receptor, downstream effect molecules, such as Smad 2 and 3 as well as TGFβ which signals through the same effector molecules. Additional derivative or an agonist thereof include, but are not limited to TGFbeta1-3 (TGFβ1, TGFβ2, TGFβ3) Nodal, Activin A, GDF-1, GDF-8, GDF-11 that all activate Smad2/3/4 complex.

In one embodiment, said ActA or derivate or agonist thereof is selected from the group consisting of ActA and GDF-8, Nodal, TGFbeta1-3 (TGFβ1, TGFβ2, TGFβ3). In one embodiment said medium in step b) comprises approximately from 1 to 10 ng/mL, such as from 2.5 to 7.5 ng/mL of ActA, such as approximately 5 ng/mL of ActA.

Retinoic acid (RA) is a metabolite of vitamin A and mediates the functions of vitamin A required for growth and development. Retinoic acid is known to be involved in specifying the position along the embryonic anterior-posterior axis, also referred to as patterning and is also known to play a role in the later stage of pancreas development to promote the generation of pancreatic endocrine progenitors and their differentiation into islets and β-cells. Retinoic acid acts by binding to the retinoic acid receptor (RAR), which is bound to DNA as a heterodimer with the retinoid X receptor (RXR) in regions called retinoic acid response elements (RAREs). Binding of the retinoic acid ligand to RAR alters the conformation of the RAR, which affects the binding of other proteins that either induce or repress transcription of nearby genes. The skilled person will appreciate that the term “retinoic acid or a derivative or agonist thereof” as used herein is meant to include factors that potentiate or substitute for retinoic acid signaling. Such factors may be involved in signaling downstream of retinoic acid or be small molecule agonists. A non-limiting list of retinoic acid derivatives or agonists include all-trans retinoic acid, synthetic retinoid ec23, Ch55, TTNPB, fenretinide, RAR agonists, such as RARA agonists and RARB agonists AC261066, adapalene, AC55649, AM80, AM580, BMS 753, tazarotene and Ro 41-5253. Thus, in one embodiment of the present aspect, said retinoic acid or derivative or agonist thereof is selected from a group consisting of retinoic acid, all-trans retinoic acid, synthetic retinoid ec23, Ch55, TTNPB, fenretinide, RAR agonists, such as RARA agonist and RARB agonist AC261066, adapalene, AC55649, AM80, AM580, BMS 753, tazarotene, Ro 41-5253.

In one embodiment, said retinoic acid (RA) or derivative or agonist thereof is selected from a group consisting of retinoic acid, all-trans retinoic acid and synthetic retinoid ec23. In one embodiment, said retinoic acid or derivative or agonist thereof is selected from a group consisting of retinoic acid and all-trans retinoic acid. In one embodiment, said retinoic acid or derivative or agonist thereof is all-trans retinoic acid. In one embodiment said medium in step b) comprises approximately from 50 to 150 nM of RA or derivative or agonist thereof, such as approximately 100 nM of RA or derivative or agonist thereof.

In one embodiment said medium in step b) comprises approximately from 50 to 150 nM of RA, such as approximately 100 nM of RA.

Hedgehog (HH or Hh) signaling is known to play a key role in regulating vertebrate organogenesis, such as in the growth of digits on limbs and organization of the brain. The vertebrate hedgehog protein family consists of sonic hedgehog (SHH), indian hedgehog (IHH) and desert hedgehog (DHH), which signal through a similar pathway and share many functional characteristics. A critical negative regulator of pancreatic development is sonic hedgehog (SHH) and thus it is of importance to repress SHH at the initiation of pancreatic development and hedgehog suppression must be maintained to ensure proper pancreatic development.

Briefly, Hh signals by interacting with the Hh receptor complex comprising two components; Patched (Ptc) and Smoothened (Smo) that transduce the Hh signal into the cell. Ptc is considered to repress Hh signaling by binding to Smo in the cell membrane. In the presence of Hh ligand, this repression is relieved and Smo is able to signal. In vertebrates, the zinc finger proteins Gli1, Gli2 and Gli3 are downstream mediators of Hh signaling and are involved in controlling the transcriptional response of target genes in a Hh dependent manner.

SANT-1 is an inhibitor of hedgehog (Hh) signaling and acts by antagonizing smoothened activity. Non-limiting examples of Inhibitors of hedgehog signaling include cyclopamine, IHR 1, IHR-Cy3, Itraconazole, Jervine, M 25, MRT 10, PF 04449913 maleate, PF 5274857 hydrochloride, SANT-1 and SANT-2.

In one embodiment, said SANT-1 or derivate or agonist thereof is selected from the group consisting of cyclopamine, IHR 1, IHR-Cy3, Itraconazole, Jervine, M 25, MRT 10, PF 04449913 maleate, PF 5274857 hydrochloride, SANT-1 and SANT-2.

In one embodiment said medium in step b) comprises approximately from 0.10 to 0,0.50 μM of SANT-1, such as approximately 0.25 μM of SANT-1.

PDBu (Phorbol-12,13-dibutyrate) is a strong promoter of nitric oxide (NO) synthesis and a potent activator of protein kinase C. PDBu has been reported to be a tumor promoter that activates a variety of cellular responses, including proliferation. It is possible to replace PBDu with other activators of protein kinase C, for example but not limited to phorbol 12-myristate 13-acetate (PMA) or TPPB. In one embodiment, said PDBu or derivate or agonist thereof is selected from the group consisting of PDBu, PMA and TPPB. In one embodiment said medium in step b) comprises approximately from 0.25 to 0.75 μM of PDBu, such as approximately 0.5 μM of PDBu.

LDN193189 (referred to herein as LDN) is an inhibitor of the bone morphogenetic (BMP) pathway and acts by inhibiting ALK2 and ALK3. LDN functions primarily through prevention of Smad1, Smad5, and Smad8 phosphorylation. LDN is analog of dorsomorphin and noggin and dorsomorphin may replace LDN. In one embodiment, said LDN or derivate or agonist thereof is selected from the group consisting of LDN, Noggin and dorsomorphin. In one embodiment said medium in step b) comprises approximately from 100 to 300 nM of LDN, such as from 100-250 nM or LDN, such as 150-250 nM LDN, such as approximately 200 nM of LDN.

In one embodiment, step b) of the method as disclosed herein comprises culturing the cell population in a culture medium which comprises approximately 50 ng/ml KGF, approximately 5 ng/mL ActA, approximately 100 nM retinoic acid, approximately 0.25 mM SANT-1, approximately 500 nM PDBu, approximately 200 nM LDN, approximately 100 ng/mL EGF and approximately 10 mM NIC.

As explained above, the present disclosure also describes that a short culture time of posterior foregut (PF) cells under conditions which are permissive of differentiation into pancreatic progenitor (PP) cell gives rise to a larger number of endocrine progenitor (EP) cells compared to corresponding method with longer culture time, even though the number of obtained PP cells is lower than in corresponding methods with longer culture time.

Thus, in one embodiment of the present aspect, there is provided a method wherein at least approximately 70%, such as at least approximately 75%, such as at least approximately 80% of the posterior foregut cells in a) differentiate into pancreatic progenitor cells in c).

In one embodiment, there is provided a method as disclosed herein, wherein in step c) at least approximately 80%, such as approximately from 80 to 85%, such as approximately from 80 to 90%, of the total cell population express PDX1. In one embodiment, there is provided a method as disclosed herein, wherein in step c) at most approximately 10%, such as at most approximately 8%, such as at most approximately 7%, such as at most approximately 5%, such as at most approximately 3% of the total cell population express NEUROD1. In one embodiment, there is provided a method as disclosed herein, wherein in step c) at most approximately 10%, such as at most approximately 8%, such as at most approximately 7%, such as at most approximately 5%, such as at most approximately 3% of the total cell population express NEUROD1 and do not express NKX6.1. In one embodiment, there is provided a method as disclosed herein, wherein in step c) approximately from 25 to 50%, such as approximately from 25 to 48%, such as approximately from 25 to 45% of the total cell population express NKX6.1. In one embodiment, there is provided a method as disclosed herein, wherein in step c) approximately from 30 to 50%, such as approximately from 35 to 45%, such as approximately from 40 to 45% of the total cell population express NKX6.1. In one embodiment, in step c) approximately at least approximately 40%, such as at least approximately 50%, such as at least approximately 60% of the cells do not express NEUROD1 and NKX6.1. In one embodiment, said posterior foregut cells are characterized by expression of PDX1 and lack expression of NKX6.1 in a).

In one embodiment of the method for the generation pancreatic islet-like cell aggregates in vitro as disclosed herein, prior to step i) the method comprises the steps a−1)-c−1) of:
a−1) providing a cell population of primitive gut tube cells, such as primitive gut tube cells characterized by expression of HNF1β and/or HNF4α;
b-1) culturing said cell population of primitive gut tube cells for no more than approximately 54 hours under conditions permissive of differentiation into posterior foregut cells; and
c−1) thereby generating a population of posterior foregut cells, such as posterior foregut cells characterized by the expression of PDX1. It will be understood that the posterior foregut cells may equally well be characterized by the expression HNF6 or by co-expression of PDX1 and HNF6.

In one embodiment of said method, said steps a−1)-c−1) are performed prior to said steps a)-c).

In one embodiment of the method for the generation pancreatic islet-like cell aggregates in vitro as disclosed herein, prior to step i) the method comprises the steps a−1)-c−1) and the steps a) and c) of
a−1) providing a cell population of primitive gut tube cells, such as primitive gut tube cells characterized by expression of HNF1β and/or HNF4α;
b-1) culturing said cell population of primitive gut tube cells for no more than approximately 54 hours under conditions permissive of differentiation into posterior foregut cells;
c−1) thereby generating a population of posterior foregut cells, such as posterior foregut cells characterized by the expression of PDX1.
a) providing the cell population of posterior foregut cells generated in step c−1), such as posterior foregut cells characterized by expression of PDX1;
b) culturing said cell population of posterior foregut cells for no more than approximately 78 hours, such as no more than approximately 72 hours, under conditions permissive of differentiation into pancreatic progenitor cells; and
c) thereby generating a cell population of pancreatic progenitor cells, such as pancreatic progenitor cells characterized by expression of both PDX1 and NKX6.1.

As used herein, each method step is designated by a letter code, such as a), b), c) etc. A full method group of culturing a cell population of a certain developmental stage under appropriate conditions for allowing it to differentiate to a population of the next developmental stage comprises the steps a), b), c) etc. Each step may further be designated by the letter code with associated numeric indicator. The numeric indicator indicates if each step in a full method group proceeds (negative numeric indicator) or follows (positive numeric indicator) the full method group of culturing posterior foregut (PF) cells to generate pancreatic progenitor (PP) cells. To illustrate the method steps in relation to the full method group of culturing a cell population of a certain developmental stage under appropriate conditions for allowing it to differentiate to a population of the next developmental stage are represented in the Table 1 below:

TABLE 1
Summary of method groups and steps. Herein steps
a + 2) to c + 2) are also referred to as steps i)-v)
(in other words steps i), ii), iii), iv) and v)).

| | | Numeric |
|---|---|---|
| Method group | Method steps | indicator |
| Primitive gut tube | a − 1 | −1 |
| cells to posterior | b − 1 | −1 |
| foregut cells | c − 1 | −1 |
| Posterior foregut | a | N/A |
| cells to pancreatic | b | N/A |
| progenitor cells | c | N/A |
| Pancreatic progenitor | a + 1 | +1 |
| cells to endocrine | b + 1 | +1 |
| progenitor cells | c + 1 | +1 |
| Endocrine progenitor | a + 2 | +2 |
| cells to mature | (corresponding to step i-iii) | |
| pancreatic β-cells | b + 2 | +2 |
| | (corresponding to step iv) | |
| | c + 2 | +2 |
| | (corresponding to step v) | |

In one embodiment of the method as disclosed herein, the cell population of primitive gut tube cells provided in step a−1) is characterized by expression of HNF1β, HNF4a or both HNF1β and HNF4α.

In one embodiment, said population of posterior foregut cells is characterized by the expression of PDX1 or HNF6. In one embodiment, said population of posterior foregut cells is characterized by the expression of PDX1 and HNF6. In one embodiment of the method as disclosed herein, the cell population of posterior foregut cells in step c−1) is characterized by expression of PDX1 and lack of expression of NKX6.1.

In one embodiment, said posterior foregut cell population in c−1) is characterized by expression of PDX1 and HNF6 and lack expression of NKX6.1. Said population of posterior foregut cells does not express NKX6.1, PTF1A and SOX9.

Without being bound by theory, it is envisioned it is beneficial that the step b-1) of culturing said cell population of primitive gut tube cells under conditions permissive of differentiation into posterior foregut cells does not exceed 54 hours in order to obtain a population of posterior foregut cells which are competent for development into later stages of the β-cell lineage. It is envisioned that primitive gut tube cells cultured under conditions permissive of differentiation into posterior foregut cells for longer periods of time than 54 hours are less competent for development into later stages of the β-cell lineage. In one embodiment of the method as disclosed herein, the cell population in step b-1) is cultured for no more than approximately 52 hours, such as no more than approximately 50 hours, such as no more than approximately 48 hours, such as no more than approximately 44 hours, such as no more than approximately 40 hours, such as no more than approximately 36 hours, such as no more than approximately 32 hours, such as no more than approximately 28 hours, such as no more than approximately 26 hours, such as approximately 24 hours.

In one embodiment of the method as disclosed herein, the cell population in step b-1) is cultured for a time period of approximately from 18 to 54 hours, such as a period of approximately from 20 to 52 hours, such as a period of approximately from 22 to 50 hours, such as a period of approximately from 24 to 48 hours. In one embodiment the cell population step b-1) is cultured for a time period of approximately from 42 to 54 hours, such as a period of approximately from 44 to 52 hours, such as a period of approximately from 46 to 50 hours, such as approximately 48 hours. In one embodiment the cell population step b-1) is cultured for a time period of approximately from 18 to hours, such as a period of approximately from 20 to 28 hours, such as a period of approximately from 22 to 26 hours, such as approximately 24 hours.

As discussed above, “conditions permissive of differentiation” refers to conditions which allow cells to develop/differentiate to exhibit the characteristics of said cell type and may include combinations of cell culture media, presence and/or absence of extrinsic factors as well as timing thereof. Said factors, as well as their derivatives and agonists have been discussed in relation to step b) above in detail and said discussion will not be repeated here for the sake of brevity only.

In one embodiment, said culture medium in step b-1) is a culture medium suitable for culture of primitive gut tube cells under conditions permissive of differentiation into posterior foregut cells. Non limiting examples of stage 3 (S3) medium are as defined in the present Examples. The skilled person appreciates that other suitable media may be used. Said culture medium in step b-1) may be supplemented by other factors a specified herein. Thus, in one embodiment there is provided a method wherein step b-1) comprises culturing said cell population in a culture medium comprising one of more factors selected from KGF and derivates and agonists thereof; retinoic acid and derivates and agonists thereof; SANT-1 and derivates and agonists thereof; PDBu and derivates and agonists thereof; and LDN and derivates and agonists, such as the one or more factors selected from KGF, retinoic acid, SANT-1, PDBu and LDN. In one embodiment said step b-1) comprises culturing said cell population in a culture medium comprising one of more factors selected from KGF and derivates and agonists thereof and retinoic acid and derivates and agonists thereof, such as the one or more factors selected from KGF and retinoic acid, such as both KGF and retinoic acid. In one embodiment, said step b-1) comprises culturing said cell population in a culture medium comprising KGF or derivates or agonists thereof; retinoic acid or derivates or agonists thereof; SANT-1 or derivates or agonists thereof; PDBu or derivates or agonists thereof; and LDN or derivates or agonists thereof, such as culture medium comprising KGF, retinoic acid, SANT-1, PDBu and LDN. It will be appreciated that the culture medium may comprise a mixture of a factor and its derivative and/or agonist.

In one embodiment, said culture medium in step b-1) comprises 25 to 75 ng/ml KGF or derivate or agonist thereof, such as 50 ng/mL KGF or derivate or agonist thereof. In one embodiment said medium in step b-1) comprises approximately from 25 to 75 ng/ml of KGF, such as approximately 50 ng/ml of KGF. In one embodiment said medium in step b-1) comprises approximately from 1 to 3 μM of RA, such as approximately 2 μM of RA. In one embodiment said medium in step b-1) comprises approximately from 0.10 to 0.50 μM of SANT-1, such as approximately 0.25 μM of SANT-1. In one embodiment said medium in step b-1) comprises approximately from 250 to 750 nM of PDBu, such as approximately 500 nM of PDBu.

In one embodiment said medium in step b-1) comprises approximately from 100 to 300 nM of LDN, such as from 100-250 nM or LDN, such as 150-250 nM LDN, such as approximately 200 nM of LDN.

In one embodiment, step b-1) of the method as disclosed herein comprises culturing the cell population in a culture medium which comprises approximately 50 ng/ml KGF, approximately 2 μM retinoic acid, approximately 0.25 μM SANT-1, approximately 500 nM PDBu, and approximately 200 nM LDN.

In another embodiment of the method as disclosed herein, the method comprises, after the steps a)-c), the steps a+1)-c+1) of a+1) providing a cell population of pancreatic progenitor cells generated in step c;
b+1) culturing said cell population of pancreatic progenitor cells under conditions permissive of differentiation into endocrine progenitor cells; and
c+1) thereby generating a population of endocrine progenitor cells, such as endocrine progenitor cells characterized by the expression of NEURO D1, such as by expression of NKX6.1 and NEURO D1.

In one embodiment there is provided a method as disclosed herein, wherein prior to step i) the method comprises the steps
a−1) providing a cell population of primitive gut tube cells, such as primitive gut tube cells characterized by expression of HNF1β and/or HNF4α;
b−1) culturing said cell population of primitive gut tube cells for no more than approximately 54 hours under conditions permissive of differentiation into posterior foregut cells;
c−1) thereby generating a population of posterior foregut cells, such as posterior foregut cells characterized by the expression of PDX1;
a) providing the cell population of posterior foregut cells generated in step c−1), such as posterior foregut cells characterized by expression of PDX1;
b) culturing said cell population of posterior foregut cells for no more than approximately 78 hours, such as no more than approximately 72 hours, under conditions permissive of differentiation into pancreatic progenitor cells;
c) thereby generating a cell population of pancreatic progenitor cells, such as pancreatic progenitor cells characterized by expression of both PDX1 and NKX6.1;
a+1) providing the cell population of pancreatic progenitor cells generated in step c;
b+1) culturing said cell population of pancreatic progenitor cells under conditions permissive of differentiation into endocrine progenitor cells; and
c+1) thereby generating a population of endocrine progenitor cells, such as endocrine progenitor cells characterized by the expression of NEUROD1, such as by expression of NKX6.1 and NEUROD1.

In one embodiment, said population of endocrine progenitor cells is further characterized by the expression of at least one of NKX6.1 and NGN3. The skilled person will appreciate that a different combination of two markers could equally well be used for the endocrine progenitor cells, such as NKX6.1 and NGN3, NKX6.1 and NEUROD1 or NKX6.1 and NGN3 in order to distinguish the cells from pancreatic progenitor cells. For example, the population of endocrine progenitor cells in step c+1) may be characterized by the expression of PDX1, NKX6.1 and NEUROD1; PDX1, NKX6.1 and NGN3; or PDX1, NKX6.1, NEUROD1 and NGN3. It will be appreciated that the expression of NGN3 or NEUROD1 in PDX1+/NKX6.1⁺ cells (in other words, cells positive for both PDX1 and NKX6.1) is indicative of pancreatic progenitor cells taking on endocrine progenitor cell fate. The skilled person appreciates that due to experimental limitation in staining protocols, the expression of NGN3 or NEUROD1 may be evaluated cells stained for PDX1 or NKX6.1. In one embodiment of the method as disclosed herein, the cell population of endocrine progenitor cells is step c+1) is characterized by expression of PDX1, NKX6.1 and NEUROD1; PDX1, NKX6.1 and NGN3; or PDX1, NKX6.1, NEUROD1 and NGN3.

In one embodiment of the method as disclosed herein, the cell population of pancreatic progenitor cells provided in step a+1) is characterized by expression of PDX1 and NKX6.1, in other words co-expression of PDX1 and NKX6.1. In another embodiment, said cell population of pancreatic progenitor cells in step a+1) is further characterized by expression of one of more of the markers PTF1A, SOX9 and HNF6, such as two of the markers PTF1A, SOX9 and HNF6, such as all three of PTF1A, SOX9 and HNF6. In another embodiment, said cell population of pancreatic progenitor cells in step a+1) is further characterized by expression of one or both of the markers PTF1A and SOX9.

In embodiment of the method as disclosed herein, the cell population in step b+1) is cultured approximately from 3 to 5 days, such as approximately from 3 to 4 days or approximately 4 to 5 days, such as approximately 4 days. In one embodiment, the cell population in step b+1) is cultured approximately 5 days. This culture time is considered sufficient to generate the population of endocrine progenitor cells of step c+1).

Similarly to what was described for step a)-c), the step a+1)-c+1) are carried out in a 2D culture on a 2D substrate. It will be appreciated that the discussion of 2D substrates in relation to step a)-c) is equally relevant for steps a+1)-c+1) and is not repeated here merely for the sake of brevity. Similarly to what was described for step a)-c), the step a−1)-c−1) are carried out in a 2D culture on a 2D substrate. It will be appreciated that the discussion of 2D substrates in relation to step a)-c) is equally relevant for steps a−1)-c−1) and is not repeated here merely for the sake of brevity. Thus, in one embodiment method as disclosed herein, the cells in step b+1) are cultured on a 2D substrate. Thus, in one embodiment method as disclosed herein, the cells in step b−1) are cultured on a 2D substrate.

In one embodiment, said cells are adherent to said 2D substrate. Said 2D substrate may comprises one or more components selected from the group consisting of laminins (LN) and fragments thereof, vitronectin and fragments thereof, fibronectin and fragments thereof, collagen and fragments thereof, gelatin and fragments thereof, functionalized silk (FN silk) and Matrigel™, such as selected from the group consisting of laminins (LN) and fragments thereof, vitronectin and fragments thereof, fibronectin and fragments thereof, collagen and fragments thereof, gelatin and fragments thereof and Matrigel™, such as selected from the group consisting of laminins (LN) and fragments thereof, vitronectin and fragments thereof, fibronectin and fragments thereof, collagen, gelatin and Matrigel™, such as selected from the group consisting of laminins (LN) and fragments thereof, vitronectin and fragments thereof, fibronectin and fragments thereof and Matrigel™ In one embodiment, said 2D substrate may be comprise or consists of laminins (LN) and fragments thereof, such as recombinantly produced laminins (LN) and fragments thereof.

In one embodiment of the method as disclosed herein, wherein said laminins (LN) and fragments thereof are selected from the group consisting of LN-521 and fragments thereof, LN-511 and fragments thereof, LN-332 and fragments thereof, LN-421 and fragments thereof, LN-121 and fragments thereof and LN-111 and fragments thereof;
such as the group consisting of LN-521 and fragments thereof, LN-511 and fragments thereof, LN-332 and fragments thereof, LN-421 and fragments thereof and LN-121 and fragments thereof;
such as the group consisting of LN-521 and fragments thereof, LN-511 and fragments thereof and LN-332 and fragments thereof;
such as the group consisting of LN-521 and fragments thereof or the group consisting of LN-511 and fragments thereof. In one embodiment, said laminins and fragments thereof are selected from the group consisting of LN-521, LN-511, LN-332, LN-421, LN-121 and LN-111; such as the group consisting of LN-521, LN-511, LN-332, LN-421 and LN-121, such as the group consisting of LN-521, LN-511 and LN-332; such as the group consisting of LN-521 and LN-511; such as wherein said laminins and fragments thereof are LN-521 or wherein said laminins and fragments thereof are LN-511.

In one embodiment, said fragment(s) thereof is/are E8 fragment(s). In one embodiment, said laminins and fragments comprise an E8 fragment of laminin, such as an E8 fragment selected from the group consisting an E8 fragment of LN-511, an E8 fragment of LN-521, an E8 fragment of LN-332, an E8 fragment of LN-421, an E8 fragment of LN-121 and an E8 fragment of LN-111; such as the group consisting of an E8 fragment LN-511, an E8 fragment of LN-521, an E8 fragment of LN-332, and E8 fragment of LN-421 and an E8 fragment of LN-121; such as the group consisting of an E8 fragment LN-511, an E8 fragment of LN-521 and an E8 fragment of LN-332; such as the group consisting of an E8 fragment of LN-511 and an E8 fragment of LN-521; such as an E8 fragment of LN-511 or an E8 fragment of LN-521.

As discussed above, “conditions permissive of differentiation” refers to conditions which allow cells to exhibit the characteristics of said cell type and may include combinations of cell culture media, presence and/or absence of extrinsic factors as well as timing thereof. Said factors, as well as their derivatives and agonists have been discussed in relation to step b) and b−1) above in detail and said discussion will not be repeated here for the sake of brevity only. Thus, in one embodiment there is provided a method wherein step b+1) comprises culturing said cell population in a culture medium comprising one of more factors selected from BTC and derivatives and agonist thereof, retinoic acid and derivates and agonists thereof, Alk5 inhibitor (such as Alk5i II) and derivates and agonists thereof, retinoic acid and derivates and agonists thereof, γ-Secretase Inhibitor (such as GSI-XX) and derivates and agonists thereof, GC-1 and derivates and agonists thereof, LDN and derivates and agonists thereof, retinoic acid and derivates and agonists thereof and SANT-1 and derivates and agonists thereof; such as one or more factors selected from BTC, Alk5 inhibitor (such as Alk5i II), γ-Secretase Inhibitor (such as GSI-XX), GC-1, LDN, retinoic acid and SANT-1.

Thus, in one embodiment there is provided a method wherein step b+1) comprises culturing said cell population in a culture medium comprising BTC or derivates or agonists thereof; Alk5 inhibitor (such as Alk5i II) or derivates or agonists thereof: γ-Secretase Inhibitor (such as GSI-XX) or derivates or agonists thereof; GC-1 orderivates or agonists thereof; LDN or derivatesor agonists thereof; retinoic acid or derivates or agonists thereof; and SANT-1 or derivates ior agonists thereof; such as BTC, Alk5 inhibitor (such as Alk5i II), γ-Secretase inhibitor (such as GSI-XX) GC-1, LDN, retinoic acid and SANT-1. Thus, in one embodiment there is provided a method wherein step b+1) comprises culturing said cell population in a culture medium comprising BTC and/or derivates and/or agonists thereof; Alk5 inhibitor (such as Alk5i II) and/or derivates and/or agonists thereof; γ-Secretase Inhibitor (such as GSI-XX) and/or derivates and/or agonists thereof; GC-1 and/or derivates and/or agonists thereof; LDN and/or derivates and/or agonists thereof; retinoic acid and/or derivates and/or agonists thereof; and SANT-1 and/or derivates and/or agonists thereof. It will be appreciated that the culture medium may comprise a mixture of a factor and its derivative and/or agonist. In one embodiment of the method as disclosed herein, step b+1) comprises culturing said cell population in a culture medium comprising BTC, Alk5i II, GSI-XX, GC-1, LDN, retinoic acid and SANT-1. In particular, in one embodiment there is provided a method wherein step b+1) comprises culturing said cell population in a culture medium comprising at least Alk5 inhibitor or a derivate or agonist thereof and γ-Secretase Inhibitor and derivates and agonists thereof, such as Alk5i II and GSI-XX.

BTC, also known as betacellulin, is a member of the EGF family of growth factors and induces differentiation of β-cell, but also of other cell types. As used herein, the term “BTC and derivates and agonists thereof” refers to EGFR ligands/EGF-family growth factors.

An example of an ALK5 inhibitor is Alk5i II (ALK5 Inhibitor II), which is a cell permeable, selective inhibitor of the TGF-β type 1 activin like kinase receptor ALK5. Non-limiting examples of ALK5 inhibitors include Alk5i II, LY2157299, LY364947, Repsox, SB525334, A83-01, GW788388, LY-2109761, SB-505124 and D4476. In one embodiment, said Alk5 inhibitor or derivate or agonist thereof is selected from the group consisting of Alk5i II, LY2157299, LY364947, Repsox, SB525334, A83-01, GW788388, LY-2109761, SB-505124 and D4476.

GSI-XX, also known as γ-Secretase Inhibitor XX, is cell-permeable dibenzazepine compound that inhibits γ-Secretase. γ-Secretase is a multi-subunit protease complex, itself an integral membrane protein, that cleaves single-pass transmembrane proteins at residues within the transmembrane domain. Non limiting examples of γ-Secretase inhibitors include GSI-XX, DAPT, RO4929097, YO-01027, BMS-906024, AB42-IN-2, LY-411575 and MK-0752. Thus, in one embodiment, said γ-Secretase inhibitor is selected from GSI-XX, DAPT, RO4929097, YO-01027, BMS-906024, AB42-IN-2, LY-411575 and MK-0752.

GC-1 is a thyroid hormone receptor (TR) agonist and is more potent than the thyroid hormone T3. Thyroid hormone T3 is important for the development of β-cells.

As used herein, the term “GC-1 and derivates and agonists thereof” refers to GC-1, T3 and T4. Thus in one embodiment, said GC-1 and derivates and agonists thereof is selected from the group consisting of GC-1, T3 and T4.

In one embodiments, said culture medium in step b+1) is a culture medium suitable for culture of pancreatic progenitor cells under conditions permissive of differentiation into endocrine progenitor cells. Non limiting examples of stage 5 (S5) medium are as defined in the present Examples. The skilled person appreciates that other suitable media may be used. Said culture medium in step b+1) may be supplemented by other factors a specified herein.

In one embodiment said medium in step b+1) comprises approximately from 10 to 30 ng/ml of BTC, such as approximately 20 ng/ml of BTC.

In one embodiment said medium in step b+1) comprises approximately from 5 to 15 μM of Alk5i II, such as approximately 10 μM of Alk5i II.

In one embodiment said medium in step b+1) comprises approximately from 50 to 150 nM of GSI-XX, such as approximately 100 nM of GSI-XX.

In one embodiment said medium in step b+1) comprises approximately from 0.5 to 1.5 μM of GC-1, such as approximately 1 μM of GC-1.

In one embodiment said medium in step b+1) comprises approximately from 50-150 nM of LDN, such as from 75-125 nM or LDN, such as approximately 100 nM of LDN.

In one embodiment said medium in step b+1) comprises approximately from 50 to 150 nM of RA, such as approximately 100 nM of RA.

In one embodiment said medium in step b+1) comprises approximately from 0.10 to 0.50 μM of SANT-1, such as approximately 0.25 μM of SANT-1. In one embodiment, step b+1) of the method as disclosed herein comprises culturing the cell population in a culture medium which comprises approximately 20 ng/ml of BTC, approximately 10 μM of Alk5i II, approximately 100 nM of GSI-XX, approximately 1 μM of GC-1, approximately 100 nM of LDN, approximately 100 nM of RA and approximately 0.25 μM of SANT-1.

As discussed above, in one embodiment there is provided a method for the generation pancreatic islet-like cell aggregates in vitro as disclosed herein, wherein said population of endocrine progenitor cells, such as endocrine progenitor cells characterized by the expression of NEUROD1. In one embodiment, said EP cells are characterized by the expression of NGN3. In one embodiment, said EP cells are characterized by the expression of NGN3 and NEUROD1. In another embodiment, said EP cells are characterized by the expression of NKX6.1 and NEUROD1. In one embodiment, said EP cells are further characterized by the expression of at least one of PDX1 and NGN3.

In one embodiment, in said step b+1) said cell population of pancreatic progenitor cells is cultured for approximately from 3 to 5 days, such as approximately from 3 to 4 days or approximately 4 to 5 days, such as approximately 4 days or such as approximately 5 days. In one embodiment said pancreatic progenitor cells are cultured on a 2D substrate at least until the generation endocrine progenitor cells in step c+1).

The cell may during the differentiation protocol be cultured on a 2D substrate, such as adherent on a 2D substrate. It will be appreciated that the discussion relation to the identity of the 2D substrate above, is equally relevant in the contexts of steps a)-c+1) and will not be repeated here merely for the sake of brevity.

It will be appreciated that the endocrine progenitor cells obtained in step c+1) may be differentiated further into pancreatic monohormonal β-cells. The present inventors have found that transfer for cells from 2D culture conditions to 3D culture conditions is beneficial at this step.

In one embodiment of said method, the cells are not transferred from culture on a 2D substrate to culture on a 3D substrate prior to exhibiting expression of markers characteristic of endocrine progenitor cells. Endocrine progenitor cells are characterized by the expression of PDX1, NKX6.1 and at least one of NEUROD1 and NGN3, as discussed above.

In one embodiment, there is provided a method as disclosed herein, said method, after steps a+1)-c+1), further comprising steps i)-v) as defined herein comprising transferring said population of endocrine progenitor cells, such as endocrine progenitor cells characterized by the expression of NKX6.1 and NEUROD1, from culture on a 2D substrate to 3D culture conditions; culturing said population of endocrine progenitor cells under conditions permissive of differentiation pancreatic monohormonal β-cells; and leading to generating a population of pancreatic monohormonal β-cells, such as pancreatic monohormonal β-cells characterized by the expression of insulin. Said monohormonal β-cells may further express at least one of NKX6.1, PDX1 and NEUROD1.

As discussed in detail above, the 3D culture conditions allow for self-aggregation of the cells.

In one embodiment, steps i)-v) follow steps a+1)-c+1), such as immediately follow steps a+1)-c+1). t will be appreciated that endocrine progenitor cells may be cryopreserved for a desired period of time before steps i)-v) are performed. In this case, the steps i)-v) follow a cryopreservation step and subsequent recovery of cryopreserved cells. The skilled person is familiar with the process of recovery of cryopreserved cells, which in general terms comprises rapidly thawing cells in a water bath at 37° C., removing cells from the freeze-medium by gentle centrifugation and/or dilution with growth medium, and seeding the cells in a culture vessel in complete growth medium. Thus, in one embodiment, steps i)-v) follow cryopreservation and subsequent recovery of EP cells obtained in c+1). In one embodiment, step iv) comprises culturing said population of endocrine progenitor cells for approximately 3 weeks or longer, such as for approximately from 3 to 5 weeks, such as for approximately 4 weeks.

As used herein, the term “monohormonal” refers to cells that express only one type of hormone. For example, to monohormonal β-cells that expression only insulin and do not express other hormones that are expressed by pancreatic islet cells, such as glucagon or somatostatin. As used herein, the term “polyhormonal” refers to cells which express at least two different hormones.

Monohormonal β cell in vivo are monohormonal cell and it is beneficial that the population obtained by the inventive method exhibits the properties of natural β-cell in vivo, or endogenous β-cell in vivo, such as properties of healthy natural β-cell in vivo, or healthy endogenous β-cell in vivo. Thus, in one embodiment of the present method as disclosed herein, said population of monohormonal β-cells generated in step v) is monohormonal. In particular, said population of monohormonal β-cells generated in step v) does not express glucagon or somatostatin. In particular, said population of monohormonal β-cells generated in step v) does not express glucagon and somatostatin.

It will be appreciated that the increased proportion and/or number of EP cells obtained by the method according to steps a−1) to c+1), compared to methods of the prior art wherein step b) of culturing said cell population of posterior foregut cells for approximately 96 hours or more under conditions permissive of differentiation into pancreatic progenitor cells, may be translated into higher quality of pancreatic islet-like cell aggregates, such as comprising a higher proportion of monohormonal β-cells as defined herein. Also, said islet-like cell aggregates may comprise low percentage polyhormonal cells, low percentage monohormonal cells which are not β-cells and/or low percentage proliferating cells.

In one embodiment as disclosed herein, a method is provided wherein in step c+1) more than approximately 30%, such as more than approximately 40%, 40%, such as more than approximately 45%, such as more that approximately 50% of the total cell population are endocrine progenitor cells, such as endocrine progenitor cells characterized by the expression of NKX6.1 and at least NEUROD1. As discussed and exemplified above other combinations of two or more of the markers PDX1, NKX6.1, NEUROD1 and NGN3 can be used as characteristic of endocrine progenitor cells. In one embodiment, in step c+1) more than approximately 30%, such as more than approximately 40%, such as more than approximately 45%, such as more than approximately 50% of the total cell population are endocrine progenitor cells, such as endocrine progenitor cells characterized by the expression of NKX6.1 and NEUROD1.

In one embodiment there is provided a method as disclosed herein, wherein the number of endocrine progenitor cells in step c+1) is higher compared to the number of endocrine cells obtained using the corresponding method in which step b) of culturing said cell population of posterior foregut cells under conditions permissive of differentiation into pancreatic progenitor cells is for approximately 24 hours or less and/or is for approximately 96 hours or more. Thus, the time of culture at step b) according to the present invention leads to an increased number of EP cells. As shown in the appended examples, the number of EP cells obtained by the method as disclosed herein, compared to a method with a longer or shorter culture time in step b), is significantly higher. In particular, this is surprising and unexpected as the number of PP cells is lower compared to the number of PP cells when step b) is approximately 96 hours or more.

In one embodiment said method results in at least approximately 10%, such as at least approximately 15%, such as at least approximately 20%, such as at least approximately 30%, such as at least approximately 40%, such as at least 50%, such as at least 60% more endocrine progenitor cells than the corresponding method in which step b) of culturing said cell population of posterior foregut cells under conditions permissive of differentiation into pancreatic progenitor cells is for approximately 24 hours or less and/or is for approximately 96 hours or more.

In one particular embodiment of the method as disclosed herein, said number of endocrine progenitor cells in step c+1) is higher when step b−1) of culturing said cell population of primitive gut tube cells under conditions permissive of differentiation into posterior foregut cells does not exceed 54 hours in order to obtain a population of posterior foregut cells, compared to the number of endocrine cells obtained using the corresponding method in which step b−1) of culturing said cell population of primitive gut tube cells under conditions permissive of differentiation into posterior foregut cells is for approximately 56 hours of more. In particular, the combination of a culture time of step b−1) and the culture time of step b) is considered to be particularly beneficial in terms of number of EP cells obtained in step c+1).

This effect is demonstrated in the appended examples and FIG. 4 in particular, wherein the number endocrine progenitor cells is scored by co-expression of NKX6.1 and NEUROD1. Importantly the effect is shown in cultures of cells from different human stem cell lines, including human ESC lines and IPSC lines.

As explained above, it will be appreciated that the increased proportion and/or number of EP cells obtained by the present method, compared to methods of the prior art wherein step b) of culturing said cell population of posterior foregut cells for approximately 96 hours or more under conditions permissive of differentiation into pancreatic progenitor cells, may be translated into a higher proportion of monohormonal β-cells as defined herein. It will be appreciated that the increased proportion and/or number of EP cells obtained by the present method, compared to methods of the prior art wherein step b-1) is 56 hours or more and step b) is 96 hours or more, may be translated into a higher proportion of monohormonal β-cells as defined herein.

In one embodiment of the method as disclosed herein, there is provided a method wherein in step v) more than approximately 40%, such as approximately from 40 to 50%, such as approximately from 40 to 60%, such as approximately from 40 to 70%, of the total cells in the pancreatic islet are monohormonal β-cells, such as monohormonal β-cells characterized by the expression of insulin. In one embodiment, said method results in at least 30%, such as at least approximately 35%, such as at least approximately 40%, more monohormonal β-cell, such as monohormonal β-cells characterized by the expression of insulin, than the corresponding method, in which step b) of culturing said cell population of posterior foregut cells is for approximately 96 hours under conditions permissive of differentiation into pancreatic progenitor cells. In one embodiment, said method results in at least 30%, such as at least approximately 35%, such as at least approximately 40%, more monohormonal β-cell, such as monohormonal β-cells characterized by the expression of insulin, than the corresponding method, in which step b−1) is hours or more 56 hours and step b) is 96 hours or more. Said monohormonal β-cells may be characterized by insulin and at least one of NKX6.1, PDX1 and NEUROD1, such as by expression of insulin and NKX6.1.

In one embodiment, said monohormonal β-cells express insulin and is further characterized by the expression of NKX6.1, PDX1 and/or NEUROD1. In one embodiment, said monohormonal β-cells are characterized by the expression of insulin and NKX6.1; insulin and PDX1; or insulin and NEUROD1.

In one embodiment, said monohormonal β-cells are characterized by the expression of insulin and two of NKX6.1, PDX1 and NEUROD1; such as insulin, NKX6.1 and PDX1; or insulin, NKX6.1 and NEUROD1; or insulin, PDX1 and NEUROD1. In one embodiment, said monohormonal β-cells are characterized by the expression of insulin, PDX1, NKX6.1 and NEUROD1. In one particular embodiment, said method results in at least 2 times more pancreatic monohormonal β-cells, such as pancreatic monohormonal β-cells characterized by expression of insulin, than monohormonal pancreatic q-cells characterized by expression of glucagon. Thus, the islet-like cell aggregates obtained by the present method comprise least 2 times more pancreatic monohormonal β-cells than α-cells.

The skilled person appreciates that monohormonal pancreatic β-cells not only express insulin together with markers characteristic for mature pancreatic β-cells, but also that said monohormonal β-cells are functional pancreatic β-cells and thus are able to respond to glucose stimulation. The outcome of glucose stimulation may be scored by insulin production and/or by expression of c-peptide. C-peptide is released at the same time as insulin and for each molecule of insulin produced there is a molecule of C-peptide produced by β-cells. C-peptide does not itself influence blood sugar, however C-peptide is a useful marker of insulin production because C-peptide tends to remain in the blood longer than insulin.

Thus, in one embodiment, there is provided a method as described herein, wherein said monohormonal β-cells, such as said monohormonal β-cells of said islet-like cell aggregates, are functional pancreatic β-cells, such as functional pancreatic β-cells as scored by expression of C-peptide upon glucose stimulation.

The aim of the present method is to provide pancreatic islet-like cell aggregates comprising monohormonal β-cells, such as human pancreatic islet-like cell aggregates comprising monohormonal β-cells, by means of in vitro differentiation.

The cells of origin may be pluripotent cells, such as a cell line of pluripotent cells, for example embryonic stems cells or induced pluripotent stem cells. Thus, the cells may be from an established cell line, or alternatively, said cells may be primary cells derived directly from a patient, such as patient specific cells. Thus, in one embodiment of the method as disclosed herein, the cell population in step a) or a−1) or a+1) is derived from a culture of pluripotent stem cells, such as a culture of induced pluripotent stem (IPS) cells or a culture of embryonic stem (ES) cells. In one embodiment, the cell population in step a) or a−1) or a+1) is a population of primary cells derived directly from a patient. Alternatively, the cell population in step a) or a−1) or a+1) may be derived from a culture of multipotent cells, for example a cell line, which have been restricted to the endodermal lineage. Said cells may be human cells.

In one embodiment, said cell population in step a) or a−1) is a mammalian cell population, such as human cell population. Consequently, the same applies to the cell population in step i). Thus, the cell population in step i) may be derived from a culture of pluripotent stem cells, such as a culture of induced pluripotent stem (IPS) cells or a culture of embryonic stem (ES) cells. The cell population in step i) may be a population of primary cells derived directly from a patient or be derived from a culture of multipotent cells, for example a cell line, which have been restricted to the endodermal lineage. Said cells may be human cells.

As used herein, the term “derived from” refers to the origin of the cell and cells in step i) may be derived via one or several differentiation step (for example, one of several of a−1-c−1); a-c); and a+1-c+1) as defined herein) prior to step i).

It is known in the art that stem cells are undifferentiated cells defined by their ability, at the single cell level, to both self-renew and differentiate. Stem cells may produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm). Stem cells also give rise to tissues of multiple germ layers following transplantation and contribute substantially to most, if not all, tissues following injection into blastocysts.

Stem cells are classified by their developmental potential as: (1) totipotent, meaning able to give rise to all embryonic and extra embryonic cell types; (2) pluripotent, meaning able to give rise to all embryonic cell types; (3) multipotent, meaning able to give rise to a subset of cell lineages but all within a particular tissue, organ, or physiological system; (4) oligopotent, meaning able to give rise to a more restricted subset of cell lineages than multipotent stem cells; and (5) unipotent, meaning able to give rise to a single cell lineage.

As explained above, differentiation is the process by which an unspecialized (“uncommitted”) or less specialized cell acquires the features of a specialized cell such as, for example, a nerve cell or a muscle cell. A differentiated cell or a differentiation-induced cell is one that has taken on a more specialized (“committed”) position within the lineage of said cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. “De-differentiation” refers to the process by which a cell reverts to a less specialized (or committed) position within the cell lineage.

It is noted that the technical teaching of the present invention can be put into practice using any human pluripotent embryonic stem cells, including human pluripotent embryonic stem cells that were derived without destruction of human embryos, such as from parthenogenetically activated oocytes. In particular embodiment, said cell population in step i) is derived from a human embryonic stem cell population. In one embodiment, said cell population in step i) is derived from human embryonic stem cell populations which were obtained without destruction of human embryos. In particular embodiment, said cell population in step a) or step a−1) or step a+1) is derived from a human embryonic stem cell population. In one embodiment, said cell population in step a) or step a−1) or step a+1) are derived from human embryonic stem cell populations which were obtained without destruction of human embryos.

In one particular embodiment, said cell population in step a), a−1), a+1) or i) is derived from a human embryonic stem cell population, such as human embryonic stem cell population selected from the group of embryonic stem cell lines consisting of HS980 cells, H1 cells and H9 cells, such as the group of embryonic stem cell lines consisting of H1 cells and HS980 cells, or the group of embryonic stem cell lines consisting of H1 and H9 cells, or the group of embryonic stem cell lines consisting of HS980 cells and H9 cells. In one embodiment said cells are H1 cells. In one embodiment said cells are H9 cells. In one embodiment said cells are HS980 cells. In one embodiment, said human embryonic stem cell population is a population obtained without destruction of embryos. Merely for the purpose of compliance with European patent practice, embodiments relating to HS980, H1 and/or H9 cells recited above are to be regarded as reference examples for the European jurisdiction.

In one particular embodiment, said cell population in step a), a−1), a+1) or i) is derived from IPS cells, such as human IPS cells.

In one particular embodiment, said IPS cells are selected from the group consisting of patient derived IPS cells and IPS cell lines. In one particular embodiment, said such as IPS cell lines is CTRL-7-II (C7). C7 was described in Kele M et al 2016.

As discussed above, said method may further comprise the step of cryopreservation of cells, in particular it may be suitable to cryopreserve endocrine progenitor cells. Thus in one embodiment, there is provided a method comprising the step of cryopreservation of endocrine progenitor cells. In one embodiment, said cryopreservation is of endocrine progenitor cells generated in step c+1). In one embodiment, said method as disclosed herein comprises cryopreservation of EP cells prior to step i). In one embodiment, the population of EP cells provided in step i) is a cryopreserved population of EP cells or a population of EP cells that has previously been cryopreserved. As is shown in the appended Examples, the cryopreservation does not negatively impact the generation of said islet-like cell aggregates or of monohormonal β-cells.

In a second aspect of the present disclosure, there is provided isolated pancreatic islet-like cell aggregates or an isolated population of pancreatic islet-like cell aggregates obtainable by the method as described herein. Also provided are cells obtainable from said pancreatic islet-like cell aggregates as discussed in the following third aspect. In particular, said pancreatic islet-like cell aggregates comprise monohormonal β-cells. As explained in the context of the first aspect, it is highly desirable that the pancreatic islet-like cell aggregates obtained in vitro mimic the characteristics of pancreatic islets in vivo, both in terms of distribution of cell types present herein and also in their functional properties. The skilled person will appreciate that the characteristics of the

In particular, said isolated pancreatic islet-like cell aggregates or isolated population of pancreatic islet-like cell aggregates are characterized by that they exhibit desirable properties of high number of monohormonal β-cells, a desired number of monohormonal α-cells, low number of polyhormonal cells (including low number of polyhormonal β-cells and low number of polyhormonal α-cells), low number of non-endocrine cells, and low number of proliferating cells.

Thus, in one embodiment, said wherein said isolated pancreatic islet-like cell aggregates comprise at least approximately 25%, such as at least approximately 30%, such as at least approximately 35%, such as at least approximately 40%, such as at least approximately 45%, such as at least approximately 50% such as at least approximately 55%, such as at least approximately 70% monohormonal β-cells. In one embodiment said pancreatic islet-like cell aggregates comprise approximately from 25 to 70%, such as approximately from 30 to 70%, such as approximately from 40 to 70%, such as approximately from 40 to 60% monohormonal β-cells.

In one embodiment, said isolated pancreatic islet-like cell aggregates comprise at least 40%, such as at least 45%, such as at least 50% such as at least 55% β-cells, such as at least 60% β-cells, such as at least 65%, such as at least 70% β-cells monohormonal β-cells. In one embodiment, said monohormonal β-cells are characterized by INSULIN expression.

In one embodiment, said pancreatic islet-like cell aggregates comprise at most approximately 20%, such as at most approximately 18%, such as at most approximately 16%, such as at most approximately 13%, such as at most approximately 10% monohormonal alpha cells. In one embodiment, said monohormonal alpha cells are characterized by GLUCAGON expression. In one embodiment, said isolated islet-like cell aggregates in comprise monohormonal β-cells and alpha cells and comprise less than approximately 5%, such a less than approximately 4, 3, 2 or 1%, of cells selected form the group consisting of delta cells, acinar cells, ductal cells and activated stellate cells. In one embodiment of said method, said islet-like cell aggregates in v) comprise at most approximately 5%, such as at most approximately 4, 3, 2 or 1% polyhormonal cells. In one embodiment of said method, said islet-like cell aggregates in v) comprise at most approximately 5%, such as at most approximately 4, 3, 2 or 1% non-endocrine cells. Thus, in one embodiment said isolated pancreatic islet-like cell aggregates comprise, or said isolated population comprises pancreatic islet-like cell aggregates which comprise, at least approximately 25%, such as at least approximately 30%, such as at least approximately 35%, such as at least approximately 40%, such as at least approximately 45%, such as at least approximately 50%, such as at least approximately 55%, such as at least approximately 60%, such as at least approximately 65%, such as at least approximately 70% monohormonal β-cells. In one embodiment said isolated pancreatic islet-like cell aggregates comprise, or said isolated population comprises pancreatic islet-like cell aggregates which comprise, approximately from 25 to 70%, such as from 30 to 70%, such as from 30 to 70% monohormonal β-cells, such as from 35 to 70%, from 35 to 70%, such as from 40 to 70%, such as from 45 to 70%, such as from 45 to 65%, such as from 45 to 60%, such as from 45 to 55%, such as approximately 50% monohormonal β-cells. In one embodiment said pancreatic islet-like cell aggregates comprise, or said population comprises pancreatic islet-like cell aggregates which comprise, approximately from 35 to 65%, such as from 40 to 65%, such as from 40 to 60% monohormonal β-cells.

In one embodiment, said pancreatic islet-like cell aggregates comprise, or said population comprises pancreatic islet-like cell aggregates which comprise, approximately from 7 to 25%, such as from 7 to 20%, from 10 to 20%, such as from 15 to 20%, such as approximately 20% monohormonal α-cells.

In one embodiment said pancreatic islet-like cell aggregates comprise, or said population comprises pancreatic islet-like cell aggregates which comprise, at most approximately 10%, such as at most approximately 7%, such as at most approximately 6%, such as at most approximately 5%, such as at most approximately 4%, such as at most approximately 3%, such as at most approximately 2%, such as at most approximately 0.5%, such as at most approximately 0.3%, such as at most approximately 0.1%, poly hormonal α-cells.

In one embodiment said pancreatic islet-like cell aggregates comprise, or said population comprises pancreatic islet-like cell aggregates which comprise, at most approximately 10%, such as at most approximately 7%, such as at most approximately 6%, such as at most approximately 5%, such as at most approximately 4%, such as at most approximately 3%, such as at most approximately 2%, such as at most approximately 0.5%, such as at most approximately 0.3%, such as at most approximately 0.1%, poly hormonal β-cells.

In one embodiment, said pancreatic islet-like cell aggregates comprise, or said population comprises pancreatic islet-like cell aggregates which comprise, less than 5%, such as less than 4%, such as less than 3%, such as less than 1% delta cells.

In one embodiment said pancreatic islet-like cell aggregates comprise, or said population comprises pancreatic islet-like cell aggregates which comprise, at most approximately 5%, such as at most approximately 4%, such as at most approximately 3%, such as at most approximately 2%, such as at most approximately 1%, such as at most approximately 0.5%, such as at most approximately 0.1%, proliferating cells, such as proliferating cells with express Ki-67.

In one embodiment, said isolated pancreatic islet-like cell aggregates in vitro are scored at the end of S6, such on day 38-42 of culture as described herein, such as at day 38, 39, 40, 41, 42 or later.

It will be appreciated said isolated pancreatic islet-like cell aggregates, population thereof or cells therefrom may be useful in therapy, such as cell replacement therapy, as well as in drug development or other research applications. In particular, it will be appreciated that it is advantageous to provide a population which exhibit high percentage or high fraction of the desired cell type, without having any need for additional selection or sorting of cells. To clarify, term “isolated” in relation to the isolated pancreatic islet-like cell aggregates, population thereof or cells from the pancreatic islet-like cell aggregates, refers to that the cells are removed (in other words isolated) from their natural environment, such as environment in the body. It will be appreciated that the population of isolated pancreatic islet-like cell aggregates, population thereof or cells from the pancreatic islet-like cell aggregates as disclosed herein may be a part of or whole a cell aggregate formed during cell culture. Said aggregate (also referred to as islet-like cell aggregates) may comprise for example pancreatic alpha cells and/or delta cells or cells of the pancreatic alpha and/or delta cell lineage(s) in addition to pancreatic β-cells.

Thus, in one embodiment said isolated pancreatic islet-like cell aggregates or population of pancreatic islet-like cell aggregates is provided, wherein the cells comprising said islet-like cell aggregates have not been subject to enrichment for a desired phenotype, such as have not been subject to enrichment by manual intervention or by means of a machine (in other words automated sorted). In one embodiment, said cells have not been subject to enrichment prior to forming the 3D structures in step iii). Thus, in one embodiment of isolated pancreatic islet-like cell aggregates or isolated population of pancreatic islet-like cell aggregates, the cells comprising said islet-like cell aggregates have not been subject to enrichment for a desired phenotype, such as have not been subject sorting for a desired phenotype, such as sorting based on desired marker expression or such as sorting for a desired phenotype based on FACS. The skilled person appreciates that selecting/sorting may be based on the presence of a desired phenotype (for example based on marker expression) or on the absence of an undesired phenotype, in which case the undesired cells are removed from a population and thus the population is enriched for the cells exhibiting the desired phenotype. In one embodiment, said cells have not been subject to sorting prior to forming the 3D structures in step iii). For the sake of clarity, the term “enrichment” as used herein refers to enrichment of cells by (manual or by means of a machine) intervention, for example sorting of cells based on their properties, and not to naturally occurring processes in the cell culture. Thus in one embodiment, the cells comprising said islet-like cell aggregates (in other words cells of said islet-like cell aggregates) have not been subject sorting for a desired phenotype, such as sorting based on desired marker expression. In one embodiment, the cells comprising said islet(s) have not been subject to sorting for a desired phenotype based on FACS. In one embodiment, the cells comprising said islet(s) have not been subject to enrichment by removal of an undesired phenotype, for example based on marker expression, for example by means of FACS.

The inventive islet-like cell aggregates is characterized by a high percentage of cells with desirable properties obtained by means of the differentiation method as such. Thus, in one embodiment of the present aspect, there is/are provided isolated pancreatic islet-like cell aggregates wherein more than approximately 40%, such as approximately from 40 to 60%, such as approximately from 40 to 50% of the total cell population of said islet(s) are monohormonal β-cells, such as monohormonal β-cells characterized by the expression of insulin. In one embodiment, said population comprises at least 2 times, such as 3, 4, 5 times, more monohormonal pancreatic β-cells characterized by expression of insulin, than monohormonal pancreatic q-cells characterized by expression of glucagon.

Said monohormonal β-cells may be characterized as described above, for example by the expression of insulin and at least one or more, such as two, of the markers NKX6.1, PDX1 and NEUROD1. Said monohormonal β-cells may be characterized by the expression of insulin and NKX6.1, such as characterized by the expression of insulin, NKX6.1 and at least one of PDX1 and NEUROD1. In one embodiment, said monohormonal β-cells may be characterized the expression of insulin, NKX6.1, PDX1 and NEUROD1. In one embodiment, said monohormonal β-cells may comprise part of cell aggregates, such as cell aggregates further comprising pancreatic monohormonal alpha and/or delta cells.

In a related third aspect, there are provided cells obtained from isolated pancreatic islet-like cell aggregates as defined herein obtained by dissociation of said pancreatic islet(s). Thus, a population of such cells is provided. For the sake of brevity, such cells are referred to as “cells therefrom” in the present context.

As mentioned above, it is envisioned that the isolated pancreatic islet or isolated pancreatic islet-like cell aggregates or cells obtained therefrom as disclosed herein will be useful in the treatment and/or prevention of diabetes. Thus, in a third aspect of the present disclosure, there is provided isolated pancreatic islet-like cell aggregates, a population of isolated pancreatic islet-like cell aggregates or cells obtained therefrom as disclosed herein, for use in therapy (in other words for use as a medicament).

It will be appreciated that said islet-like cell aggregates or cells therefrom can be transplanted into a patient in need thereof for the purpose of cell replacement therapy. Said replacement therapy may be for the purpose of providing pancreatic β-cells to a patient who does not have endogenous β-cells or only has non-functional β-cells, or to a patient who needs more pancreatic β-cells as his or her endogenous population is either deminished or less functional than required for a healthy patient status.

Said islet-like cell aggregates or cells may be derived from a donor, thus be allogeneic. For example, said islet-like cell aggregates or cells may be derived from a relative or from a unrelated donor or derived from an established cell line, such as a stem cell line with which has the capacity to develop along the pancreatic β-cell lineage. Non-limiting examples include hES cell lines, non-embryonic stem cell lines (for example multipotent, oligopotent or unipotent cell lines) which have the potential to develop along the endocrine lineage, iPS cell lines and a cell line derived from iPS cells with the capacity to develop along the pancreatic β-cell lineage. Said cells may also be endogenous to the patient, for example derived from iPS cells obtained from the patient or other patient derived cells with the capacity to develop along the pancreatic β-cell lineage.

It is considered that the islet-like cell aggregates or cells obtained by the method as disclosed herein will be beneficial for use in therapy as the percentage of total cells of the desired cell type is higher than in populations obtained by culture methods previously described. Hence, the islet-like cell aggregates or cells obtained by the disclosed method, will to at least a lower extent compared to the islet-like cell aggregates or cells generated by methods known in the prior art, if at all, need to be subjected to cell sorting or similar stressful and potentially damaging techniques in order to obtain homogenous populations with high percentage of the desired cell type. Thus, it is expected that treatment according to the present disclosure, such as transplantation (for example in the form om cell replacement therapy), may be performed with heathier and more viable islet or cell populations compared to those generated by methods known in the prior art, and that the inventive islet-like cell aggregates or cell populations contain a lower percentage damaged and/or unhealthy cells, which is considered to be beneficial to patients in terms a less potential adverse side effects and better clinical treatment outcomes.

In a fourth aspect, there is provided isolated pancreatic islet-like cell aggregates, an isolated population pancreatic islet-like cell aggregates or cells therefrom as disclosed herein, for use in the treatment, prevention and/or amelioration of diabetes, such as type 1 or type 2 diabetes. In one embodiment, said diabetes is type 1 diabetes. In one embodiment, said type 2 diabetes is insulin deficient type 2 diabetes.

In one embodiment, there is provided isolated pancreatic islet, an isolated population pancreatic islet-like cell aggregates or cells therefrom as disclosed herein for use in the treatment in therapy, wherein said isolated pancreatic islet, population of isolated pancreatic islet-like cell aggregates or cells obtained therefrom as disclosed herein have been generated by a method as defined herein.

In one embodiment, there is provided isolated pancreatic islet-like cell aggregates, an isolated population pancreatic islet-like cell aggregates or cells therefrom for use in therapy, in other words for use as a medicament, wherein said use comprises the steps of generating cells of the isolated pancreatic islet-like cell aggregates, an isolated population pancreatic islet-like cell aggregates or cells obtained therefrom according to the method as defined herein; and administering a therapeutically effective amount of said islet-like cell aggregates or cells to a patient.

In one embodiment, said use further comprises isolation of cells, from said patient, such as of cells for generation of IPS cells from said patient or of stem cells, and using said cells for generating islet-like cell aggregates or cells according to the method as defined herein.

In one embodiment, there is provided isolated pancreatic islet-like cell aggregates or cells obtained therefrom for use in the treatment, prevention and/or amelioration of diabetes, such as type 1 or type 2 diabetes, wherein said islet-like cell aggregates or cells have been generated by a method as defined herein.

In another embodiment there is provided an isolated pancreatic islet-like cell aggregates or cells therefrom for use in the treatment, prevention and/or amelioration of diabetes, such as type 1 or type 2 diabetes, wherein use comprises the steps of generating said islet-like cell aggregates or cells according to the method as defined herein; and administering a therapeutically effective amount of said cells to a patient. In one embodiment, said islet-like cell aggregates may be administered in the form of pancreatic islet-like cell aggregates, in other words in the form of cell aggregates, as defined above. Alternatively, said islet-like cell aggregates may be dissociated and the cells may be administered in the form a cell suspension, such as a single cell suspension. In one embodiment, said use further comprises isolation of cells, such as cells for generation of IPS cells from said patient or of stem cells, from said patient and using said cells for generating cells of said islet-like cell aggregates or cells therefrom, according to the method as defined herein. Similar to what discussed in relation to the third aspect other cell types are considered useful in this regard, including allogeneic and endogenous cells.

Thus, in one embodiment said use comprises the steps of generating isolated pancreatic islet-like cell aggregates according to the method as defined herein; and administering a therapeutically effective amount of said islet-like cell aggregates to a patient.

Thus, in one embodiment said use comprises the steps of generating isolated pancreatic islet-like cell aggregates according to the method as defined herein;
dissociating the pancreatic islet-like cell aggregates, and
administering therapeutically effective amount of said dissociated islet cells to a patient.

It will be appreciated that said cell replacement therapy may involve the transplantation of islet-like cell aggregates (cell aggregates) or cells obtained therefrom, which then produce insulin in vivo in the patient and have the ability to respond to glucose stimulation in the patient.

Thus in one embodiment, said use comprises transplantation of said islet-like cell aggregates for cells into a patient in need thereof. In one embodiment said use comprises transplantation of all cells obtained from dissociated islet-like cell aggregates or a subset of said, such as monohormonal pancreatic β-cells, into a patient in need thereof.

It is envisioned that said isolated islet-like cell aggregates or cells therefrom will be useful as a pharmaceutical composition. As used herein, the term “pharmaceutical composition” encompasses compositions from cell therapy. Thus, in a related fifth aspect of the present disclosure, there is provided a pharmaceutical composition comprising isolated pancreatic islet-like cell aggregates or cells therefrom as disclosed herein and at least one pharmaceutically acceptable excipient or carrier.

It is also envisioned that it may be beneficial to administer the isolated pancreatic islet-like cell aggregates or cells therefrom as disclosed herein or the pharmaceutical composition as disclosed herein together with a carrier substrate which promotes the growth, proliferation and/or optionally differentiation of said cell population. Thus, in a fourth sixth aspect of the present disclosure, there is provided a kit of parts comprising isolated pancreatic islet-like cell aggregates or cells therefrom as disclosed herein or a pharmaceutical composition as disclosed herein and a suitable carrier substrate. Said suitable carrier substrate may be a 3D scaffold.

In one embodiment there is provided a kit of parts, wherein the carrier substrate is a 3D substrate and wherein said cells are monohormonal β-cells. In other embodiments said kit of parts comprises a 2D substrate. Non-limiting examples of such 2D substrates are the substrates discussed herein.

It will be appreciated that the isolated pancreatic islet(s) or cells therefrom as disclosed herein may have many uses in biological research, for example for drug screening, such as in vitro drug screening. It will be appreciated that the isolated pancreatic islet(s) or cells therefrom as disclosed herein will be beneficial as the percentage of total cells of the desired cell type will be higher than in islet-like cell aggregates of cell populations obtained by culture methods previously described. Hence, the inventive islet-like cell aggregates or cells therefrom will to at least a lower extent compared islet-like cell aggregates or cells generated according to the prior art, if at all, need to be subjected to cell sorting or similar stressful and potentially damaging techniques in order to obtain homogenous populations with high percentage of the desired cell type. Homogenous populations with high percentage of the desired cell type are likely to generate data in drug screening assays which data is less or not obstructed by potential response(s) from other unrelated or contaminating cell types. Thus, in a seventh aspect of the present disclosure, there is provided a use of isolated pancreatic islet-like cell aggregates or cells therefrom as disclosed herein in drug screening, such as in vitro drug screening.

In this context, a method for in vitro drug screening is provided, comprising the steps of generating isolated pancreatic islet-like cell aggregates or cells therefrom according to the method as defined herein; and exposing said islet-like cell aggregates or cells to at least one candidate drug compound. Said method may further comprise a step of evaluating the response of said isolated pancreatic islet-like cell aggregates or cells therefrom to said candidate drug compound.

In one embodiment, there is provided a method of in vitro drug screening, comprising the steps of generating isolated pancreatic islet-like cell aggregates according to the method as defined herein; and exposing said islet-like cell aggregates to at least one candidate drug compound. In one embodiment, there is provided a method of in vitro drug screening, comprising the steps of generating isolated pancreatic islet-like cell aggregates according to the method as defined herein; dissociating the islet-like cell aggregates; and exposing at least a fraction of the dissociated islet cells to at least one candidate drug compound.

In an eight aspect there is provided a method of treatment of a patient in need thereof, comprising administering to said patient a therapeutically effective amount of isolated pancreatic islet-like cell aggregates or cells therefrom as disclosed herein. Furthermore, a method of treatment of diabetes a patient in need thereof, comprising the steps of generating isolated pancreatic islet-like cell aggregates or cells therefrom, according to the method as defined herein; and
administering to said patient a therapeutically effective amount of said isolated pancreatic islet-like cell aggregates or cells therefrom. In one embodiment said isolated pancreatic islet-like cell aggregates or cells therefrom are allogeneic and in another embodiment said isolated pancreatic islet-like cell aggregates or cells therefrom are endogenous to the patient. In one embodiment, said method is for the treatment of diabetes, such as type 1 or type 2 diabetes. Hence, said patient may be suffering from type 1 or type 2 diabetes

Thus, there is provided a method of treatment of diabetes in a patient in need thereof, comprising administering to said patient a therapeutically effective amount of isolated pancreatic islet-like cell aggregates or cells therefrom as disclosed herein. Furthermore, there is provided a method of treatment of a patient in need thereof, comprising the steps of generating isolated pancreatic islet-like cell aggregates or cells therefrom, according to the method as defined herein; and
administering to said patient a therapeutically effective amount of said isolated pancreatic islet-like cell aggregates or cells therefrom. Said method may furthermore comprise isolation of cells, such as cells for generation of IPS cells or of stem cells, from said patient and using said cells for generating cells of the isolated pancreatic islet-like cell aggregates or cells therefrom according to the method as defined herein. The islet-like cell aggregates or cells may be administered by transplantation.

Thus in one embodiment, said administration comprises transplantation of said islet-like cell aggregates or cells into said patient.

In related nineth aspect, there is provided a use of isolated pancreatic islet-like cell aggregates or cells therefrom as described herein for the manufacture of a medicament for the treatment of diabetes in a patient in need thereof. In one embodiment, wherein said manufacture of said medicament comprises generation of isolated pancreatic islet-like cell aggregates or cells therefrom by a method as defined herein. In one embodiment, said isolated pancreatic islet-like cell aggregates or cells therefrom are endogenous to the patient, in other words patient specific islet-like cell aggregates or cells. In one embodiment, said isolated pancreatic islet-like cell aggregates or cells therefrom are allogeneic islet-like cell aggregates or cells.

It will be appreciated that any discussion related to the third and fourth aspects of the present disclosure and embodiments thereof is equally relevant for this eight aspect as well as for the related nineth aspect and will not be repeated here merely for the sake of brevity. In one embodiment there is provided a method of treatment of diabetes in a patient in need thereof as disclosed herein, wherein said patient is suffering from type 1 or type 2 diabetes. In one embodiment, said administration comprises transplantation of said population into said patient.

In yet an additional related aspect is provided a method for the generation pancreatic islet-like cell aggregates in vitro, comprising the steps of:
a−1) providing a cell population of primitive gut tube cells, such as primitive gut tube cells characterized by expression of HNF1β and/or HNF4α;
b−1) culturing said cell population of primitive gut tube cells for no more than approximately 54 hours under conditions permissive of differentiation into posterior foregut cells;
c−1) thereby generating a population of posterior foregut cells, such as posterior foregut cells characterized by the expression of PDX1.
a) providing the cell population of posterior foregut cells generated in step c−1), such as posterior foregut cells characterized by expression of PDX1;
b) culturing said cell population of posterior foregut cells for no more than approximately 78 hours, such as no more than approximately 72 hours, under conditions permissive of differentiation into pancreatic progenitor cells;
c) thereby generating a cell population of pancreatic progenitor cells, such as pancreatic progenitor cells characterized by expression of both PDX1 and NKX6.1;
a+1) providing the cell population of pancreatic progenitor cells generated in step c);
b+1) culturing said cell population of pancreatic progenitor cells under conditions permissive of differentiation into endocrine progenitor cells;
c+1) thereby generating a population of endocrine progenitor cells, such as endocrine progenitor cells characterized by the expression of NEUROD1, such as by expression of NKX6.1 and NEUROD1;
i) providing the population of endocrine progenitor (EP) cells generated in step c+1), such as EP cells characterized by the expression of NEUROD1; such as EP cells characterized by the expression of NKX6.1 and NEUROD1;
ii) providing a single cell suspension of said population of EP cells; iii) allowing said population of EP cells in single cell suspension to form 3D structures;
iv) culturing said population of EP cell in the form of 3D structures in 3D culture conditions permissive of differentiation pancreatic monohormonal β-cells to provide pancreatic islet-like cell aggregates; and
v) thereby generating pancreatic islet-like cell aggregates comprising monohormonal β-cells,
wherein said pancreatic islet-like cell aggregates comprise at least 25% monohormonal β-cells.

Similarly to the first aspect, step v) may in relation the present aspect be reworded as “thereby generating population of pancreatic islet-like cell aggregates comprising monohormonal β-cells, wherein said population comprises pancreatic islet-like cell aggregates which comprise at least 25% monohormonal β-cells.” It will be appreciated that the characterization, in particular in terms percentages of different cells, disclosed in the context of the first aspect is equally relevant here and is not repeated here merely for the sake of brevity.

As used herein, the wording “population of” in reference to a certain type of cells, is meant to be interpreted as a population comprising said cells. For example, the wording “population of EP cells” is to be understood as a population comprising EP cells. The population may in addition also comprise other cells, for example but not limited to cells of earlier developmental stage, such as for example PP cells.

As used herein, when the term “about” or “approximately” is used in relation to a numerical value, it is to be interpreted as a range of ±10%, such as ±9%, such as ±8%, such as ±7%, such as ±6%, such as ±5%, such as ±4%, such as ±3%, such as ±2%, such as ±1%. For example, when the value is stated to be about 10, this means that the value is in fact in the range of from 9 to 11, such as in the range of from 9.9 to 10.9, such as in the range of from 9.8 to 10.8, such as in the range of from 9.7 to 10.7, such as in the range of from 9.6 to 10.6, such as in the range of from 9.5 to 10.5, such as in the range of from 9.4 to 10.4, such as in the range of from 9.3 to 10.3, such as in the range of from 9.2 to 10.2, such as in the range of from 9.1 to 10.1.

The skilled person knows that numerical values relating to measurements are subject to measurement errors which place limits on their accuracy. For this reason, the general convention in the scientific and technical literature is applied: the last decimal place of a numerical value indicates its degree of accuracy. Where no other error margins are given, the maximum margin is ascertained by applying the rounding-off convention to the last decimal place e.g. for a measurement of 3.5 cm, the error margin is 3.45-3.54. When interpreting ranges of values in patent specifications, the skilled person proceeds on the same basis.

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation, culture conditions or cell population to the teachings of the invention without departing from the essential scope thereof. Therefore, it is intended that the invention not be limited to any particular embodiment contemplated, but that the invention will include all embodiments falling within the scope of the appended claims.

### BRIEF DESCRIPTION OF THE FIGURES

FIGS. 1A-1B are a schematic illustration of the developmental stages along the pancreatic β-cell lineage including the expression of markers characteristic of each developmental stage. (FIG. 1A) Pancreatic endocrine cell types can be generated from human pluripotent embryonic stem cells (hES) or IPS cells by recapitulating embryonic pancreas development in the petri dish. The pancreatic differentiation can be divided into multiple stages including definitive endoderm (DE), primitive gut tube (PGT), posterior foregut (PF), multi-potent pancreatic progenitor (PP), endocrine progenitor (EP), and pancreatic islet (ISL). The key markers expressed at each stage are shown in the figure. (FIG. 1B) FIG. 1B is a schematic illustration of the long differentiation protocol according to the prior art and the short differentiation protocol as disclosed herein. The effects of long and short durations for stage 3 and 4 (S3+S4) on subsequent endocrine differentiation were analyzed. The expression of stage-specific markers was examined at the end of stage 2 (S2), stage 3 (S3), stage 4 (S4), four days into stage 5 (S5), and stage 6 (S6). 2D LN-521 is an example of a 2D substrate and may be replaced by other 2D substrates as disclosed herein.

FIGS. 2A-2C show a comparison of pancreatic differentiation on different coating substrates. HS980 and H1 cells were differentiated on Matrigel, LN-511, and LN-521 coated plates using the long protocol. The expression of key progenitor markers PDX1, NKX6.1 and NEUROD1 were examined by flow cytometry at the end of S4 or 4 days into S5. Representative dot plots for HS980 cells at (FIG. 2A) stage 4 and (FIG. 2B and FIG. 2C) 5 are shown. Bar graphs representing the results for both HS980 and H1 cells are shown. The results were calculated from three independent samples. To compare the percentages of (FIG. 2A and FIG. 2B) PDX1+/NKX6.1⁺ and (FIG. 2C) NKX6.1⁺/NEUROD1⁺ cells among the three substrates, unpaired 2-tailed t-tests were performed in Microsoft Excel. *p<0.05, **p<0.01, ***p<0.001, ****p<0.0001, *****p<0.00001.

FIGS. 3A-3B show a comparison of different lengths of culture of primitive gut tube (PGT) cells to posterior foregut cells (PF). HS980 and H1 cells were differentiated towards S3 on LN-521 coated plates. The expression of PF marker PDX1 were analyzed by immunocytochemistry (ICC) and flow cytometry at the end of S2, or 1 or 2 days into S3. Representative confocal microscope pictures and dot plots for HS980 cells are shown. (FIG. 3A) HS980 cells. (FIG. 3B) H1 cells.

FIGS. 4A-4G show results from the evaluation of the effects of S4 duration on endocrine differentiation. HS980, H1 and H9 cells were differentiated on LN-521 coated plates. The durations of S4 were 1 day, 2 days, 3 days, 4 days, or 5 days. The expression of progenitor makers NKX6.1 and NEUROD1 were examined by flow cytometry at the end of S4 or four days into S5. The expression of endocrine marker INSULIN (INS) and GLUCAGON(GCG) were examined at the end of S6. The results were calculated from multiple independent experiments. To compare the percentages of different cell populations among the different S4 durations, unpaired 2-tailed t-tests were performed in Microsoft Excel. (FIG. 4A) Schematic illustration of different durations for S4. (FIG. 4B) Representative dot plots from analysis of expression of progenitor makers NKX6.1 and NEUROD1 in HS980 cells at S4 and S5. (FIG. 4C) Representative dot plots from analysis of expression of progenitor makers NKX6.1 and NEUROD1 in H1 and H9 cells at S5. (FIG. 4D) Bar graphs showing calculated percentage of NKX6.1⁺/NEUROD1⁺ and NKX6.1⁺/NEUROD1-cells. Results show percentages of H980, H1 and H9 cells. (FIG. 4E) Bar graphs showing calculated percentage of NEUROD1⁺ and NKX6.1⁺ cells at S5. Results show percentages of H980, H1 and H9 cells. (FIG. 4F) Representative dot plots from analysis of expression of INS and GCC in HS980, H1 and H9 cells at S6. (FIG. 4G) Bar graphs showing calculated percentage of total INS⁺/GCG-mono-hormonal β-cells and GCG+/INS-mono-hormonal α-cells. *p<0.05, **p<0.01, ***p<0.001, ****p<0.0001, *****p<0.00001.

FIGS. 5A-5C show the evaluation of pancreatic differentiation on different coating substrates. HS980, H1, and H9 cells were differentiated towards S5 EP cells on plates coated with different recombinant human Laminin isoforms (LN), or Matrigel using the short protocol. H1 cells were also differentiated on plate coated with Fibronectin (FN) or Vitronectin (VTN). Four days into S5 the expression of NKX6.1 and NEUROD1 were measured by flow cytometry. The HS980 cells on LN-332, LN-511, LN-521, and Matrigel were further differentiated towards S6 islet cells in 3D suspension. The expression of INS and GCG were measured by flow cytometry at the end of S6. Representative dot plots are shown in FIGS. 5A-1 and 5A-2 for S5 EP cells, and in FIG. 5C for S6 islet cells. To compare the percentage of NKX6.1⁺/NEUROD1⁺ cells between laminin isoforms and Matrigel, paired 2-tailed t-tests were performed in Microsoft Excel. (FIG. 5B) Percentage of NKX6.1⁺/NEUROD1⁺ cells. *p<0.05, **p<0.01.

FIGS. 6A-6B illustrate the effect 3D suspension of S4 and S5 progenitor cells culture on islet formation. (FIG. 6A) Schematic representation showing the timeline of when HS980 cells were differentiated on LN-521 using the short protocol. The cells were dissociated into single cells at the end of S4 (upper timeline), or 4 or 6 days into S5 (lower timeline). 4×10⁶ S4 and S5 cells per well were maintained in 3D suspension and further differentiated into S6 islet cells for analysis. (FIG. 6B) At the end of S6, the islet-like aggregates were counted under microscope and then dissociated into single cells for cell number counting. The expression of INS and GCG was measured by flow cytometry. Representative dot plots are shown. The percentages of cell populations and the numbers of aggregates/cells per well are shown in the bar graphs and were calculated from three independent samples. Unpaired 2-tailed t-tests were performed in Microsoft Excel. *p<0.05, **p<0.01, ***p<0.001, ****p<0.0001, *****p<0.00001.

FIGS. 7A-7D show the enrichment of S5 EP cells during aggregate formation in suspension. HS980 and H1 cells were differentiated towards S5 on LN-521 coated 2D surface. The cells were dissociated into single cells 4 days into S5 and then maintained in suspension for one day to generate 3D aggregates. The expression of NKX6.1, NEUROD1, and Ki-67 were examined by flow cytometry before and after aggregate formation. To investigate the effects of ROCK inhibitor H1152 on aggregate formation, different concentrations of H1152, from 0 to 10 μM, were added to single cell suspension as shown. Next day, the expression of markers and the islet cell numbers were measured. Representative dot plots were shown in (FIG. 7A), and (FIG. 7B) and (FIG. 7C). The percentages of NKX6.1⁺/NEUROD1⁺ and NEUROD1⁺ cells were calculated from three independent experiments and as shown as bar graphs in (FIG. 7A). The islet cell numbers generated from 10⁶ single cells in 3D suspension were calculated from three independent samples and as shown as bar graphs in (FIG. 7D). Unpaired 2-tailed t-tests were performed in Microsoft Excel. *p<0.05, **p<0.01, ***p<0.001, ****p <0.0001, *****p<0.00001.

FIGS. 8A-8D show the results from a comparison of short and long pancreatic differentiation protocols. HS980 and H1 cells were differentiated into S5 EP cells on LN-521 coated plates using the short protocol as disclosed herein and the comparative long protocol. Four days into S5 the cells were dissociated into single cells and further maturated into islet-like cell aggregates in 3D suspension. The expression of key markers was examined by flow cytometry at each stage as shown (S3-S6). In vitro glucose-stimulated insulin c-peptide secretion was examined at the end of S6. The islet-like aggregates were counted under microscope and then dissociated into single cells for cell number counting. The results were calculated from multiple independent samples. Unpaired 2-tailed t-tests were performed in Microsoft Excel. (FIG. 8A) Representative dot plots of the expression of NKX6.1 and NEUROD1 in HS980 cells during S3-5. (FIG. 8B) Bar graphs showing quantification of NKX6.1⁺/NEUROD1⁺ and NKX6.1⁺/NEUROD1-obtained using short and long protocols. (FIG. 8C) Representative dot plots of expression of INS and GCG in HS980 and H1 cells at S6 obtained using the short and long protocols and bar graphs showing quantification thereof. (FIG. 8D) Bar graphs showing comparison of results of in vitro glucose stimulated insulin secretion in cells obtained using short and long protocols and bar graphs showing the numbers of islet-like aggregates and islet cells per well in cultures using short and long protocols. HS980 cell aggregates were analyzed at S6. *p<0.05, **p<0.01, ***p<0.001, ****p<0.0001, *****p<0.00001.

FIGS. 9A-9C show immunocytochemistry analysis of expression of stage specific markers in cell cultures. HS980 cells differentiated using the short and long protocols were analyzed using antibodies specific against key markers expressed during S4-S6. (FIG. 9A) Expression of PDX1, NKX6.1, and NEUROD1 in S4 cells. (FIG. 9B) Expression of PDX1, NKX6.1, and NEUROD1 in S5 cells. (FIG. 9C) Expression of INSULIN c-peptide (CPEP), GLUCAGON(GCG), and SOMATOSTATIN (SST) in S6 cells. DAPI staining was used to visualize cells.

FIGS. 10A-C show the results from a comparison between spontaneous aggregation in 3D suspension and forced aggregation using microwell plates. H1 cells were differentiated towards S5 on LN-521 coated 2D surface. The cells were dissociated into single cells four days into S5 and then maintained in 3D suspension (free), 96-well plates (96-well), or AggreWell plates (AggreWell). The expression of NKX6.1, NEUROD1 and Ki-67 were examined by flow cytometry before and one day after aggregate formation at S5. Representative dot plots were shown in (FIG. 10A). The percentages of S5 NKX6.1⁺/NEUROD1⁺ EP cells and Ki-67⁺ proliferative cells were calculated from multiple independent samples and as shown as bar graphs in (FIG. 10B). Differentiation towards S6 islet cells were carried out in suspension and AggreWell plates. The expression of INS, GCG, and Ki-67 were examined by flow cytometry at the end of S6. Representative dot plots were shown in (FIG. 10C). Unpaired 2-tailed t-tests were performed in Microsoft Excel. *p<0.05, **p<0.01.

FIG. 11 is a schematic illustration of the in vitro differential protocol as disclosed herein. Factors and durations for each stage were as shown. The cells on LN-521 coated surface were dissociated into single cells at day 14 and then maintained in 3D suspension.

FIGS. 12A-12B show pancreatic differentiation in human ESC and iPSC lines. The cells were differentiated using the short protocol. The expression of key markers was examined by flow cytometry four days into S5 and at the end of S6. In vitro glucose-stimulated insulin c-peptide secretion was examined at the end of S6. The results were calculated from multiple independent experiments. Unpaired 2-tailed t-tests were performed in Microsoft Excel. *p<0.05, **p<0.01, ***p<0.001, ****p<0.0001, *****p<0.00001. Representative dot plots of INS and GCG expression at S6 and NKX6.1 and NEUROD1 expression at S5 were shown in (FIG. 12A) and (FIG. 12B). Bar graphs showing quantification of INS⁺/GCG−, INS−/GCG⁺, and INS⁺/GCG⁺ cell populations (left) and bar graphs showing results of in vitro glucose stimulated insulin secretion (right) were shown in (FIG. 12A). (FIG. 12A) Differentiation of hESC lines. (FIG. 12B) Differentiation of human iPSC line C7.

FIGS. 13A-13B show the results from a comparison between the short protocol and two published protocols by scRNA sequencing. H1 cells were differentiated using the short protocol and scRNAseq was performed at the end of S6. (FIG. 13A) Illustration of the short protocol and two published protocols. Durations, factors, and 2D/3D culture systems for each stage were shown. (FIG. 13B) UMAP with cell type prediction (upper) and expression level of marker genes INS and GCG in the predicted cell types (under).

FIG. 14 shows that frozen S5 EP cells were able to generate S6 islet-like aggregates. HS980 and H1 cells were differentiated towards S5 on LN-521 coated 2D surface. The cells were dissociated into single cells four days into S5 and then maintained in 3D suspension to generate S6 aggregates. Alternatively, the single cells were frozen and kept in liquid nitrogen. To generate 3D aggregates, the frozen S5 cells were thawed and cultured in 3D suspension. The expression of markers INS and GCG and in vitro glucose stimulated insulin c-peptide release were examined at the end of S6. Bar graphs showing quantification of INS⁺/GCG−, INS−/GCG⁺, and INS⁺/GCG⁺ cell populations (left) and bar graphs showing results of in vitro glucose stimulated insulin secretion (right) were shown. The results were calculated from multiple independent experiments. Unpaired 2-tailed t-tests were performed in Microsoft Excel. *p<0.05, **p<0.01.

### EXAMPLES

Summary: It the present examples it is demonstrated that the inventive method disclosed herein, comprising a step of culturing a cell population of posterior foregut cells under conditions permissive of differentiation into pancreatic progenitor cells for no more than approximately 78 hours, such as for 72 hours or 48 hours, leads to increase numbers of endocrine precursor cells, which have the ability to develop into mature pancreatic β-cells.

### Materials and Methods

hESC Culture

hESC line HS980 was derived under xeno-free and defined conditions as previously described (Rodin, S., et al 2014). hESC lines WA01/H1 and WA09/H9 were obtained from WiCell Research Institute (Madison, Wisconsin). Human iPSC line CTRL-7-II (C7) is described in Kele M et al., 2016). The hESC lines were maintained in NutriStem hPSC XF Medium (Biological Industries, Israel; 05-100-1A) on Sarstedt multi-well cell culture plates coated with 10 μg/mL human recombinant Laminin (LN)-521 (BioLamina, Sweden; LN-521), in a 37° C. incubator with 5% CO₂, 5% O₂ and 100% humidity. The hESCs were enzymatically passaged at 12000-24000 cells per cm² every 3-5 days.

For routine passaging, hESC cultures on LN-521 were briefly washed in D-PBS without Ca²⁺ and Mg²⁺ (Thermo Fisher; 14190169) and incubated with Gibco TrypLE Select (Thermo Fisher; A1285901) for 5 minutes at 37° C. The cells were collected into fresh NutriStem hPSC XF medium by pipetting gently 5-10 times using a P1000 pipette, centrifuged at 300 g for 4-5 minutes, resuspended into fresh NutriStem hPSC XF medium, and seeded onto freshly coated cell culture plates.

In Vitro Pancreatic Differentiation of hESCs

The stepwise pancreatic differentiation protocols described here were modified from previously published protocols (Pagliuca et al., 2014; Rezania et al., 2014; Millman et al., 2016; Vegas et al., 2016).

The hESC lines H1, H9 and HS980 were seeded onto LN-521 coated cell culture plates at 24000 cells per cm² in NutriStem hPSC XF medium. The pancreatic differentiation was initiated four days later, resulting in 95-100% confluency. The differentiating cell cultures were maintained in a 37° C. incubator with 5% CO₂, 20% O₂ and 100% humidity.

The differentiation can be divided into six stages, from S1 to S6, and media used for each stage were as follows:
S1 media: MCDB131 (Thermo Fisher; 10372019)+25 mM NaHCO₃(Sigma; S6297)+1×GlutaMAX (Thermo Fisher; 35050038)+50 U/mL Penicillin-Streptomycin (Thermo Fisher; 15140122)+2.5 mM D-Glucose (8 mM final concentration, Sigma; G8769)+0.2% or 0.5% Fatty Acid Free Bovine Serum Albumin (FAF-BSA, Sigma; A8806).
S2 media: MCDB131+25 mM NaHCO₃+1X GlutaMAX+50 U/mL Penicillin-Streptomycin+2.5 mM D-Glucose (8 mM final concentration)+0.2% or 0.5% FAF-BSA+0.25 mM Vitamin C (Sigma; A4544).
S3 media: MCDB131+25 mM NaHCO₃+1X GlutaMAX+50 U/mL Penicillin-Streptomycin+2.5 mM D-Glucose (8 mM final concentration)+0.5% FAF-BSA+0.25 mM Vitamin C+1:200 ITS-X (Thermo Fisher; 51500056).
S5 media: MCDB131+25 mM NaHCO₃+1X GlutaMAX+50 U/mL Penicillin-Streptomycin+14.5 mM D-Glucose (20 mM final concentration)+0.5% FAF-BSA+1:200 ITS-X+10 μM ZnSO₄ (Sigma; Z0251)+10 μg/mL Heparin (Sigma; H3149).
S6 media: CMRL (Thermo Fisher; 11530037)+14 mM NaHCO₃+1× GlutaMAX+50 U/mL Penicillin-Streptomycin+14.5 mM D-Glucose (20 mM final concentration)+1% FAF-BSA+1:200 ITS-X (for three weeks)+10 μM ZnSO₄+10 μg/mL Heparin+1X NEAA (Thermo Fisher; 11140035).

### The Short Differentiation Protocol

Stage 1 definitive endoderm (3 days): Undifferentiated H1, H9, and HS980 cells were rinsed once with D-PBS with Ca²⁺ and Mg²⁺ (Thermo Fisher; 14040091) and then induced with 5 μM CHIR99021 (Tocris; 4423) and 100 ng/ml Activin A (R&D; 338-AC) for 24 hours in S1 media. For the next two days the cells were fed every day with S1 media containing only 100 ng/ml Activin A. The concentrations of FAF-BSA in S1 media were 0.2% for HS980 cells and 0.5% for H1 and H9 cells.

Stage 2 primitive gut tube (3 days): The cells were induced with 50 ng/ml KGF (R&D; 251-KG) in S2 media for three days. The concentrations of FAF-BSA were 0.2% for HS980 cells and 0.5% for H1 and H9 cells.

Stage 3 posterior foregut (1 day): The cells were induced with 50 ng/ml KGF, 2 μM Retinoic acid (Sigma; R2625), 0.25 μM SANT-1 (Sigma; S4572), 0.5 μM PDBu (Tocris; 4153), and 200 nM LDN193189 (Tocris; 6053) in S3 media for 24 hours.

Stage 4 pancreatic progenitor (2 or 3 days): The cells were induced with 50 ng/ml KGF, 100 ng/ml EGF (R&D; 236-EG), 5 ng/ml Activin A, 10 mM Nicotinamide (Sigma; N0636), 100 nM Retinoic acid, 0.25 μM SANT-1, 0.5 μM PDBu, and 200 nM LDN193189 in S3 media for 3 days. To analyze the duration effects of stage 4, the cells were also differentiated for 1-5 days during this step.

Stage 5 endocrine progenitor (5 days): The cells were induced with 20 ng/ml Betacellulin (R&D; 261-CE), 100 nM Retinoic acid, 0.25 μM SANT-1, 100 nM GSI-XX (Sigma; 565789), 10 μM ALK5 inhibitor II (Cayman Chemical; 14794), 1 μM GC-1 (Tocris; 4554), and 100 nM LDN193189 in S5 media.

Four days into stage 5, the cells were rinsed once with DPBS without Ca²⁺ and Mg²⁺, treated with Accutase for 10 minutes at 37° C., and then dissociated into single cells in S5 media by pipetting multiple times using a P1000 pipette. The single cells were pelleted by centrifugation at 300 g for 5 minutes, and then resuspended at 1.0-1.5×10⁶ cells/ml in S5 media supplemented with 10 μM H1152 (Tocris; 2414) and the other factors. To generate islet-like aggregates, the cells were transferred to ultra-low attachment 6-well plates (Corning; 3471), totally 4-6×10⁴ cells in 4 ml per well, and incubated overnight on an orbital shaker (Infors HT Celltron) at 95 rpm in the incubator.

To investigate the effects of ROCK inhibitor H1152 on cell survival and aggregate formation, different concentrations of H1152, from 0 to 10 μM, were added to single cell suspension for one day.

Stage 6 (4 weeks): The cell aggregates were maintained in S6 media further supplemented with 10 μM H1152, 1 PM GC-1, 10 μM Trolox (Merck Millipore; 648471) and 1 mM N-acetyl-L-cysteine (Sigma; A9165). ITS-X and H1152 were removed from the media after three weeks. The aggregates were kept on orbital shaker at 95 rpm in the incubator.

The medium was changed every day from stage 1 to 5, and every 2-3 days during stage 6.

### The Long Differentiation Protocol:

hESCs were differentiated towards endocrine progenitors using the same factors and media as the short protocol described above, but the durations for stage 3 and 4 were 2 and 5 days, respectively.

### Freezing of Stage 5 Endocrine Progenitor Cells

Four days into S5, the cells were dissociated into single cells as described above. The single cells were pelleted by centrifugation at 300 g for 5 minutes, and then resuspended at 1×10⁷ cells/ml in cold STEM-CELLBANKER GMP grade solution (amsbio, 11924). The cell suspension was dispersed into Nunc cryogenic tubes (Thermo Fisher; 377267), totally 1-1.5 ml per tube, and cooled to −80° C. using a Mr. Frosty freezing container (Thermo Fisher; 5100-0001). For controlled cooling, a programmable cooling unit can be used to cool the cells at 1° C. per minute. For long-term cryoperservation, the cryogenic tubes were transferred to liquid nitrogen storage.

### Thawing of Stage 5 Endocrine Progenitor Cells

The frozen S5 cells in cryogenic tube were removed from the liquid nitrogen storage and rapidly thawed at 37° C. Each 1 ml cell suspension was diluted and gently mixed in 5 ml pre-warmed complete S5 media. The cells were pelleted by centrifugation at 300 g for 5 minutes, and then resuspended at 1.0-1.5×10⁶ cells/ml in complete S5 media. Further differentiation prodecures were carried out as described above.

### Aggregate Formation in Microwells

To generate islet-like aggregates in microwells, the S5 cells were dissociated into single cells as described above. The single cells were resuspended at 1.5 ×10⁶ cells/ml and then seeded to AggreWell 400 6-well plate (Stem Cell Technologies; 34421), totally 6×10⁴ cells in 4 ml per well. Alternatively, 10⁴ single cells in 50 μl were seeded to each well on 96-well Microtest plates (Sarstedt, 82.1583.001). The cells were then incubated overnight in the incubator.

Differentiation into S6 pancreatic islet-like cell aggregates was carried out as described above. The aggregates were kept in AggreWell 400 6-well plates without shaking.

### Aggregate and Cell Counting

The S6 islet-like aggregates were counted under brightfield microscope. The aggregates were then rinsed once in D-PBS without Ca²⁺ and Mg²⁺ and incubated with Accutase for 15 minutes at 37° C. The aggregates were dissociated into single cells by pipetting multiple times using a P1000 pipette, centrifuged at 300 g for 4-5 minutes, and resuspended into D-PBS without Ca²⁺ and Mg²⁺. The cell number was counted using ORFLO MOXI Z Mini Automated Cell Counter (ORFLO; MXZ001).

### Flow Cytometry

Cells were dissociated into single cells by treatment with Accutase for 15 min at 37° C. Then the cells were washed twice and resuspended at 10⁶ cells/ml in D-PBS without Mg²⁺ and Ca²⁺. The cells were incubated for 30 minutes on ice with LIVE/DEAD fixable dead cell stain kit (Thermo Fisher; L34963 and L34965). After washing twice with D-PBS without Mg²⁺ and Ca²⁺, the cells were fixed with BD Bioscience Cytofix/Cytoperm buffer (554722) for 20 minutes on ice. The cells were then washed twice in 1X BD Bioscience Perm/Wash buffer (554723) and incubated with conjugated antibodies diluted in 1X BD Bioscience Perm/Wash buffer for 30 minutes on ice. After washing twice in 1X BD Bioscience Perm/Wash buffer, the cells were resuspended in FACS buffer (D-PBS without Ca2+ or Mg2+ containing 2% fetal bovine serum (Thermo Fisher; 10082147) and 1 mM EDTA (Thermo Fisher; 15575020)) and analyzed using a Beckman Coulter CytoFLEX S flow cytometer. The flow cytometry data were analyzed using BD Bioscience FlowJo v10.8 software. The conjugated antibodies were all purchased from BD Bioscience: Alexa Fluor 647 mouse anti-Insulin (1/20, 565689), PE mouse anti-Glucagon (1/20, 565860), Alexa Fluor 488 mouse anti-human Somatostatin (1/20, 566032), PE mouse anti-NEUROD1 (1/20, 563001), Alexa Fluor 647 mouse anti-NKX6.1 (1/20, 563338), Alexa Fluor 488 mouse anti-PDX-1 (1/20, 562274), and V450 mouse anti-Ki-67 (1/20, 561281).

### Immunofluorescence

Cells were fixed with 4% (wt/vol) paraformaldehyde for 20 min at room temperature (RT) and subsequently washed with D-PBS without Mg2+ and Ca²⁺ for three times. For immunostaining, cells were blocked with 5% normal donkey serum (Merck Millipore; S30-100 mL) in D-PBS without Mg2+ and Ca²⁺ containing 0.3% (vol/vol) Triton X-100 (Sigma; T9284) for 1 hour at RT, and then incubated with primary antibodies diluted in D-PBS without Mg2+ and Ca²⁺ containing 0.1% (vol/vol) Triton X-100 or Tween 20 (Sigma; P9416) and 5% normal donkey serum overnight at 4° C. Next day, the cells were incubated with secondary antibodies for 1 hour at RT. After each incubation step the cells were washed with D-PBS without Mg2+ and Ca²⁺ for three times. The primary antibodies used were as follows: goat anti-human PDX-1 (1/300, R&D; AF2419), mouse anti-Nkx6.1 (1/100, DSHB; F55A12-s), sheep anti-human Neurogenin-3 (NGN3) (1/100, R&D; AF3444), goat anti-human/mouse NeuroD1 (1/100, R&D; AF2746), guinea pig anti-C-Peptide (1/100, abcam; ab30477), rat anti-C-Peptide (1/50, DSHB; GN-ID4-s), mouse anti-Glucagon (1/1000, Sigma; G2654), and rabbit anti-Somatostatin (1/500, Sigma; 332A-1).

### In Vitro Glucose Stimulation

Twenty to thirty hESC-derived aggregates at the end of stage 6 were incubated overnight in S6 media without ITS-X and additional Glucose (5 mM final). Next day the aggregates were transferred to a 24-well ultra-low attachment plate (Corning; 3473) and washed twice with 2 ml Krebs buffer (129 mM NaCl, 4.8 mM KCl, 2.5 mM CaCl₂), 1.2 mM MgSO₂, 1 mM Na₂HPO₄, 1.2 mM KH₂PO₄, 5 mM NaHCO₃, 10 mM HEPES, and 0.1% FAF-BSA). The aggregates were then pre-incubated in 2 ml Krebs buffer containing 2 mM Glucose for 2 hours to remove residual insulin. After that, the aggregates were washed twice with 2 ml Krebs buffer and then incubated in 2 ml Krebs buffer containing 2 mM Glucose for 30 min. A sample of 500 μl supernatant was collected after incubation (low glucose sample). The aggregates were washed once with 2 ml Krebs buffer and then incubated in 2 ml Krebs buffer containing 20 mM Glucose for 30 min. A sample of 500 μl supernatant was collected after incubation (high glucose sample). The aggregates were washed twice in 2 mL Krebs buffer and then incubated again in 2 mL Krebs buffer containing 2 mM glucose for 30 min. A sample of 500 μL of the supernatant was collected (low glucose sample). The aggregates were washed once in 2 mL Krebs buffer and then incubated in 2 mL Krebs buffer containing 2 mM glucose and 30 mM KCl (polarization challenge) for 30 min. A sample of 500 μl of the supernatant was collected (KCl challenge sample). After the KCl challenge, the aggregates were dispersed into single cells by treatment with Accutase for 15 minutes and the total cell numbers were counted using an ORFLO MOXI Z cell counter. The collected supernatant samples containing secreted insulin were processed using human c-peptide ELISA kit (R&D; DICP00). Human insulin c-peptide measurements were normalized by the total cell numbers and presented as pmol c-peptide released from 10³ cells. If the ELISA was not performed on the same day, samples were stored at −80° C.

scRNA Sequencing Sample Preparation

hESC-derived islets were collected for scRNA sequencing at the end of S6. The islet-like aggregates were rinsed twice in D-PBS without Ca²⁺ and Mg2+ and incubated with TrypLE for 15-20 minutes on orbit shaker at 37° C. The aggregates were dissociated into single cells by pipetting multiple times using a P1000 pipette, centrifuged at 300 g for 4-5 minutes, resuspended into D-PBS without Ca²⁺ and Mg²⁺ containing 0.04% FAF-BSA, and then filtrated using a 40 μm cell strainer (VWR, 732-2760). To determine total cell number and live cell ratio, single cell suspension was stained with 0.4% Trypan blue solution (ThermoFisher, 15250061) and counted using a hemocytometer.

### Single-Cell RNA Sequencing Analysis

3000 cells were used for construction of scRNA sequencing libraries. The 10× Genomics Chromium Next GEM Single Cell 3′ Reagent Kits v3.1 (10x genomics, CG000315 or CG000388) was used, sometimes together with Cell Multiplexing Oligo Labeling protocol (10× Genomics, CG000391), followed by RNA sequencing on Illumina Nextseq 2000 machine. To generate FastQ file and feature-barcode matrices, analysis steps were carried out in Cell Ranger 3.1.0. Uniform Manifold Approximation and Projection (UMAP) for dimension reduction, cell type identification, and differential gene expression analysis are performed.

### Example 1

In this example a comparison of cell culture substrates was made for their ability to support pancreatic differentiation using to different hESC lines. Pancreatic differentiation was evaluated based on expression key markers for pancreatic progenitor (PP) cells at stage 4 (S4) and endocrrne progenitor (EP) cells at stage 5 (S5).

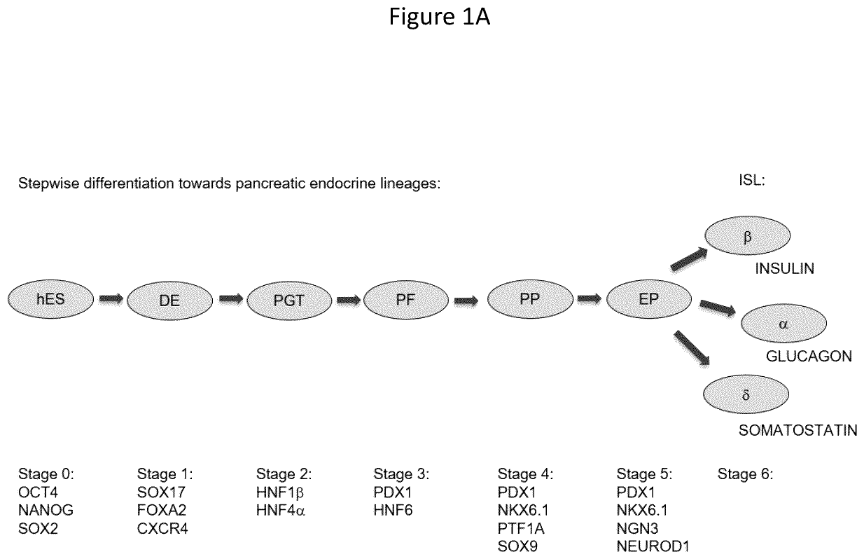

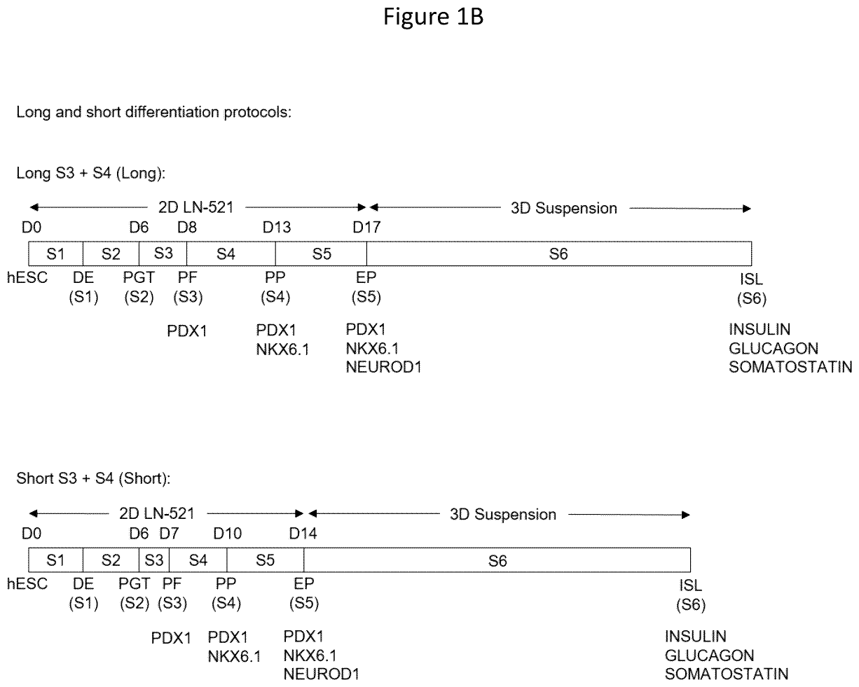

Protocols have been developed for in vitro pancreatic differentiation of hPSCs cultured on 2D surface coated with feeder cells or Matrigel (Pagliuca et a. 2014; D'Amour et al., 2006; Kroon et al., 2008). However, for clinical applications of hPSC-derived pancreatic islets, xeno-free chemically defined coating substrates are highly preferred. We therefore compared human recombinant LN-511 and LN-521 with Matrigel for their ability to support pancreatic differentiation. hESC lines HS980 and H1 were differentiated on these three substrates using the long differentiation protocol (see upper panel FIG. 1B) and the expression of key progenitor markers PDX1, NKX6.1, and NEUROD1 were measured by flow cytometry at the end of S4 and then four days into S5.

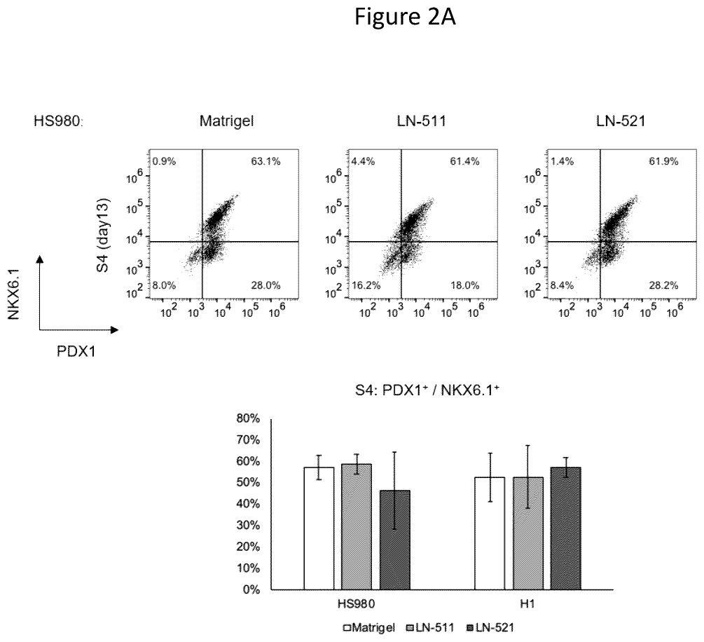

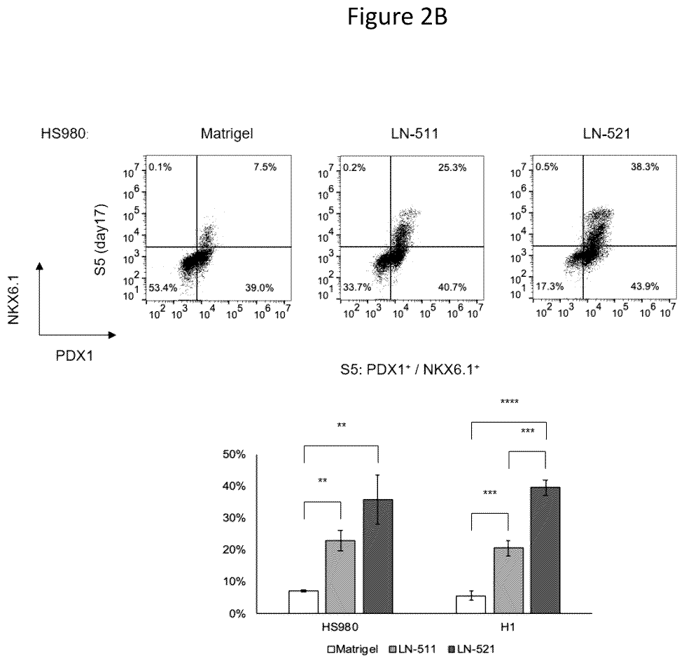

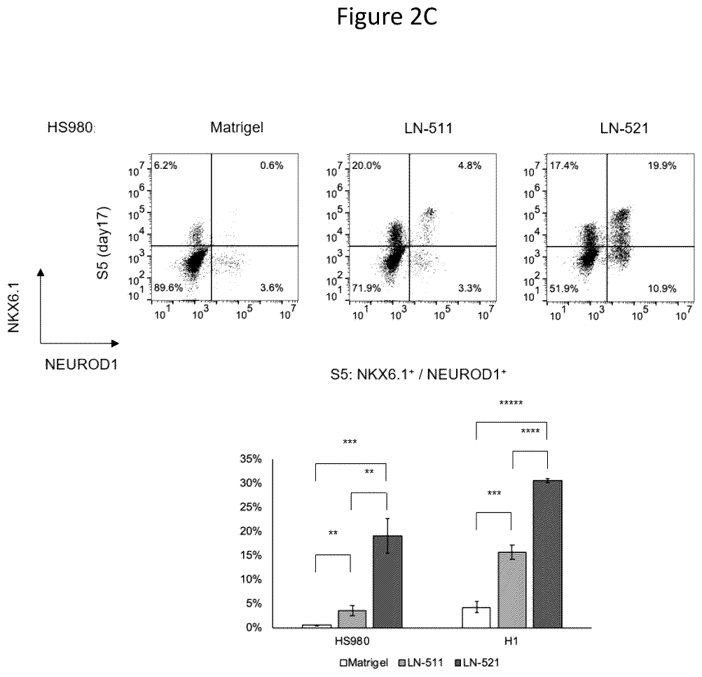

Results: The results showed that the percentages of S4 PDX1+/NKX6.1+ pancreatic progenitor (PP) cells were comparable on all substrates in both HS980 and H1 cell lines (FIG. 2A). However, the level of PDX1+/NKX6.1⁺ cells decreased dramatically on Matrigel and to a lesser extent also on LN-511, but remained relatively stable on LN-521 when the S4 cells were further differentiated towards S5 (FIG. 2B). The percentages of S5 NKX6.1⁺/NEUROD1⁺ endocrine progenitor (EP) cells were much higher on LN-521, and to a lesser extent also on LN-511, than on Matrigel (FIG. 2C).

Taken together, the data showed that Matrigel, LN-511, and LN-521 were able to support differentiation into S4 PP cells, and LN-521 especially also provided a permissive surface environment for endocrine differentiation of S4 PP cells. We therefore decided to continue all our experiments on LN-521 instead of Matrigel. Since the percentages of S5 EP cells were only 20-30% on LN-521, modifications were required in order to optimize the differentiation protocol.

### Example 2

In this example the effects of stage 4 duration on endocrine differentiation were studied.

### Evaluation of Different Lengths of Stage 3

It has previously been reported that a short duration of S3 promotes S4 PDX1⁺/NKX6.1⁺ PP cell populations and inhibits immature poly-hormonal cell populations (Nostro et al., 2015). However, it was unclear if the duration of S4 could also influence later differentiation stages in similar ways. Therefore, we decided to analyze/optimize the durations for both S3 and S4. To identify the minimal duration for S3, HS980 and H1 cells were differentiated towards S3 posterior foregut (PF) cells and the expression of key PF marker PDX1 was measured at the end of S2 and then 1 or 2 days into S3.

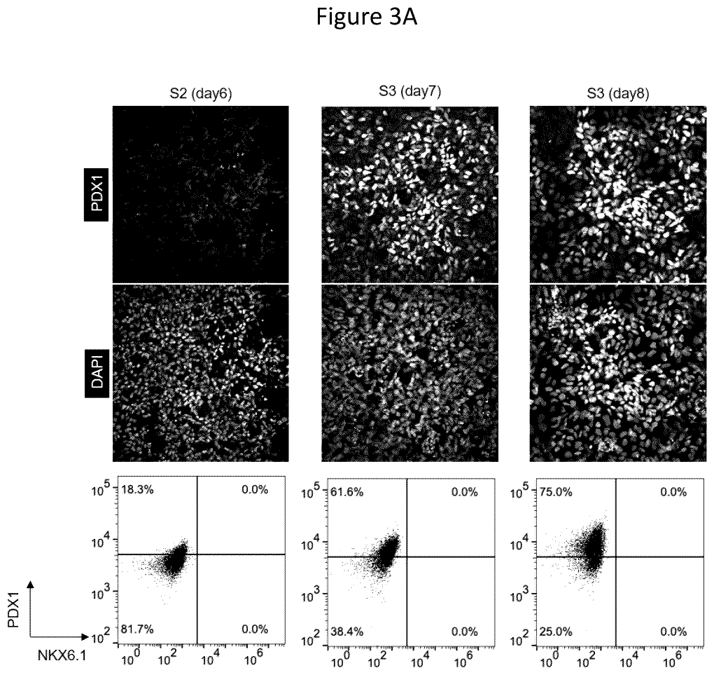

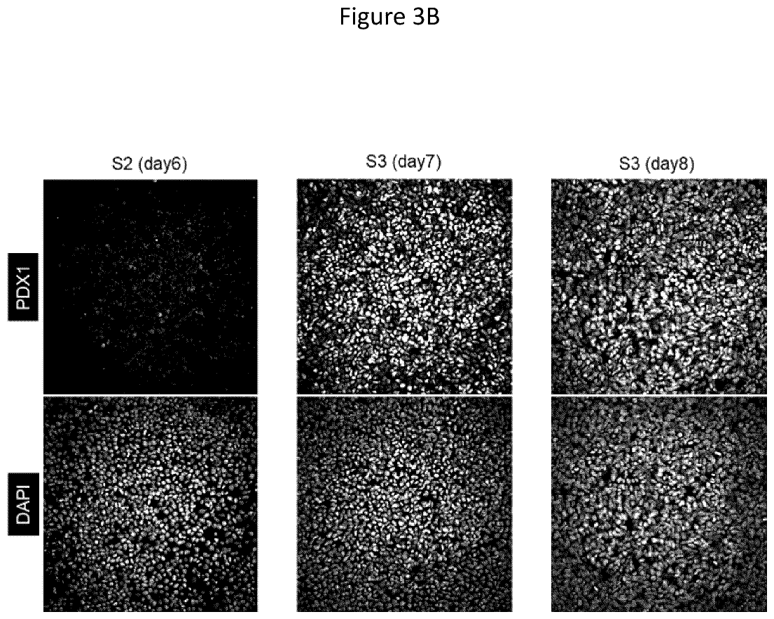

Results: The results showed that PDX1 was already expressed in most of the cells after 1 day into S3, and the expression only slightly increased after 2 day as shown by the immunocytology analysis and the representative dot blots showing the number of PDX1+ cells at day 6, 7 and 8 of culture (FIG. 3). Thus, the data shows that a duration of one day for S3 was sufficient for differentiation of S2 PGT cells into S3 PDX1+PF cells. A duration of two days also resulted in high numbers of S3 PDX1+PF cells. Thus, it was concluded that the duration of S3 should be at most 48 hours, such as for example 24 hours.

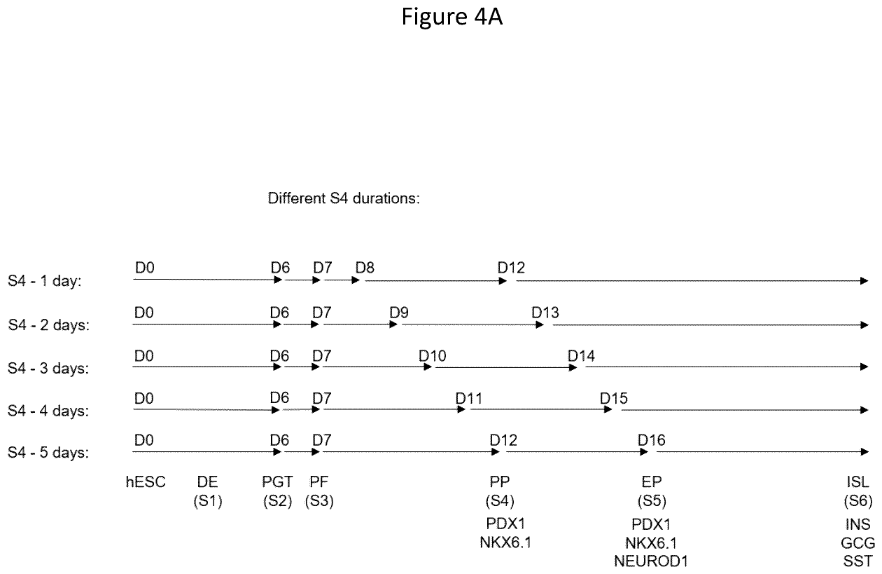

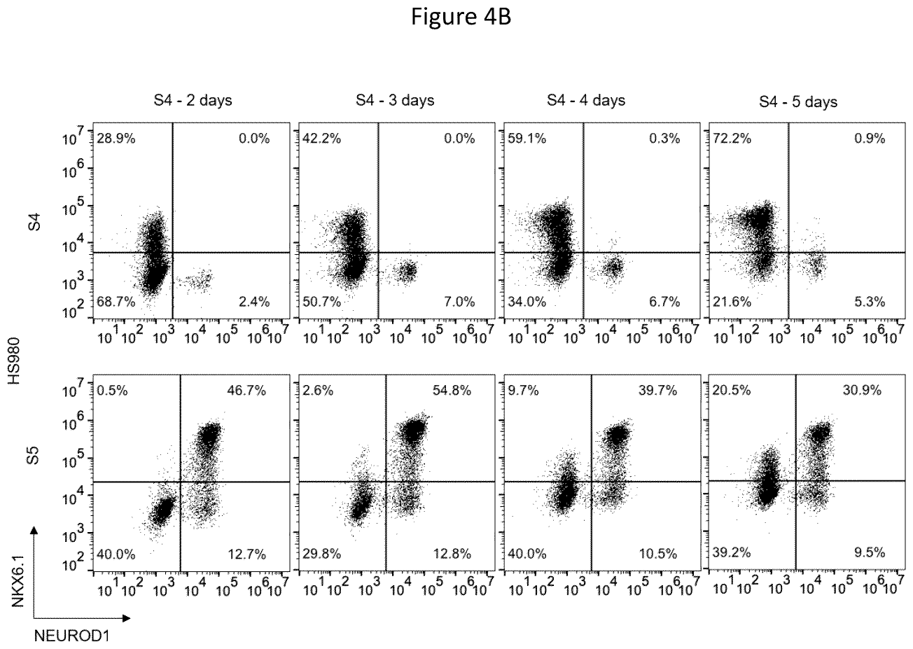

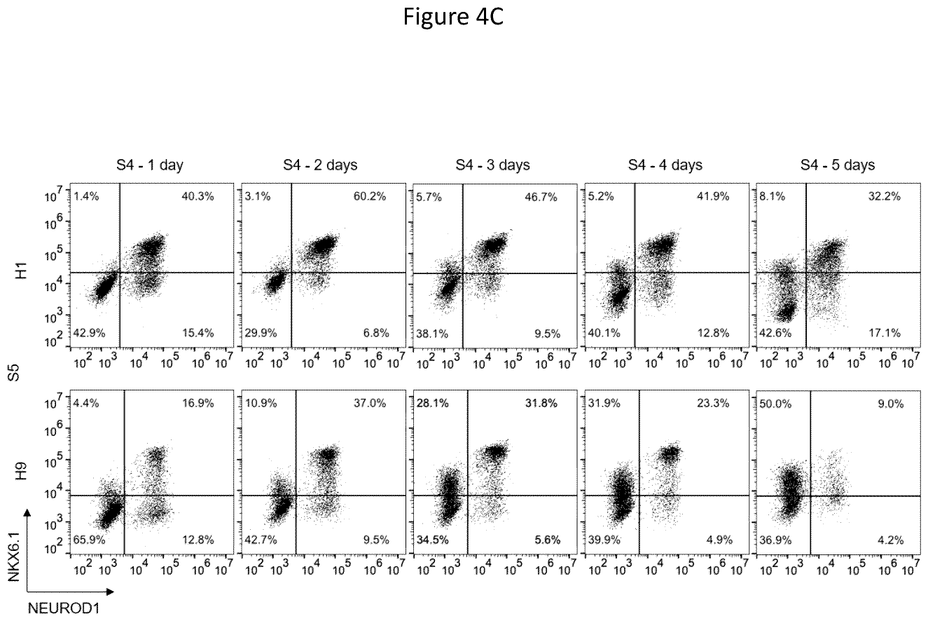

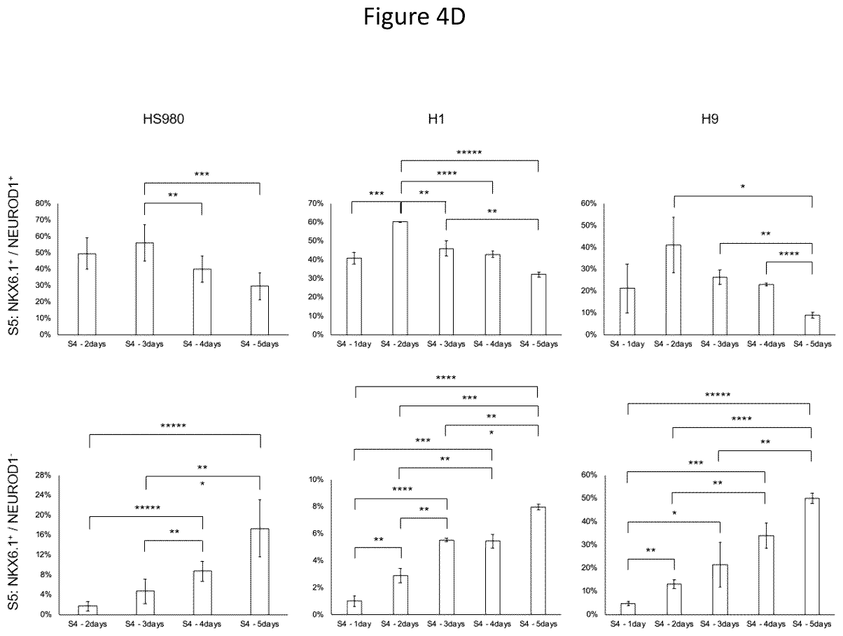

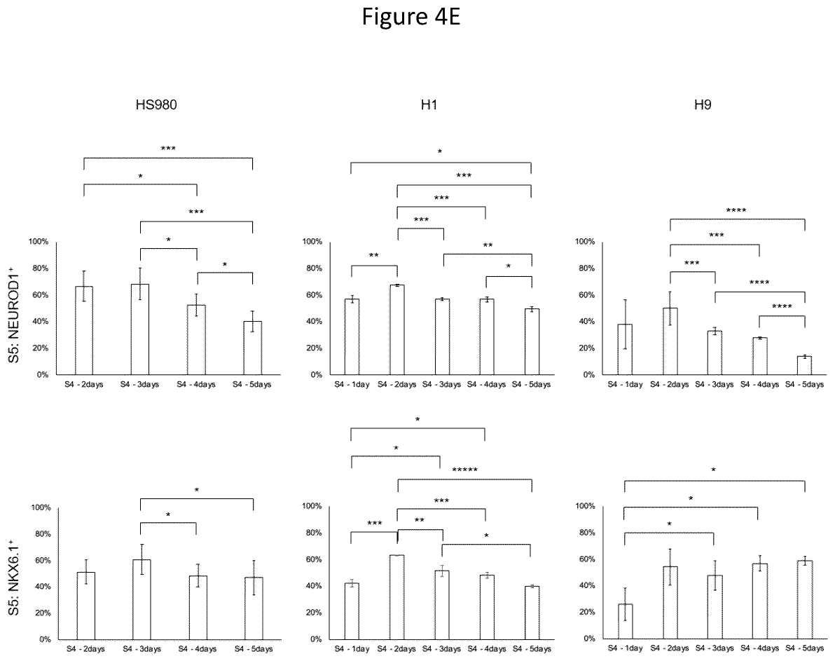

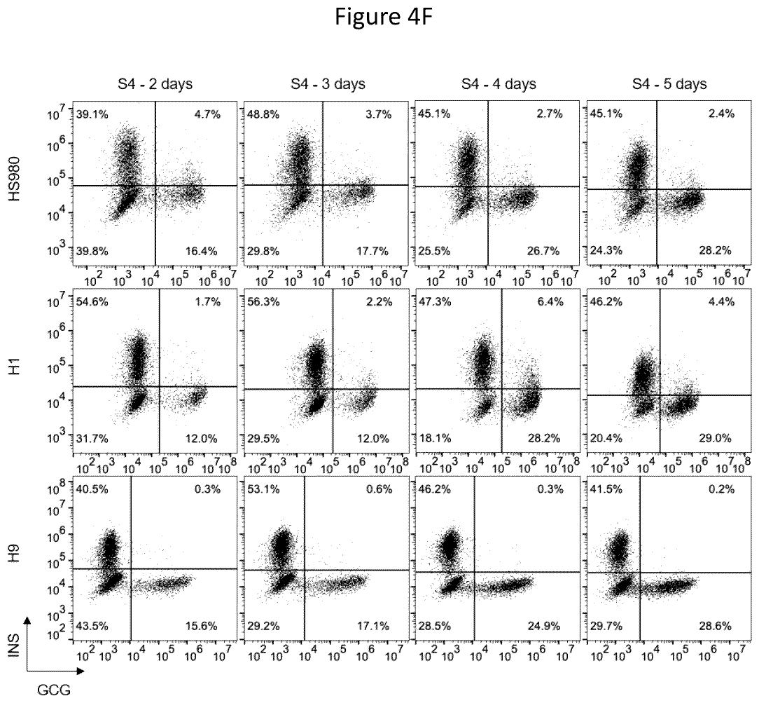

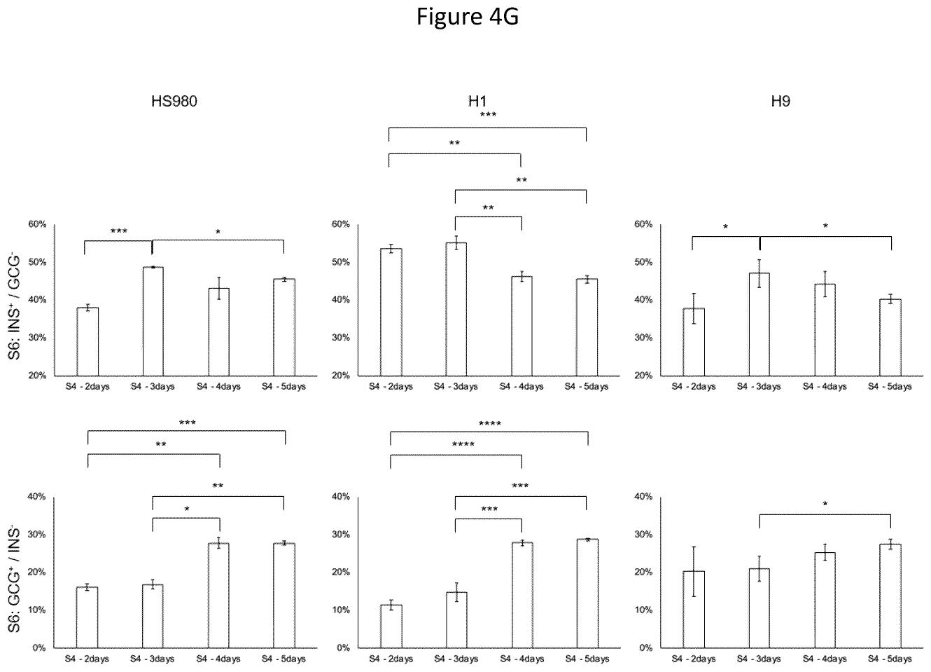

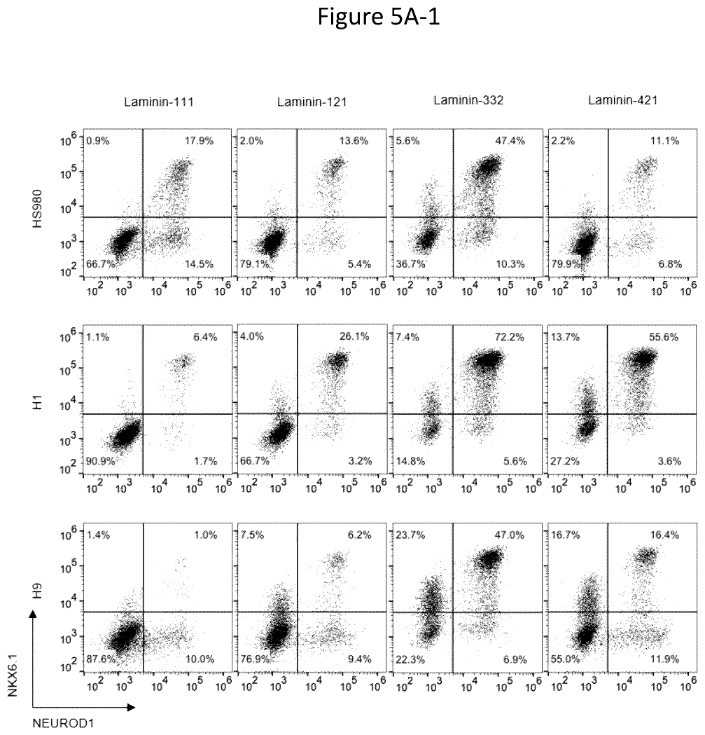

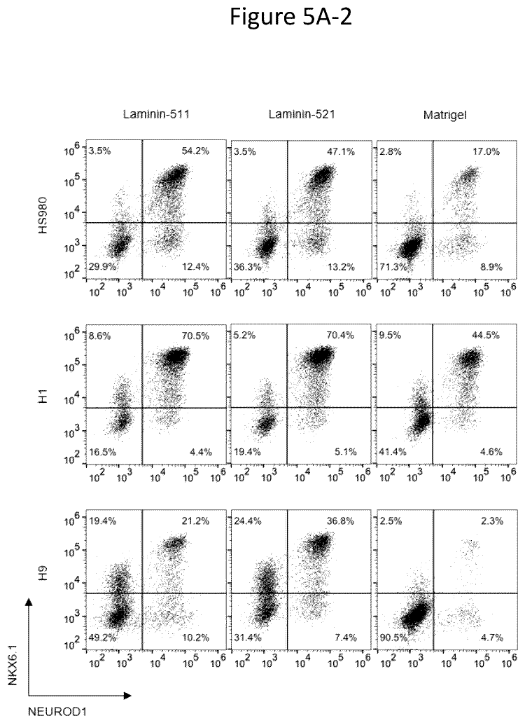

Effects of stage 4 duration on differentiation into endocrine progenitor cells To analyze the effects of S4 duration on S5 EP differentiation, HS980, H1, and H9 cells were induced for 1 day under S3 and then for 1, 2, 3, 4 or 5 days under S4 (as illustrated schematically in FIG. 4A).

Results: The expression of NKX6.1 and NEUROD1 were measured at the end of S4 and then four days into S5 (representative dot plots are shown in upper and lower panel, respectively, in FIG. 4B). The results showed a strong increase in the percentage of S4 NKX6.1⁺ PP cells when the S4 duration increased from 2 to 5 days for HS980 cells. In contrast, the percentage of S5 NKX6.1⁺/NEUROD1⁺ EP cells decreased as the duration of S4 increased (FIG. 4B). A similar inhibitory effect was also observed for both H1 and H9 cells. The percentage of NKX6.1⁺/NEUROD1⁺ EP cells began to decrease when the duration became longer than 2-3 days (FIG. 4C). The experiments were repeated several times and the results showed that 2-3 days under S4 were optimal for subsequent differentiation into S5 NKX6.1⁺/NEUROD1⁺ EP cells (FIG. 4D). In contrast, increased duration under S4 generated significantly higher percentage of NKX6.1⁺/NEUROD1-cells at S5 (FIG. 4D). It is likely that the NKX6.1⁺/NEUROD1-cells were actually S4 NKX6.1⁺ PP cells that failed to further differentiate into S5 NKX6.1⁺/NEUROD1⁺ EP cells due to the inhibitory effects of long duration (FIG. 4D). The inhibitory effects of long S4 duration were particularly obvious in H9 cells, and a reduction from 5 to 2 days increased the percentage of total NEUROD1⁺ endocrine cells to similar levels in HS980 and H1 cells (FIG. 4E). The results also showed that 1 day under S4 was not sufficient for induction of NKX6.1 (FIG. 4E).

Thus, it was concluded that the S4 duration of 2-3 days allow for higher percentage of NKX6.1⁺/NEUROD1⁺ EP cells and is therefore beneficial for the differentiation into S5 EP cells. The optimal S4 duration also minimizes cell line variability during pancreatic differentiation.

Effects of Stage 4 Duration on Maturation of Islet Cells

The effects of S4 duration on S6 islet cell maturation were also analyzed. To generate S6 islet-like cell aggregates, the S5 EP cells from different S4 durations were dissociated into single cells and then maintained in suspension as described above. The expression of key markers INSULIN (INS) and GLUCAGON(GCG) were analyzed at the end of S6.

Results: The results showed that the percentages of INS⁺ mono-hormonal β-cells decreased and the percentages of GCG⁺ mono-hormonal α-cells increased significantly when the S4 duration became longer than 3 days as shown in the representative dot plots (FIG. 4E) and bar graphs (FIG. 4F). It was noted that a 2 day duration of S4 resulted in high percentage of EP cells in cultures of H1 cells and also in cultures of H9 cells and that a 3 day duration of S4 resulted in high percentages of EP cells in H980 cultures (FIG. 4D). However, the duration of 3 days resulted the highest percentages of S6 INS⁺ beta cells (FIG. 4F).

In summary: Taken together, these results show that long S4 durations promoted differentiation into S4 NKX6.1⁺ PP cells but inhibited further differentiation into S5 NKX6.1⁺/NEUROD1⁺ EP cells. On the other hand, short S4 durations generated fewer S4 NKX6.1⁺ PP cells but increased S5 NKX6.1⁺/NEUROD1⁺ EP cells. The results also showed that S4 duration had opposite effects on α- and β-cell populations. Duration of 3 days under S4 promoted INS⁺β-cells and duration of 5 days strongly promoted GCG⁺ α-cells. Importantly, the results were confirmed in three different hESC lines (H1, H9 and HS980).

### Example 3

In this Example different cell culture coating substrates for pancreatic differentiation where evaluated.

Chemically defined and xenofree cell culture substrates such as LN-521 can provide more consistent and reliable conditions for expansion and differentiation of pluripotent stem cells.

TABLE 2
Cell adhesion on 2D surface coated with different matrix proteins.

| | LN- | LN- | LN- | LN- | LN- | LN- | LN- | LN- | LN- | |
|---|---|---|---|---|---|---|---|---|---|---|
| | 111 | 121 | 211 | 221 | 322 | 411 | 421 | 511 | 521 | Matrigel |
| HS980 | + | + | − | − | + | − | + | + | + | + |
| H1 | + | + | − | − | + | − | + | + | + | + |
| H9 | + | + | − | − | + | − | + | + | + | + |

HS980, H1 and H9 cells were passaged onto plates coated with different recombinant human Laminin isoforms (LN) or Matrigel as shown in the table.
Cell adhesion and survival were examined under microscope after 3-4 days.
+ indicated that the cells attached and formed monolayers on the bottom, − indicates that the cells failed to attach to the coated surface.

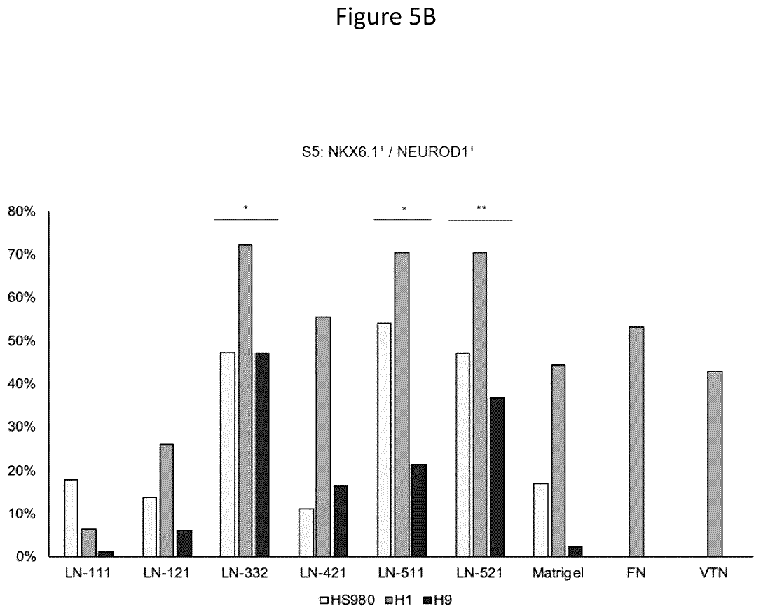

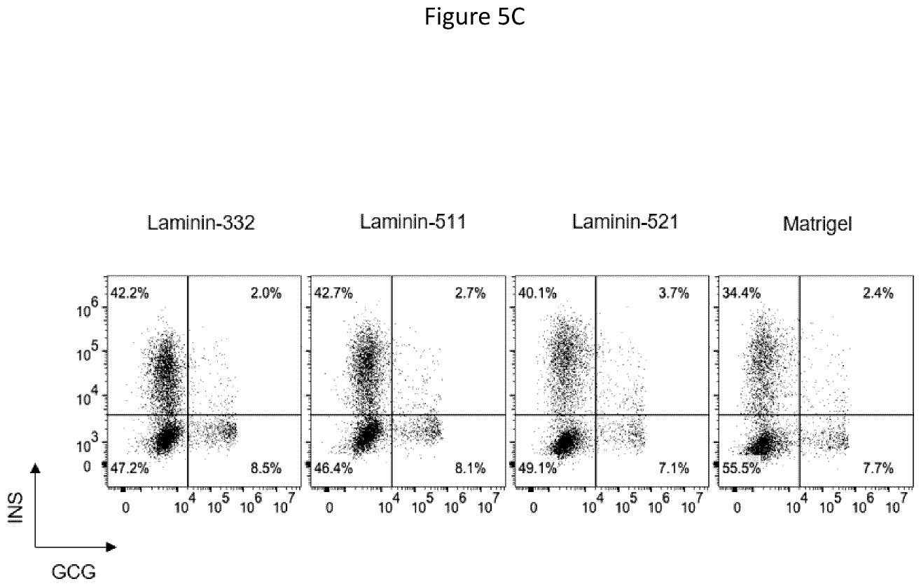

To determine if hESCs can also be differentiated into pancreatic endocrine cells on other matrix proteins, we passaged HS980, H1, and H9 cells onto cell culture plates coated with Matrigel (1/100, Corning; 354277), or human recombinant LN-111, LN-121, LN-211, LN-221, LN-332, LN-411, LN-421, LN-511, or LN-521 (all obtained from BioLamina). HS980, H1, and H9 cells attached to LN-111, LN-121, LN-332, LN-421, LN-511, LN-521, and Matrigel (Table 2). The cells were differentiated towards S5 EP cells using the short protocol and the expression of NKX6.1 and NEUROD1 were analyzed four days into stage 5 (FIG. 5A). The results showed that LN-332, LN-521, and LN-511 supported differentiation into S5 NKX6.1⁺/NEUROD1⁺ EP cells more efficiently than Matrigel (FIG. 5A, B). Notably, LN-511 enhanced S5 EP differentiation as efficiently as LN-521 for both HS980 and H1 cells when the short protocol was used instead of the long protocol (FIG. 5A, B). H1 cells were also differentiated on plate coated with 10 μg/mL Fibronectin (FN, Sigma; F0895) or Vitronectin (VTN, Sigma; 5051). The results showed that FN and VTN were able to support S5 EP differentiation (FIG. 5B).

S5 EP cells generated on LN-332, LN-511, LN-521, and Matrigel were chosen for further differentiation towards S6 islet cells as described above. The expression of INS and GCG were measured at the end of S6. The results showed that S5 EP cells from these substrates were able to generate S6 mono-hormonal INS⁺ B cells and GCG⁺ a cells (FIG. 5C).

Taken together, the results showed that the short protocol was highly efficient on multiple xenofree defined substrates.

### Example 4

In this Example, it was investigated whether S4 or S5 cells are more suitable for the formation of islets in vitro.

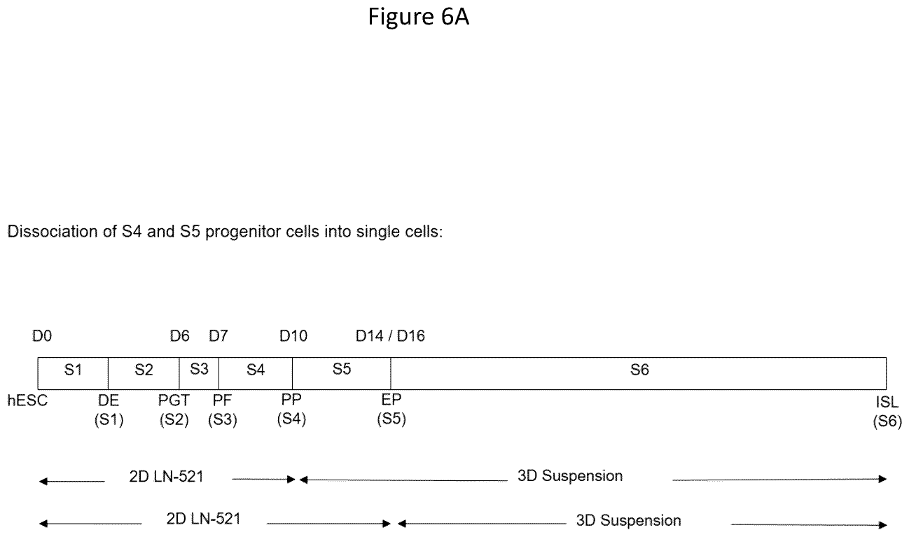

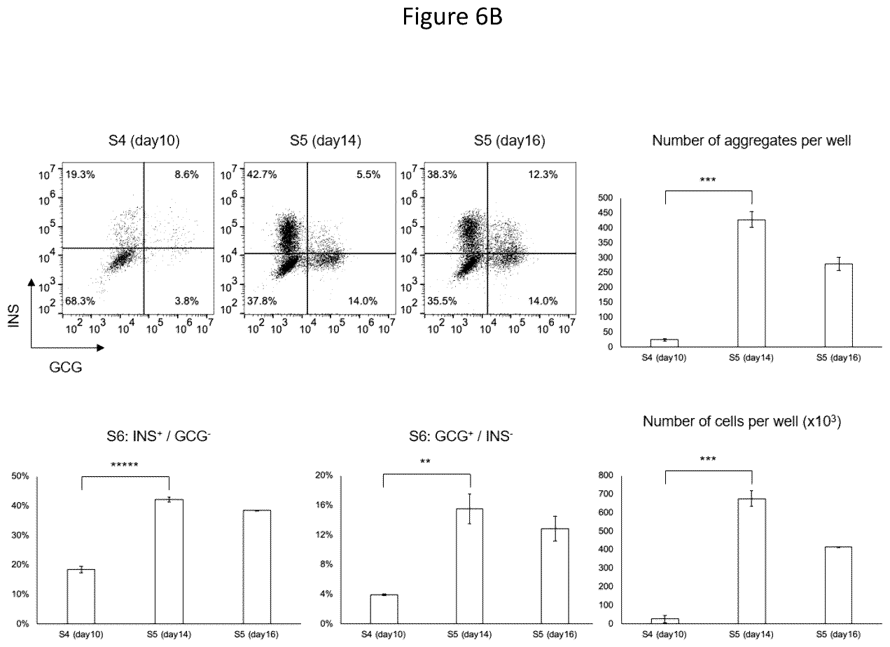

In previous reports purified S4 PP cells have been used for generation of pancreatic islets (Cogger et al, 2017; Ameri et al., 2017; Kelly et al., 2011). To determine which cell population is more suitable for islet formation, HS980 cells were dissociated into single cells at the end of S4 or 4 days into S5. The cells were then maintained in suspension to generate islet-like aggregates (FIG. 6A). The expression of key endocrine markers INS and GCG, numbers of islet-like aggregates, and numbers of islet cells were analyzed at the end of S6.

Results: The results showed that aggregates generated from S5 EP cells had much higher percentages of both INS⁺β-cells and GCG⁺α-cells than the aggregates from S4 PP cells (FIG. 6B). S5 EP cells also generated over 10 times more islet-like aggregates and islet cells than S4 PP cells (FIG. 6B).

To determine if a longer S5 duration improves islet formation, the S5 EP cells were dissociated into single cells 6 days into S5 and then maintained in 3D suspension. However, the percentages of a and β-cell remained unchanged and the numbers of aggregates/cells rather decreased at S6 (FIG. 6B). Taken together, these data show that the S5 EP cells but not the S4 PP cells efficiently formed islet-like aggregates in 3D suspension, and a duration of 4 days under S5 was sufficient for aggregate formation in suspension.

### Example 5

In this Example, the effect of culture of cells in a 3D single cell suspension was investigated.

To determine if 3D single cell suspension selectively promoted formation of aggregates from S5 EP cells, HS980 and H1 cells were differentiated into S5 EP cells on LN-521 and then dissociated into single cells in 3D suspension.

The expression of NKX6.1, NEUROD1, and cell proliferation marker Ki-67 were analyzed before and after aggregate formation in 3D suspension.

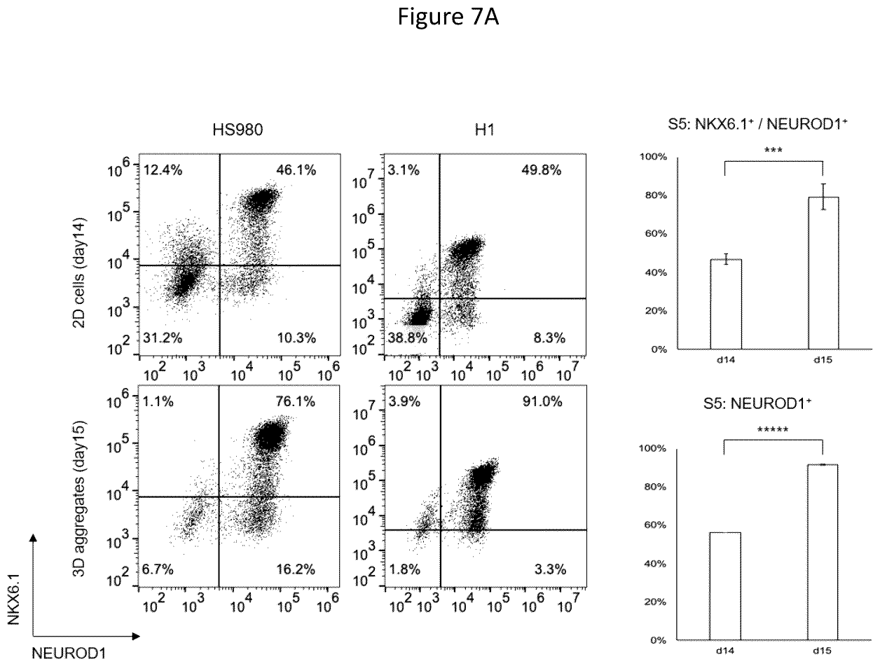

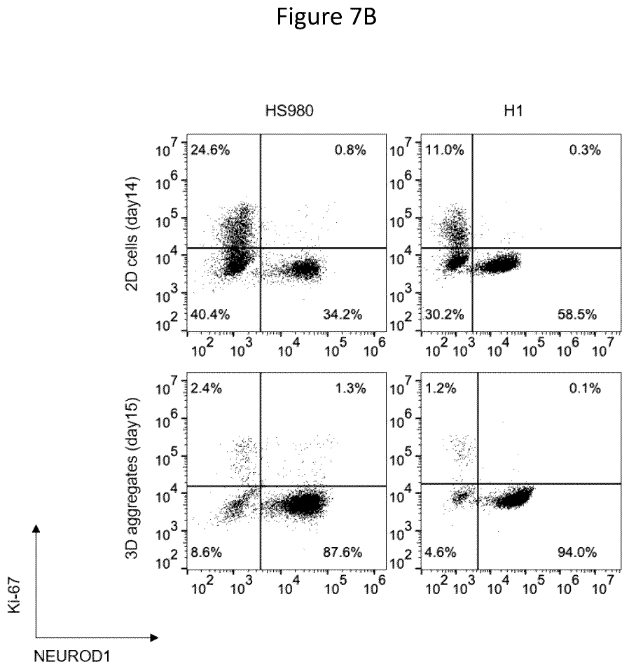

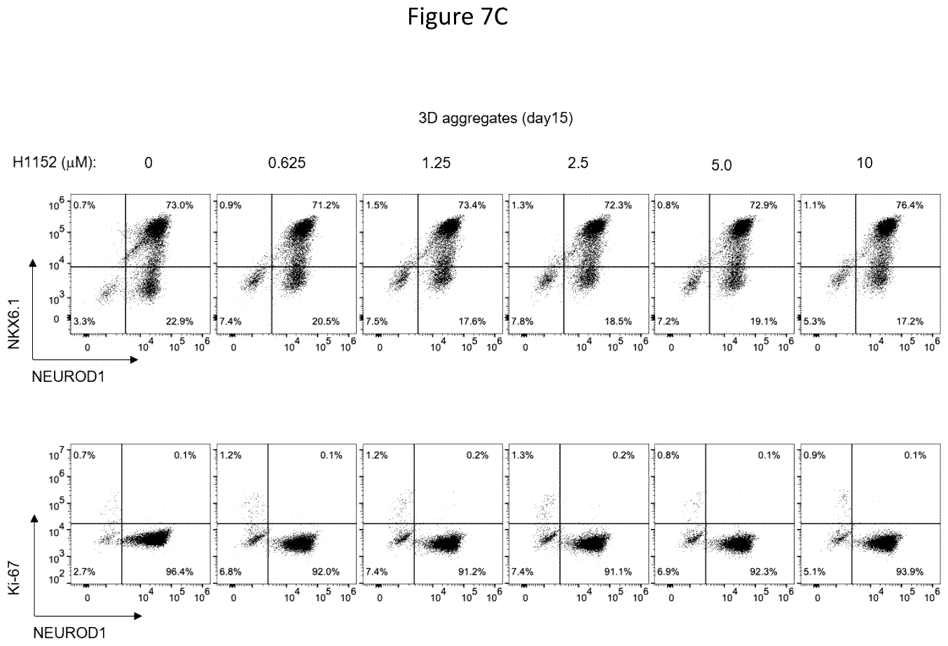

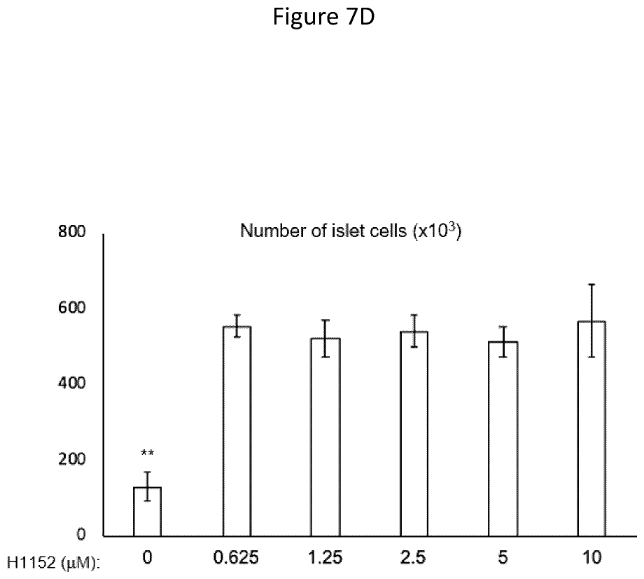

Results: The results show a clear increase in the percentages of S5 NKX6.1⁺/NEUROD1⁺ and total NEUROD1⁺ cells in the newly formed aggregates and strong decrease in the percentages of NEUROD1-non-endocrine cells in 3D aggregates at day 15 compared to 2D cells at day 14 (FIG. 7A). The results also showed that S5 NEUROD1⁺ EP cells are non-proliferative Ki-67-cells, and most of the proliferative Ki-67⁺/NEUROD1⁻ and non-proliferative Ki-67−/NEUROD1-cells were removed during formation of aggregates in 3D suspension (FIG. 7B).

To determine if ROCK inhibitor was required for S5 EP cell survival and aggregate formation, different concentrations of H1152, from 0 to 10 μM, were added to the 3D single cell suspension for one day. The expression of NKX6.1, NEUROD1, and Ki-67, and the aggregate cell numbers were measured one day after aggregates formation in 3D suspension.

Results: The results showed that the concentration of H1152 had no effect on the percentages of S5 EP cells in the newly formed aggregates (FIG. 7C). However, the numbers of islet cells increased significantly in the presence of low concentrations of H1152, indicating that H1152 promoted survival of S5 EP cells as single cells in suspension (FIG. 7D).

Taken together, these results showed a selective enrichment of S5 EP cells due to the 3D aggregate formation from single cells, in agreement with previous studies of fetal pancreas development(Gouzi et al., 2017).

### Example 6

In this example a comparison of short and long pancreatic differentiation protocols was made by comparing the percentages of pancreatic progenitor (PP) cells and endocrine progenitor (EP) cells obtained.

Next, we compared the short and long protocols. HS980 cells on LN-521 were differentiated towards S5 EP cells using the short and long differentiation protocols as described above (illustrated schematically in FIG. 1B). The expression of key progenitor markers was analyzed at the 5 end of S3, S4 and four days into S5 (differentiation day 7, 10 and 14 for the short protocol, and 8, 13 and 17 for the long protocol).

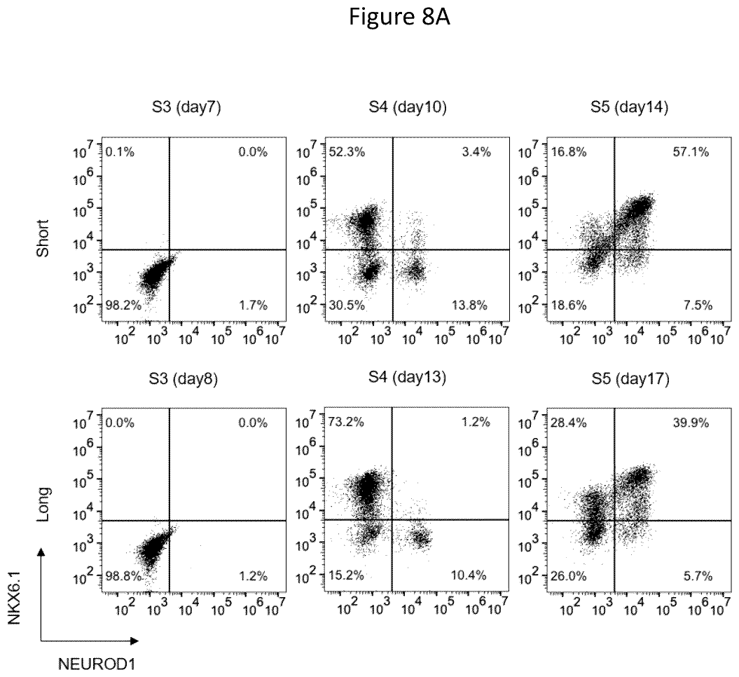

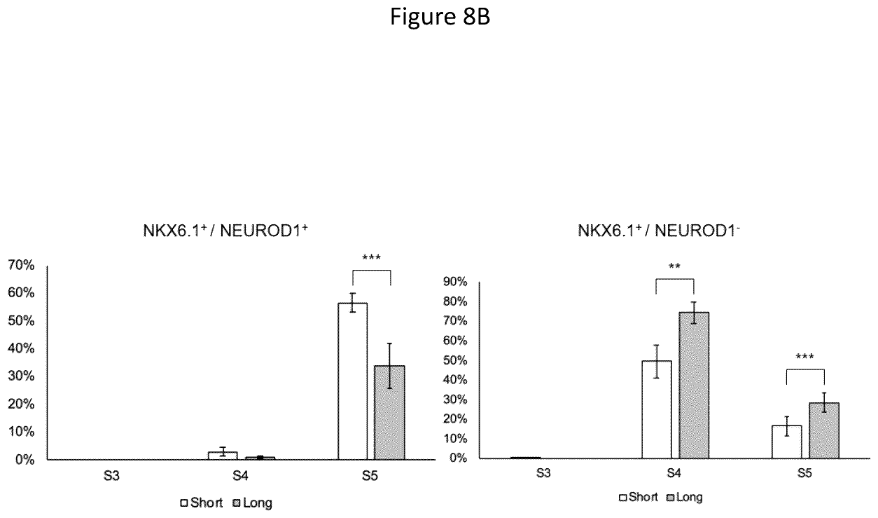

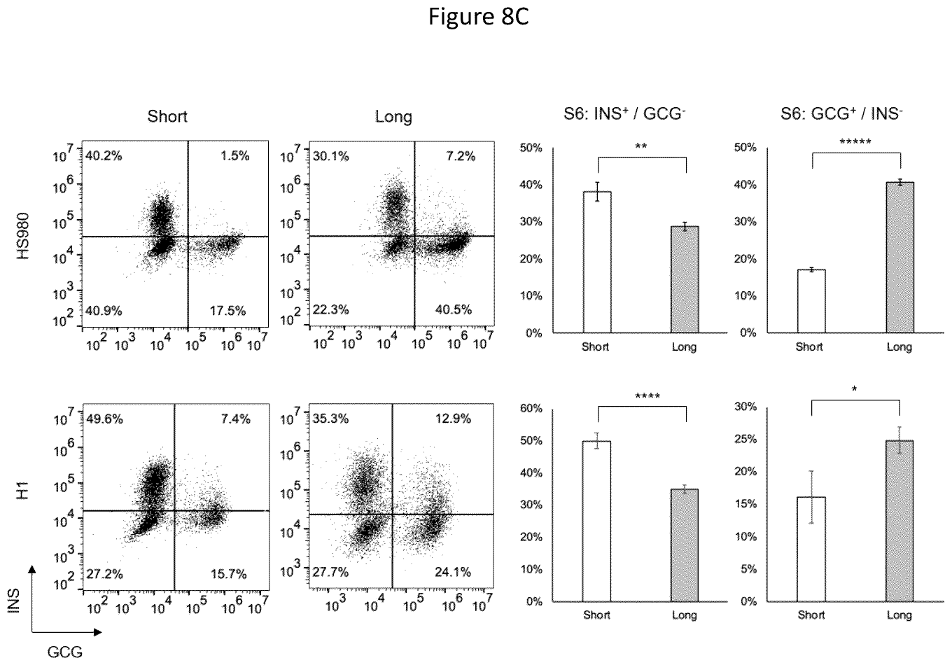

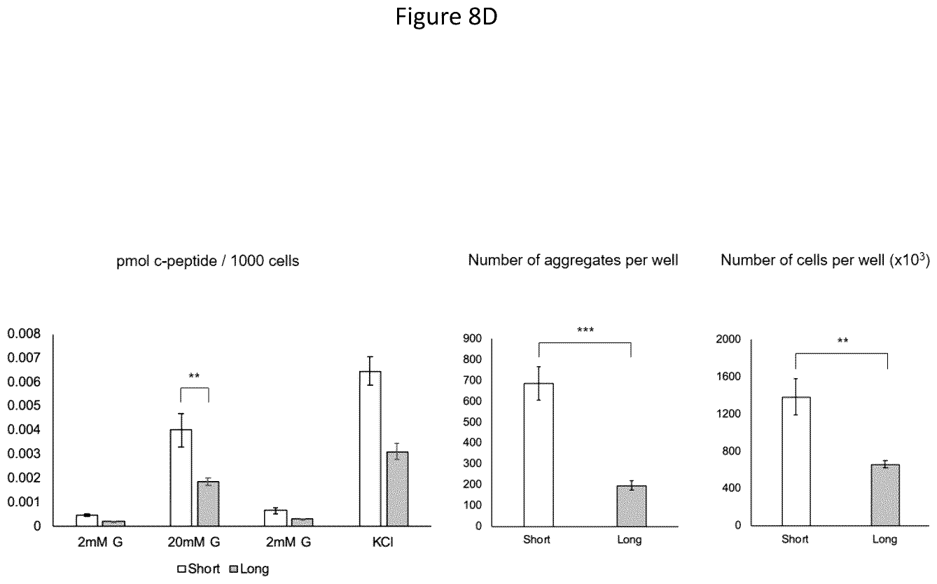

Results: The results showed opposite effects of the short and long protocols. The short protocol generated lower percentages of S4 NKX6.1⁺ PP cells but higher percentages of S5 NKX6.1⁺/NEUROD1⁺ EP cells than the long protocol (FIGS. 8A and B). The percentages of NKX6.1⁺/NEUROD1-non-endocrine cells at S5 were lower for the short protocol than for the long protocol (FIGS. 8A and B). These results together indicate a positive effect of the short S3+S4 duration on S5 EP differentiation.

To determine if S3+S4 duration also affects islet maturation during S6, the S5 EP cells were dissociated into single cells and then maintained in suspension as described above. The expression of key endocrine markers INS and GCG, numbers of islet-like aggregates and islet cells, and in vitro glucose stimulated insulin secretion were analyzed at the end of S6 (FIGS. 8C and D). Results: The results showed that the short protocol induced higher percentage of INS⁺ mono-hormonal β-cells but lower percentage of GCG+ mono-hormonal α-cells than the long protocol (FIG. 8C). Similar effects of S3+S4 duration were also observed in H1 cells (FIG. 8C). The short protocol also generated much higher numbers of islet-like aggregates and islet cells than the long protocol (FIG. 8D). The results from in vitro glucose stimulation experiments confirmed that the aggregates at the end of S6 were fully functional and could increase insulin secretion by 10 folds in response to high glucose concentration, but the aggregates generated using the short protocol secreted more insulin at high glucose level (FIG. 8D). Taken together, these results showed strong positive effects of the short S3+S4 duration on islets maturation during S6.

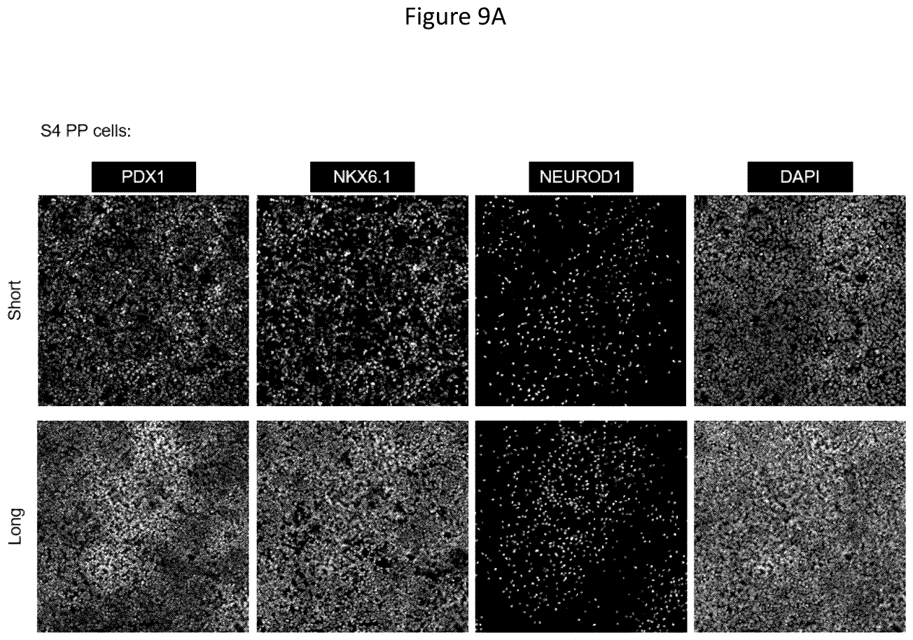

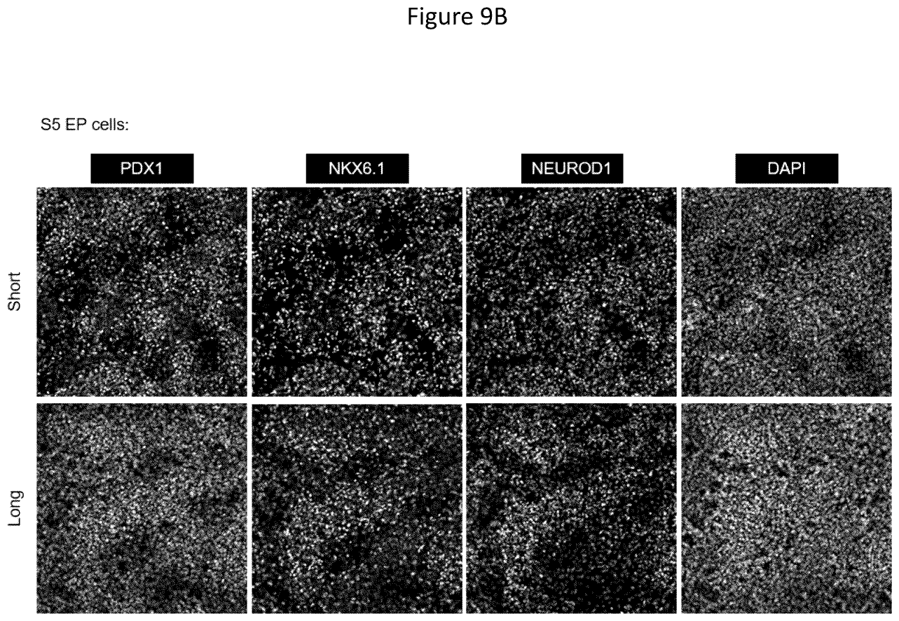

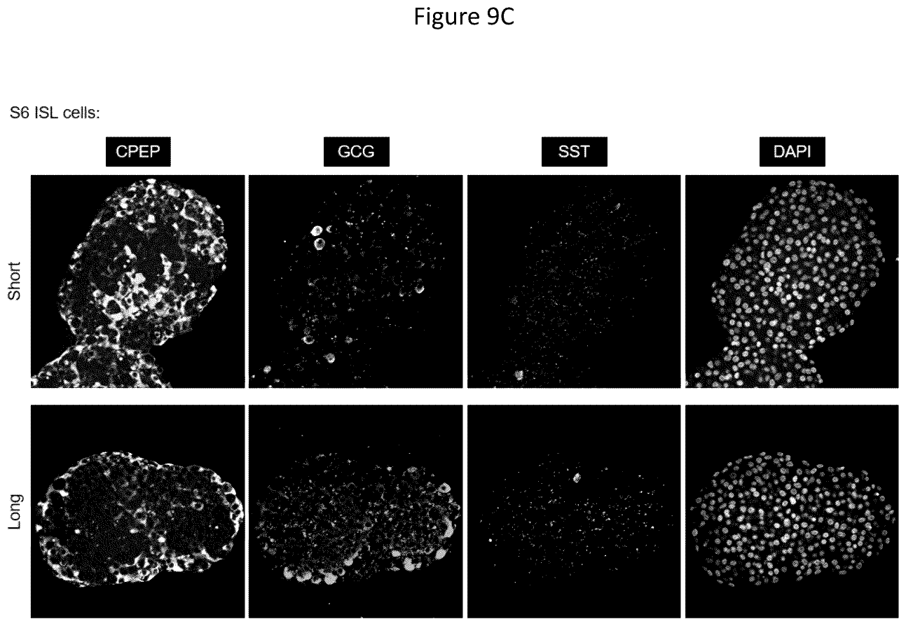

The expression of stage specific markers was also analyzed using immunocytochemistry (ICC) (FIG. 9). The long protocol induced a very dense layer containing mainly PDX1+/NKX6.1⁺ PP cells at S4 (FIG. 9A). In contrast, the short protocol induced less S4 PDX1+/NKX6.1⁺ PP cells and some cells remained NKX6.1−(FIG. 9A). Pre-mature induction of NEUROD1 in NKX6.1-cells at S4 was also observed for both protocols (FIG. 9A). Further differentiation into S5 EP cells showed opposite effects. The short protocol generated more S5 NKX6.1⁺/NEUROD⁺ EP cells than the long protocol (FIG. 9B). The results also showed that aggregates generated using the short and long protocols had different ratios between β and α-cells (FIG. 9C). The short protocol induced more INSULIN c-peptide (CPEP) positive β-cells and the long protocol induced more GCG⁺α-cells (FIG. 9C).

A few SOMATOSTATIN (SST) positive 8 cells were also observed (FIG. 9C). Taken together, the results from ICC supported the conclusions from flow cytometry analysis described above.

### Example 7

In this Example, the effect of spontaneous aggregation of S5 cells into 3D cultures compared to forced aggregation using AggreWell 400 plates and 96-well plates on the generation of monohormonal pancreatic β-cells is investigated.

For spontaneous aggregation, H1 cells were dissociated into single cells at S5 and maintained in 3D suspension as described above. For forced aggregation in microwells, the S5 cells were transferred to AggreWell or 96-well plates instead. The identity of the cells generated was investigated by flow cytometry one day after 3D aggregation and again at the end of S6.

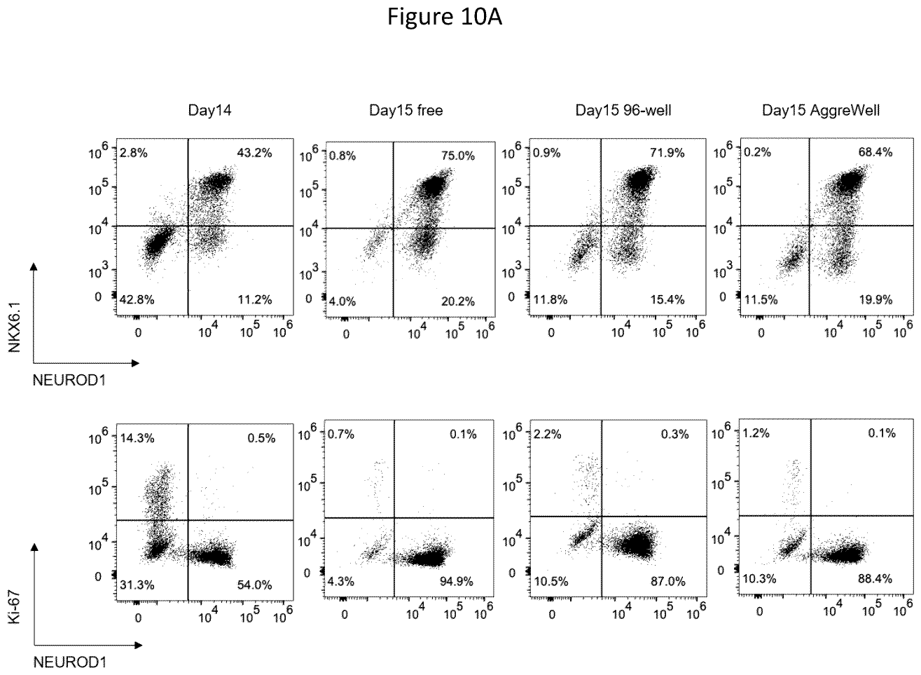

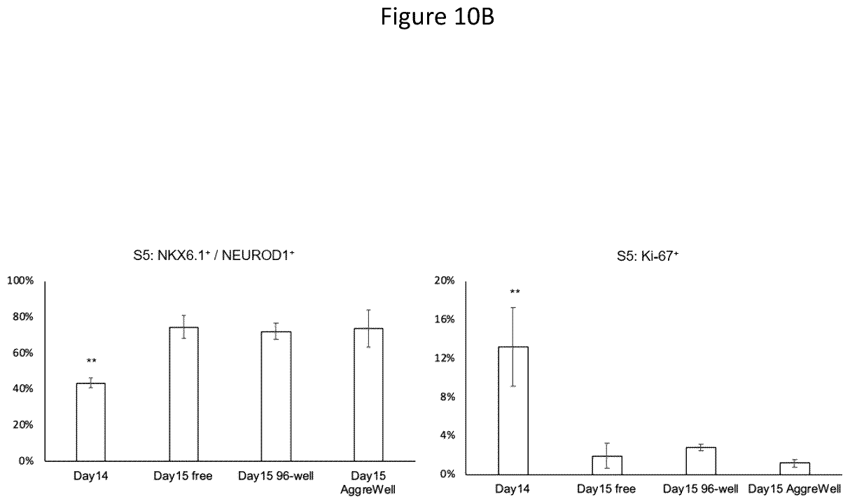

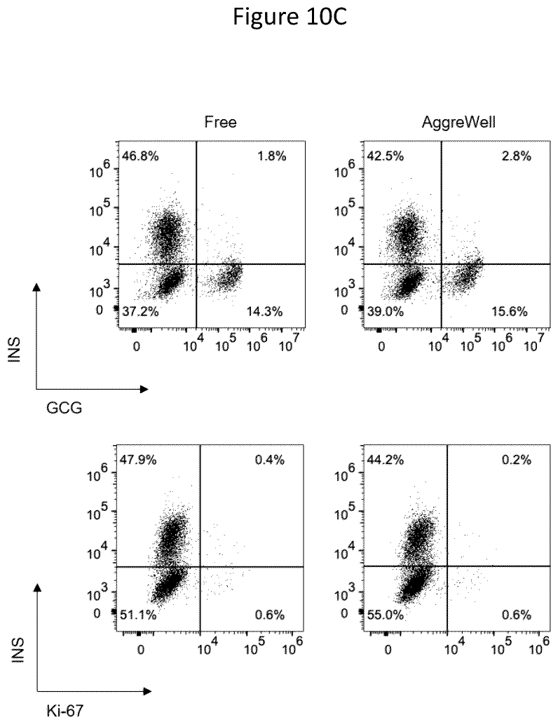

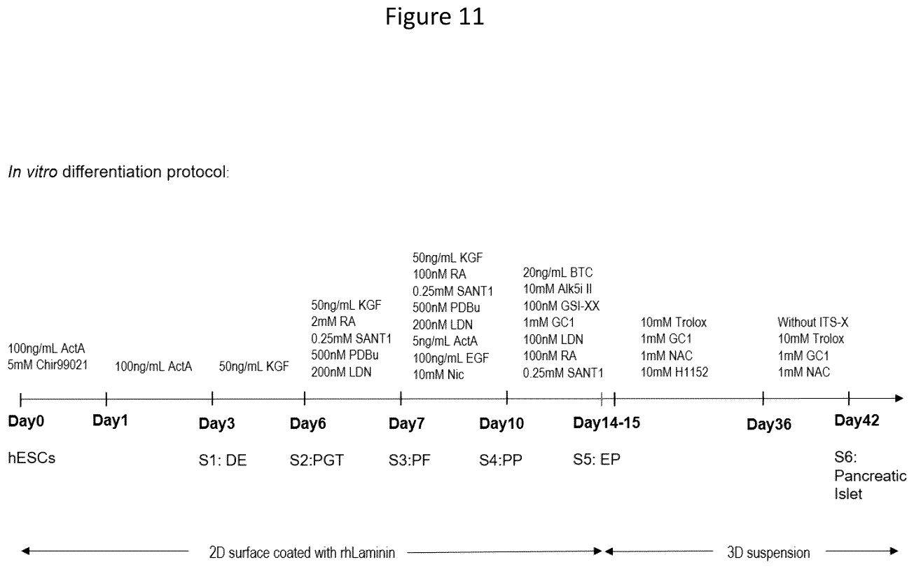

Results: The results showed that aggregates generated in microwells contained mainly NKX6.1⁺/NEUROD1⁺/Ki-67-EP cells as the aggregates generated spontaneously in 3D suspension culture (FIG. 10A, B). The proliferative Ki-67⁺ cells were removed from the aggregates in microwells as in 3D suspension (FIG. 10A, B). At the end of S6, the aggregates generated in microwells contained similar percentages of INS⁺β-cells and GCG⁺α-cells as the aggregates generated spontaneously (FIG. 10C). Only very few Ki-67⁺ proliferative cells still remained in these aggregates (FIG. 10C). Taken together, these results indicate that S5 EP cells can dissociate themselves from other non-endocrine cell types and then undergo spontaneous self-aggregation.

### Example 8

In this example, cell line variability was evaluated in multiple hPSC lines differentiated using the short pancreatic differentiation protocol.

The hESC lines HS980, H1 and H9 were differentiated into S6 islet cells as described above. The expression of key markers INS and GCG, and in vitro glucose stimulated insulin secretion were analyzed at the end of S6. To determine if the short differentiation protocol also works in human iPSC lines, C7 cells were differentiated on LN-521 and the expression of key markers were examined at the end of S5 and S6.

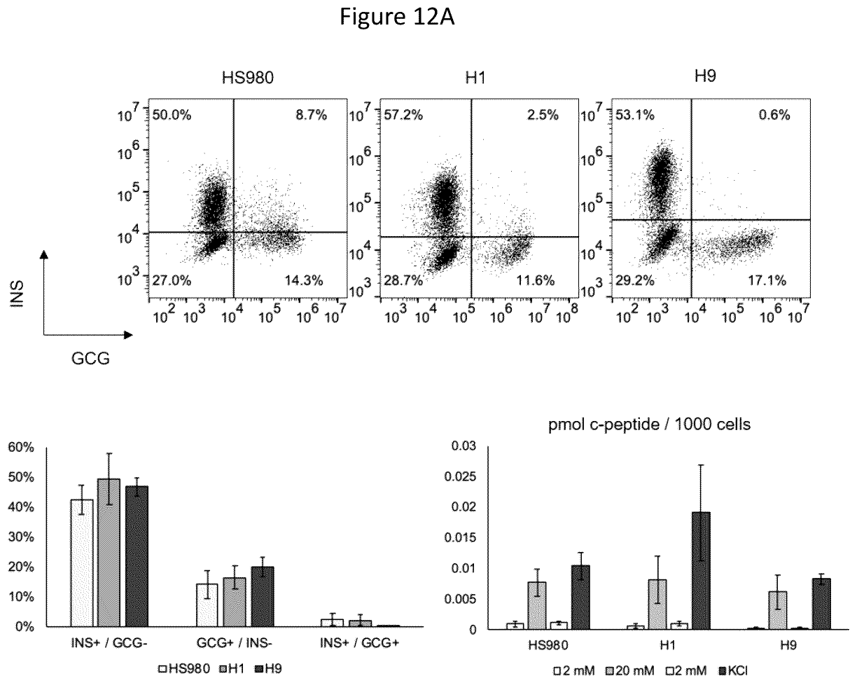

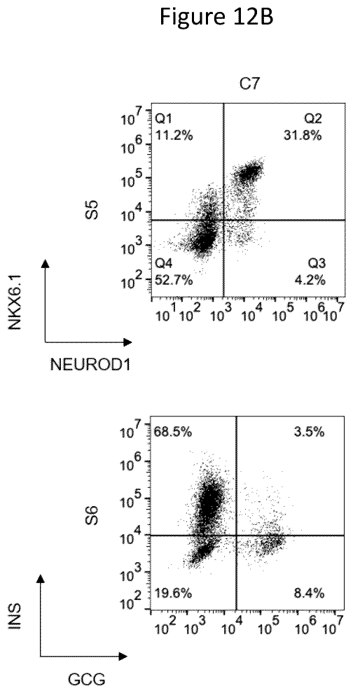

Results: The results showed that the percentages of mono-hormonal INS⁺ B cells and GCG⁺α cells were comparable among all three hESC lines, and the aggregates were functional and could increase insulin secretion in response to high glucose concentration in a similar manner (FIG. 12A). The results also showed that human iPSC lines could be successfully differentiated into S5 NKX6.1⁺/NEUROD1⁺ EP cells and subsequently into S6 mono-hormonal INS⁺β cells and GCG⁺α cells (FIG. 12B).

Taken together, these results showed relatively low cell line variability among multiple human ESC and iPSC lines differentiated using the short pancreatic differentiation protocol.

### Example 9

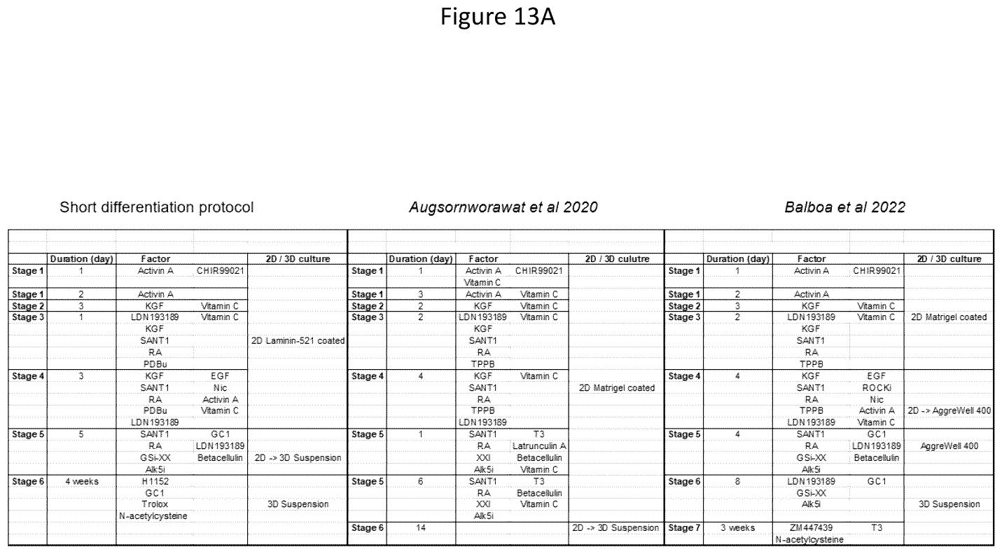

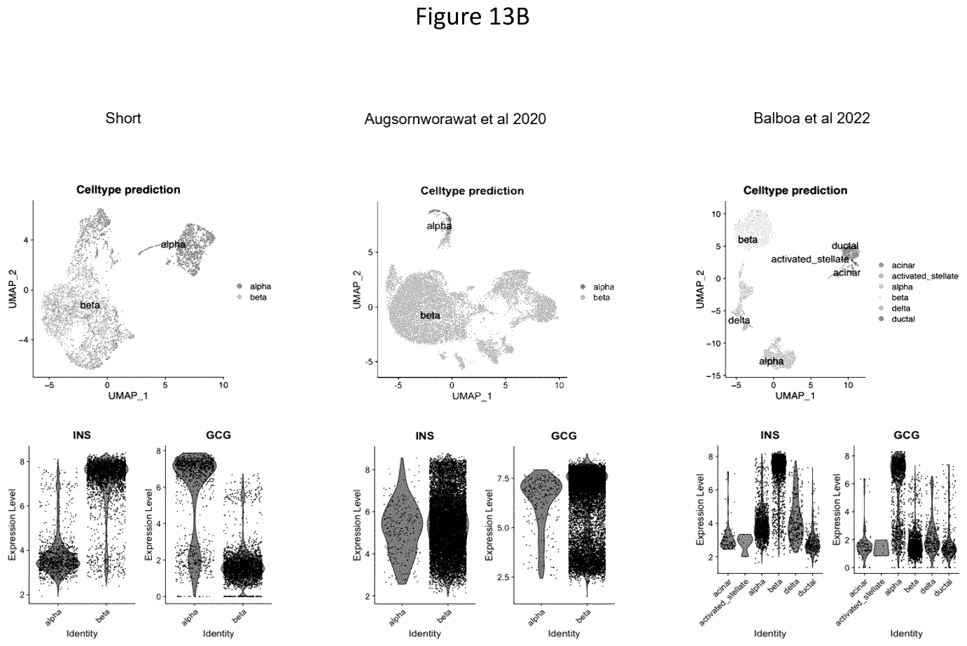

In this example, the short pancreatic differentiation protocol was compared to two previously published protocols (Augsornworawat et al 2020, and Balboa et al 2022) using datasets from single-cell RNA sequencing (scRNAseq) experiments (FIG. 13A).

H1 cells were differentiated into S6 islet cells using the short pancreatic differentiation protocol as described above. The gene expression profiles of the islet cells were investigated by single-cell RNA sequencing at the end of S6. The dataset obtained was processed and visualized by UMAP projection (FIG. 13B). The cell type identity and expression of key marker genes in each cell populations were analyzed (FIG. 13B). Two published datasets (Augsornworawat et al 2020, and Balboa et al 2022) were also included in the analysis.

Results: The results showed that S6 islets generated using the short differentiation protocol contained two mature mono-hormonal cell populations, INS expressing B cells and GCG expressing a cells (FIG. 13B, left). The islets generated by Augsornworawat et al also contained two main endocrine cell populations (FIG. 13B, middle). However, most of the cells co-expressed both INS and GCG and were therefore immature polyhormonal β and a cells. In contrast, the islets from Balboa et al contained both endocrine and non-endocrine cell types although the predcited β and α were mono-hormonal (FIG. 13B, right).

Taken together, these results showed that the islets generated with the short pancreatic differentiation protocol contained mainly mature monohormonal β and a cells. In contrast, two recent published protocols generated islets containing either immature polyhormonal cells or non-endocrine cells.

### Example 10

In this example S5 EP cells, frozen and not frozen, were compared for their abilities to generate 3D islets.

HS980 and H1 cells were differentiated towards S5 as described above. The cells were dissociated into single cells in 3D suspension and then further differentiated towards S6. Alternatively, the S5 single cells were frozen and kept in liquid nitrogen. The frozen cells were later thawed and differentiated towards S6 as described above. The identity of the islet cells and in vitro glucose stimulated insulin release were examined at the end of S6.

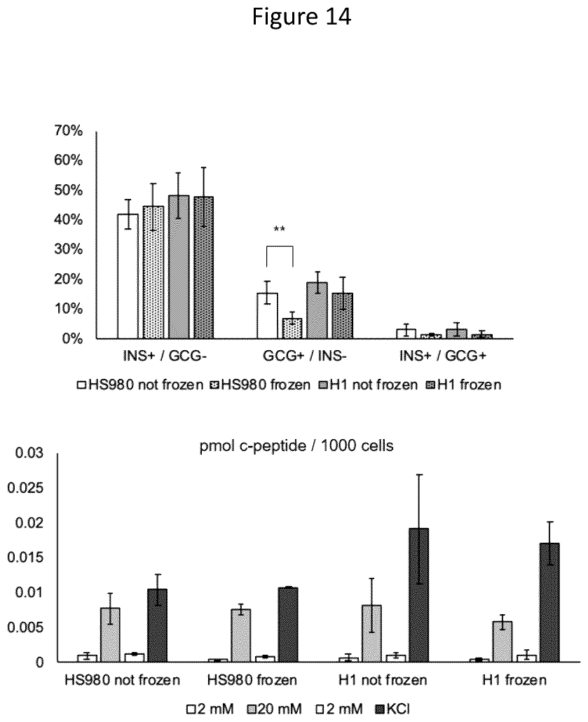

Results: The results showed that the freezing/thawing procedure at S5 did not change the percentage of INS⁺β cells in HS980 and H1 cells (FIG. 14, left). The percentage of GCG⁺α cells decreased significantly in HS980 cells but not H1 cells after the freezing/thawing procedure (FIG. 14, left). The results also showed that islets differentiated from S5 EP cells, both frozen and not frozen, were functional and could increase insulin release in responsive to high glucose level (FIG. 14, right).

Taken together, these results showed that the S5 EP cells can be cryopreserved and the frozen cells are ideal resource for production of functional islets in vitro.

### REFERENCES

1. Rodin, S., et al., Clonal culturing of human embryonic stem cells on laminin-521/E-cadherin matrix in defined and xeno-free environment. Nat Commun, 2014. 5: p. 3195.
2. Pagliuca, F. W., et al., Generation of functional human pancreatic β-cells in vitro. Cell, 2014. 159 (2): p. 428-39.
3. Rezania, A., et al., Reversal of diabetes with insulin-producing cells derived in vitro from human pluripotent stem cells. Nat Biotechnol, 2014. 32 (11): p. 1121-33.
4. Millman, J. R., et al., Generation of stem cell-derived β-cells from patients with type 1 diabetes. Nat Commun, 2016. 7: p. 11463.
5. Vegas, A. J., et al., Long-term glycemic control using polymer-encapsulated human stem cell-derived beta cells in immune-competent mice. Nat Med, 2016. 22 (3): p. 306-11.
6. D'Amour, K. A., et al., Production of pancreatic hormone-expressing endocrine cells from human embryonic stem cells. Nat Biotechnol, 2006. 24 (11): p. 1392-401.
7. Kroon, E., et al., Pancreatic endoderm derived from human embryonic stem cells generates glucose-responsive insulin-secreting cells in vivo. Nat Biotechnol, 2008. 26 (4): p. 443-52.
8. Nostro, M. C., et al., Efficient generation of NKX6-1+ pancreatic progenitors from multiple human pluripotent stem cell lines. Stem Cell Reports, 2015. 4 (4): p. 591-604.
9. Cogger, K. F., et al., Glycoprotein 2 is a specific cell surface marker of human pancreatic progenitors. Nat Commun, 2017. 8 (1): p. 331.
10. Ameri, J., et al., Efficient Generation of Glucose-Responsive Beta Cells from Isolated GP2. Cell Rep, 2017. 19 (1): p. 36-49.
11. Gouzi, M., et al., Neurogenin3 initiates stepwise delamination of differentiating endocrine cells during pancreas development. Dev Dyn, 2011.

240 (3): p. 589-604.
12. Augsornworawat, P., et al., Single-Cell Transcriptome Profiling Reveals β Cell Maturation in Stem Cell-Derived Islets after Transplantation. Cell Rep, 2020. 32 (8): p. 1-13.
13. Balboa, D., et al., Functional, metabolic and transcriptional maturation of human pancreatic islets derived from stem cells. Nat Biotechnol, 2022. 40(7): p. 1042-55.
14. Kele, M., et al., Generation of human iPS cell line CTL07-II from human fibroblasts, under defined and xeno-free conditions. Stem Cell Res. 2016 November; 17 (3): p. 474-478.

Itemized list of embodiments
1. Method for the generation pancreatic islet-like cell aggregates in vitro, comprising the steps of
   i) providing a population comprising endocrine progenitor (EP) cells, such as a population of EP cells, such as EP cells characterized by the expression of NEUROD1; such as EP cells characterized by the expression of NKX6.1 and NEUROD1;
   ii) providing a single cell suspension of said population of EP cells;
   iii) allowing said population of EP cells in single cell suspension to form 3D structures;
   iv) culturing said population of EP cells in the form of 3D structures in 3D culture conditions permissive of differentiation pancreatic monohormonal β-cells to provide pancreatic islet-like cell aggregates; and
   v) thereby generating pancreatic islet-like cell aggregates comprising monohormonal β-cells, wherein said pancreatic islet-like cell aggregates comprise at least approximately 25%, such as at least approximately 30%, such as at least approximately 35%, such as at least approximately 40%, such as at least approximately 45%, monohormonal β-cells.
2. Method for the generation pancreatic islet-like cell aggregates in vitro according to item 1, wherein the population of EP cells in step i) is an adherent culture of EP cells on a 2D substrate.
3. Method for the generation pancreatic islet-like cell aggregates in vitro according to item 2, wherein said 2D substrate comprises one or more components selected from the group consisting of laminins (LN) and fragments thereof, vitronectin and fragments thereof, fibronectin and fragments thereof, collagen and fragments thereof, gelatin and fragments thereof, functionalized silk (FN silk) and Matrigel™, such as one or more components selected from the group consisting of laminins (LN) and fragments thereof, vitronectin and fragments thereof, fibronectin and fragments thereof and Matrigel™.
4. Method for the generation pancreatic islet-like cell aggregates in vitro according to item 3, wherein said laminins (LN) and fragments thereof are selected from the group consisting of LN-521 and fragments thereof, LN-511 and fragments thereof, LN-332 and fragments thereof, LN-421 and fragments thereof, LN-121 and fragments thereof and LN-111 and fragments thereof; such as the group consisting of LN-521 and fragments thereof, LN-511 and fragments thereof, LN-332 and fragments thereof, LN-421 and fragments thereof, and LN-121 and fragments thereof; such as the group consisting of LN-521 and fragments thereof, LN-511 and fragments thereof and LN-332 and fragments thereof; such as the group consisting of LN-521 and fragments thereof, and LN-511 and fragments thereof; such as the group consisting of LN-521 and fragments thereof or the group consisting of LN-511 and fragments thereof.
5. Method for the generation pancreatic islet-like cell aggregates in vitro according to item 3 or 4, wherein said laminins (LN) and fragments thereof are selected from the group consisting of LN-521, LN-511, LN-332, LN-421, LN-121 and LN-111; such as the group consisting of LN-521, LN-511, LN-332, LN-421 and LN-121, such as the group consisting of LN-521, LN-511 and LN-332; such as the group consisting of LN-521 and LN-511; such as wherein said laminins and fragments thereof are LN-521 or wherein said laminins and fragments thereof are LN-511.
6. Method for the generation pancreatic islet-like cell aggregates in vitro according to item 3 or 4, wherein said laminins (LN) and fragments comprise an E8 fragment of laminin; such as an E8 fragment of laminin selected from the group consisting of an E8 fragment of LN-511, an E8 fragment of LN-521, an E8 fragment of LN-332, an E8 fragment of LN-421, an E8 fragment of LN-121 and an E8 fragment of LN-111; such as the group consisting of an E8 fragment of LN-511, an E8 fragment of LN-521, an E8 fragment of LN-332, and E8 fragment of LN-421 and an E8 fragment of 121; such as the group consisting of an E8 fragment LN-511, an E8 fragment of LN-521 and an E8 fragment of LN-332; such as the group consisting of an E8 fragment of LN-511 and an E8 fragment of LN-521; such as an E8 fragment LN-511 or an E8 fragment of LN-521.
7. Method for the generation pancreatic islet-like cell aggregates in vitro according to any one of items 1-6, wherein in step i) more than approximately 15%, such as more than approximately 20%, such as more than approximately 25%, such as more than approximately 30%, such as more than approximately 35%, such as more than approximately 40%, such as more than approximately 45%, such as more than approximately 50% of the total cell population are EP cells characterized by the expression of NEUROD1;
   or in vitro wherein in step i) more than approximately 15%, such as more than approximately 20%, such as more than approximately 25%, such as more than approximately 30%, such as more than approximately 35%, such as more than approximately 40%, such as more than approximately 45%, such as more than approximately 50% of the total cell population are EP cells characterized by the expression of NKX6.1 and NEUROD1.
8. Method for the generation pancreatic islet-like cell aggregates in vitro according to any one of items 1-7, wherein step ii) of providing a single cell suspension of said population of EP cells is performed when more than approximately 15%, such as more than approximately 20%, such as more than approximately 25%, such as more than approximately 30%, such as more than approximately 35%, such as more than approximately 40%, such as more than approximately 45%, such as more than approximately 50% of the total cell population are EP cells characterized by the expression of NEUROD1;
   or wherein step ii) of providing a single cell suspension of said population of EP cells is performed when more than approximately 15%, such as more than approximately 20%, such as more than approximately 25%, such as more than approximately 30%, such as more than approximately 35%, such as more than approximately 40%, such as more than approximately 45%, such as more than approximately 50% of the total cell population are

EP cells characterized by the expression of NKX6.1 and NEUROD1.
9. in vitro in vitro Method for the generation pancreatic islet-like cell aggregates in vitro according to any one of items 1-8, wherein said pancreatic islet-like cell aggregates generated in step v) comprise approximately from 7 to 25%, such as from 7 to 20%, from 10 to 20%, such as from 15 to 20%, such as approximately 20% monohormonal α-cells
10. Method for the generation pancreatic islet-like cell aggregates in vitro according to any one of items 1-9, wherein said pancreatic islet-like cell aggregates generated in step v) comprise at most approximately 5%, such as at most approximately 4%, such as at most approximately 3%, such as at most approximately 2%, such as at most approximately 1%, such as at most approximately 0.5%, such as at most approximately 0.1%, proliferating cells, such as proliferating cells which express Ki-67.
11. Method for the generation pancreatic islet-like cell aggregates in vitro according to any one of items 1-10, wherein said wherein said pancreatic islet-like cell aggregates generated in step v) are scored after 38-42 days of culture.
12. Method for the generation pancreatic islet-like cell aggregates in vitro according to any one of items 1-11, wherein step ii) is performed prior to culturing the cells in a medium permissive of differentiation into pancreatic monohormonal β-cells.
13. Method for the generation pancreatic islet-like cell aggregates in vitro according to any one of items 1-12, wherein said step ii) is performed approximately within 6 days after the initiation of expression of NEUROD1 by said EP cells.
14. Method for the generation pancreatic islet-like cell aggregates in vitro according to any one of items 1-13, wherein said step ii) is performed within 1-5 days after the initiation of expression of NEUROD1 by said EP cells.
15. Method for the generation pancreatic islet-like cell aggregates in vitro according to any one of items 1-14, wherein said formation of 3D structures is step iii) is spontaneous formation of 3D structures.
16. Method for the generation pancreatic islet-like cell aggregates in vitro according to any one of items 1-14, wherein said formation of 3D structures is step iii) is forced or aided formation of 3D structures.
17. Method for the generation pancreatic islet-like cell aggregates in vitro according to any one of items 1-16, wherein step iv) comprises culturing EP for approximately 2 weeks or longer, such as approximately 3 weeks or longer, such as for approximately from 3 to 5 weeks, such as for approximately 4 weeks.
18. Method for the generation pancreatic islet-like cell aggregates in vitro according to any one of items 1-17, wherein the monohormonal β-cells generated in step v) have the ability to express, such as wherein the monohormonal β-cells generated in step v) express, insulin.
19. Method for the generation pancreatic islet-like cell aggregates in vitro according to any one of items 1-18, wherein the monohormonal β-cells generated in step v) have the ability to express, such as wherein the monohormonal β-cells generated in step v) express, C-peptide upon glucose stimulation.
20. Method for the generation pancreatic islet-like cell aggregates in vitro according to any one of items 1-19, wherein the monohormonal β-cells generated in step v) do not express glucagon and/or somatostatin.
21. Method for the generation pancreatic islet-like cell aggregates in vitro according to any one of items 1-20, wherein said pancreatic islet-like cell aggregates generated in step v) comprise at least 40% monohormonal β-cells; 7-25% monohormonal α-cells and less than 2% proliferating cells.
22. Method for the generation pancreatic islet-like cell aggregates in vitro according to any one of items 1-21, wherein prior to step i) the method comprises the steps a)-c) of
   a) providing a cell population of posterior foregut cells, such as posterior foregut cells characterized by expression of PDX1;
   b) culturing said cell population of posterior foregut cells for no more than approximately 78 hours, such as no more than approximately 72 hours, under conditions permissive of differentiation into pancreatic progenitor cells; and
   c) thereby generating a cell population of pancreatic progenitor cells, such as pancreatic progenitor cells characterized by expression of both PDX1 and NKX6.1.
23. Method for the generation pancreatic islet-like cell aggregates in vitro according to any one of items 1-22, wherein prior to step i) the method comprises the steps the steps a−1)-c−1) of:

a−1) providing a cell population of primitive gut tube cells, such as primitive gut tube cells characterized by expression of HNF1β and/or HNF4α;
b−1) culturing said cell population of primitive gut tube cells for no more than approximately 54 hours under conditions permissive of differentiation into posterior foregut cells; and c−1) thereby generating a population of posterior foregut cells, such as posterior foregut cells characterized by the expression of PDX1.
24. Method for the generation pancreatic islet-like cell aggregates in vitro according to any one of items 22-23, wherein said steps a−1)-c−1) are performed prior to said steps a)-c).
25. Method for the generation pancreatic islet-like cell aggregates in vitro according to any one of items 22-24, wherein prior to step i) the method comprises the steps a−1)-c−1) and the steps a) and c) of a−1) providing a cell population of primitive gut tube cells, such as primitive gut tube cells characterized by expression of HNF1β and/or HNF4α; b−1) culturing said cell population of primitive gut tube cells for no more than approximately 54 hours under conditions permissive of differentiation into posterior foregut cells;
   c−1) thereby generating a population of posterior foregut cells, such as posterior foregut cells characterized by the expression of PDX1.

a) providing the cell population of posterior foregut cells generated in step c−1), such as posterior foregut cells characterized by expression of PDX1;
b) culturing said cell population of posterior foregut cells for no more than approximately 78 hours, such as no more than approximately 72 hours, under conditions permissive of differentiation into pancreatic progenitor cells; and
c) thereby generating a cell population of pancreatic progenitor cells, such as pancreatic progenitor cells characterized by expression of both PDX1 and NKX6.1.
26. Method for the generation pancreatic islet-like cell aggregates in vitro according to any one of items 22-25, wherein further comprising, after the steps a)-c), the steps a+1)-c+1) of:
   a+1) providing a cell population of pancreatic progenitor cells generated in step c;
   b+1) culturing said cell population of pancreatic progenitor cells under conditions permissive of differentiation into endocrine progenitor cells; and
   c+1) thereby generating a population of endocrine progenitor cells, such as endocrine progenitor cells characterized by the expression of NEUROD1, such as by expression of NKX6.1 and NEUROD1.
27. Method for the generation pancreatic islet-like cell aggregates in vitro according to any one of items 22-26, wherein prior to step i) the method comprises the steps a−1) providing a cell population of primitive gut tube cells, such as primitive gut tube cells characterized by expression of HNF1β and/or HNF4α; b−1) culturing said cell population of primitive gut tube cells for no more than approximately 54 hours under conditions permissive of differentiation into posterior foregut cells;
   c−1) thereby generating a population of posterior foregut cells, such as posterior foregut cells characterized by the expression of PDX1;
   a) providing the cell population of posterior foregut cells generated in step c−1), such as posterior foregut cells characterized by expression of PDX1;
   b) culturing said cell population of posterior foregut cells for no more than approximately 78 hours, such as no more than approximately 72 hours, under conditions permissive of differentiation into pancreatic progenitor cells;
   c) thereby generating a cell population of pancreatic progenitor cells, such as pancreatic progenitor cells characterized by expression of both PDX1 and NKX6.1;
   a+1) providing the cell population of pancreatic progenitor cells generated in step c;
   b+1) culturing said cell population of pancreatic progenitor cells under conditions permissive of differentiation into endocrine progenitor cells; and
   c+1) thereby generating a population of endocrine progenitor cells, such as endocrine progenitor cells characterized by the expression of NEUROD1, such as by expression of NKX6.1 and NEUROD1.
28. Method for the generation pancreatic islet-like cell aggregates in vitro according to any one of items 22-27, wherein in step b) said cell population is cultured for a time period of approximately from 42 to 78 hours, such as a period of approximately from 44 to 76 hours, such as a period of approximately from 46 to 74 hours, such as a period of approximately from 48 to 72 hours.
29. Method for the generation pancreatic islet-like cell aggregates in vitro according to any one of items 22-28, wherein in step b) said cell population is cultured for a time period of approximately from 66 to 78 hours, such as a period of approximately from 68 to 76 hours, such as a period of approximately from 70 to 74 hours, such as a period of approximately 72 hours.
30. Method for the generation pancreatic islet-like cell aggregates in vitro according to any one of items 22-29, wherein in step b) said cell population is cultured for a time period of approximately from 42 to 54 hours, such as a period of approximately from 44 to 52 hours, such as a period of approximately from 46 to 50 hours, such as a period of approximately 48 hours.
31. Method for the generation pancreatic islet-like cell aggregates in vitro according to any one of items 22-30, wherein in step c) said cell population of pancreatic progenitor cells, such as pancreatic progenitor cells characterized by expression of both PDX1 and NKX6.1, is further characterized by expression of at least one marker selected from the group consisting of PTF1A, SOX9, HNF6 and CPA, such as a marker selected from the group consisting of PTF1A and SOX9.
32. Method for the generation pancreatic islet-like cell aggregates in vitro according to any one of items 22-31, wherein step b) comprises culturing said cell population in a culture medium in the presence of an effective amount of epidermal growth factor (EGF), such as human EGF, or a derivative or an agonist thereof; and an effective amount of nicotinamide (NIC) or a derivative or an agonist thereof.
33. Method for the generation pancreatic islet-like cell aggregates in vitro according to any one of items 22-32, wherein step b) comprises culturing said cell population in a culture medium in the presence of an effective amount of EGF, such as human EGF, and an effective amount of NIC.
34. Method for the generation pancreatic islet-like cell aggregates in vitro according to any one of items 32-33, wherein said effective amount of EGF or a derivative or agonist thereof is approximately from 50 to 200 ng/ml, such as approximately from 50 to 150 ng/ml, such as approximately from 75 to 125 ng/mL, such as approximately 100 ng/ml.
35. Method for the generation pancreatic islet-like cell aggregates in vitro according to any one of items 32-34, wherein said effective amount of NIC or a derivative or agonist thereof is approximately 5 to 20 mM, such as approximately from 5 to 15 mM, such as approximately from 8 to 12 mM, such as approximately 10 mM.
36. Method for the generation pancreatic islet-like cell aggregates in vitro according to any one of items 22-35, wherein step b) comprises culturing said cell population in a culture medium further comprising KGF, Activin A, retinoic acid, SANT-1, PDBu and LDN.
37. Method for the generation pancreatic islet-like cell aggregates in vitro according to any one of items 22-36, wherein said cells are cultured on a 2D substrate, such as wherein said cells are cultured adherent on a 2D substrate.
38. Method for the generation pancreatic islet-like cell aggregates in vitro according to item 37, wherein said 2D substrate comprises one or more components selected from the group consisting of laminins (LN) and fragments thereof, vitronectin and fragments thereof, fibronectin and fragments thereof, collagen and fragments thereof, gelatin and fragments thereof, functionalized silk (FN silk) and Matrigel™, such as one or more components selected from the group consisting of laminins (LN) and fragments thereof, vitronectin and fragments thereof, fibronectin and fragments thereof and Matrigel™.
39. Method for the generation pancreatic islet-like cell aggregates in vitro according to item 38, wherein said laminins (LN) and fragments thereof are selected from the group consisting of

LN-521 and fragments thereof, LN-511 and fragments thereof, LN-332 and fragments thereof, LN-421 and fragments thereof, LN-121 and fragments thereof and LN-111 and fragments thereof; such as the group consisting of LN-521 and fragments thereof, LN-511 and fragments thereof, LN-332 and fragments thereof, LN-421 and fragments thereof and LN-121 and fragments thereof;
such as the group consisting of LN-521 and fragments thereof, LN-511 and fragments thereof and LN-332 and fragments thereof;
such as the group consisting of LN-521 and fragments thereof or the group consisting of LN-511 and fragments thereof.
40. Method for the generation pancreatic islet-like cell aggregates in vitro according to any one of items 38-39, wherein said laminins (LN) and fragments thereof are selected from the group consisting of LN-521, LN-511, LN-332, LN-421, LN-121 and LN-111; such as the group consisting of LN-521, LN-511, LN-332, LN-421 and LN-121, such as the group consisting of LN-521, LN-511 and LN-332; such as the group consisting of

LN-521 and LN-511; such as wherein said laminins and fragments thereof are LN-521.
41. Method for the generation pancreatic islet-like cell aggregates in vitro according to any one of items 38-39, wherein said laminins (LN) and fragments comprise an E8 fragment of laminin; such as an E8 fragment of laminin selected from the group consisting of an E8 fragment of LN-511, an E8 fragment of LN-521, an E8 fragment of LN-332, an E8 fragment of LN-421, an E8 fragment of LN-121 and an E8 fragment of LN-111; such as the group consisting of an E8 fragment of LN-511, an E8 fragment of LN-521, an E8 fragment of LN-332, and E8 fragment of LN-421 and an E8 fragment of 121; such as the group consisting of an E8 fragment LN-511, an E8 fragment of LN-521 and an E8 fragment of LN-332; such as the group consisting of an E8 fragment of LN-511 and an E8 fragment of LN-521; such as an E8 fragment of LN-511 or an E8 fragment of LN-521.
42. Method for the generation pancreatic islet-like cell aggregates in vitro according to any one of items 22-41, wherein at least approximately 60%, such as at least approximately 55%, such as at least approximately 70%, such as at least approximately 75%, such as at least approximately 80% of the posterior foregut cells, such as posterior foregut cells characterized by expression of PDX1, in a) differentiate into pancreatic progenitor cells, such as pancreatic progenitor cells, characterized by expression of PDX1 and NKX6.1 in c).
43. Method for the generation pancreatic islet-like cell aggregates in vitro according to any one of items 22-42, wherein in step c) at least approximately 75%, such as at least approximately 80%, such as approximately from 80 to 85%, such as approximately from 80 to 90%, of the total cell population express PDX1.
44. Method for the generation pancreatic islet-like cell aggregates in vitro according to any one of items 22-43, wherein in step c) at most approximately 10% of the total cell population express NEUROD1.
45. Method for the generation pancreatic islet-like cell aggregates in vitro according to any one of items 22-44, wherein in step c) approximately from 40 to 70%, such as approximately from 30 to 50%, of the total cell population express NKX6.1.
46. Method for the generation pancreatic islet-like cell aggregates in vitro according to any one of items 23-45, wherein in step b−1) said cell population is cultured for no more than approximately 52 hours, such as no more than approximately 50 hours, such as no more than approximately 48 hours.
47. Method for the generation pancreatic islet-like cell aggregates in vitro according to any one of items 23-46, wherein in step b−1) said cell population is cultured for a time period of approximately from 18 to 54 hours, such as a period of approximately from 20 to 52 hours, such as a period of approximately from 22 to 50 hours, such as a period of approximately from 24 to 48 hours.
48. Method for the generation pancreatic islet-like cell aggregates in vitro according to any one of items 23-47, wherein in step b−1) said cell population is cultured for a time period of approximately from 42 to 54 hours, such as a period of approximately from 44 to 52 hours, such as a period of approximately from 46 to 50 hours, such as approximately 48 hours.
49. Method for the generation pancreatic islet-like cell aggregates in vitro according to any one of items 23-47, wherein in step b−1) said cell population is cultured for a time period of approximately from 18 to 30 hours, such as a period of approximately from 20 to 28 hours, such as a period of approximately from 22 to 26 hours, such as approximately 24 hours.
50. Method according to any one of items 23-49, wherein step b−1) comprises culturing said cell population in a culture medium comprising KGF, retinoic acid, SANT-1, PDBu and LDN.
51. Method for the generation pancreatic islet-like cell aggregates in vitro according to any one of items 26-50, wherein said population of endocrine progenitor cells, such as endocrine progenitor cells characterized by the expression of NEUROD1 or by the expression of NKX6.1 and NEUROD1, is further characterized by the expression of at least one of PDX1 and NGN3.
52. Method for the generation pancreatic islet-like cell aggregates in vitro according to any one of items 26-51, wherein in step b+1) said cell population of pancreatic progenitor cells is cultured for approximately from 3 to 5 days, such as approximately from 3 to 4 days or approximately 4 to 5 days, such as approximately 4 days or such as approximately 5 days.
53. Method for the generation pancreatic islet-like cell aggregates in vitro according to any one of items 26-52, wherein step b+1) comprises culturing said cell population in a culture medium comprising BTC, Alk5i II, GSI-XX, GC-1, LDN, retinoic acid and SANT-1.
54. Method for the generation pancreatic islet-like cell aggregates in vitro according to any one of items 26-53, wherein the said cells are cultured on a 2D substrate at least until the generation endocrine progenitor cells in step c+1).
55. Method for the generation pancreatic islet-like cell aggregates in vitro according to any one of items 26-54, wherein said 2D substrate comprises one or more components selected from the group consisting of laminins (LN) and fragments thereof, vitronectin and fragments thereof, fibronectin and fragments thereof, collagen and fragments thereof, gelatin and fragments thereof, functionalized silk (FN silk) and Matrigel™, such as one or more components selected from the group consisting of laminins (LN) and fragments thereof, vitronectin and fragments thereof, fibronectin and fragments thereof and Matrigel™ 56. Method for the generation pancreatic islet-like cell aggregates in vitro according to any one of items 26-55, wherein said laminins (LN) and fragments thereof are selected from the group consisting of LN-521 and fragments thereof, LN-511 and fragments thereof, LN-332 and fragments thereof, LN-421 and fragments thereof, LN-121 and fragments thereof and LN-111 and fragments thereof;
   such as the group consisting of LN-521 and fragments thereof, LN-511 and fragments thereof, LN-332 and fragments thereof, LN-421 and fragments thereof and LN-121 and fragments thereof;
   such as the group consisting of LN-521 and fragments thereof, LN-511 and fragments thereof and LN-332 and fragments thereof;
   such as the group consisting of LN-521 and fragments thereof and LN-511 and fragments thereof.

such as the group consisting of LN-521 and fragments thereof or the group consisting of LN-511 and fragments thereof.
57. Method for the generation pancreatic islet-like cell aggregates in vitro according to any one of items 26-56, wherein said laminins (LN) and fragments thereof are selected from the group consisting of LN-521, LN-511, LN-332, LN-421, LN-121 and LN-111; such as the group consisting of LN-521, LN-511, LN-332, LN-421 and LN-121, such as the group consisting of LN-521, LN-511 and LN-332; such as the group consisting of LN-521 and LN-511; such as wherein said laminins and fragments thereof are LN-521 or wherein said laminins and fragments thereof are LN-511.
58. Method for the generation pancreatic islet-like cell aggregates in vitro according to any one of items 26-56, wherein said laminins (LN) and fragments comprise an E8 fragment of laminin, such as an E8 fragment of laminin selected from the group consisting of an E8 fragment of LN-511, an E8 fragment of LN-521, an E8 fragment of LN-332, an E8 fragment of LN-421, an E8 fragment of LN-121 and an E8 fragment of LN-111; such as the group consisting of an E8 fragment of LN-511, an E8 fragment of LN-521, an E8 fragment of LN-332, and E8 fragment of LN-421 and an E8 fragment of 121; such as the group consisting of an E8 fragment LN-511, an E8 fragment of LN-521 and an E8 fragment of LN-332; such as the group consisting of an E8 fragment of LN-511 and an E8 fragment of LN-521;
   such as an E8 fragment LN-511 or an E8 fragment of LN-521.
59. Method for the generation pancreatic islet-like cell aggregates in vitro according to any one of items 26-58, wherein in step c+1) more than approximately 30%, such as more than approximately 40%, such as more than approximately 45%, such as more than approximately 50% of the total cell population are endocrine progenitor cells, such as endocrine progenitor cells characterized by the expression of NKX6.1 and NEUROD1.
60. Method for the generation pancreatic islet-like cell aggregates in vitro according to any one of items 26-58, wherein the number of endocrine progenitor cells in step c+1) is higher compared to the number of endocrine cells obtained using the corresponding method in which step b) of culturing said cell population of posterior foregut cells under conditions permissive of differentiation into pancreatic progenitor cells is for approximately 24 hours or less and/or is for approximately 96 hours or more.
61. Method for the generation pancreatic islet-like cell aggregates in vitro according to any one of items 26-60, wherein said method results in at least approximately 10%, such as at least approximately 15%, such as at least approximately 20%, such as at least approximately 30%, such as at least approximately 40%, such as at least 50%, such as at least 60% more endocrine progenitor cells than the corresponding method in which step b) of culturing said cell population of posterior foregut cells under conditions permissive of differentiation into pancreatic progenitor cells is for approximately 24 hours or less and/or is for approximately 96 hours or more.
62. Method for the generation pancreatic islet-like cell aggregates in vitro according to any one of items 26-61, further comprising culturing said endocrine progenitor cells in conditions allowing for differentiation into monohormonal pancreatic β-cells.
63. Method for the generation pancreatic islet-like cell aggregates in vitro according to any one of items 26-62, wherein the cells are cultured on a 2D substrate, such as adherent on a 2D substrate during steps a−1) to c+1).
64. Method for the generation pancreatic islet-like cell aggregates in vitro according to any one of items 26-63, wherein the cells are not transferred from culture on a 2D substrate to culture on a 3D substrate prior to exhibiting expression of markers characteristic of endocrine progenitor cells, such as wherein the cells are not transferred from adherent culture on a 2D substrate to culture on a 3D substrate prior to exhibiting expression of markers characteristic of endocrine progenitor cells.
65. Method for the generation pancreatic islet-like cell aggregates in vitro according to any one of items 1-64, wherein the EP cell population in step i) is derived from a culture of pluripotent stem cells, such as a culture of induced pluripotent stem cells or a culture of embryonic stem cells, such as a culture of human induced pluripotent stem cells or a culture of human embryonic stem cells.
66. Method for the generation pancreatic islet-like cell aggregates in vitro according to any one of items 22-65, wherein the cell population provided in step a−1), a) or a+1) is derived from a culture of pluripotent stem cells, such as a culture of induced pluripotent stem cells or a culture of embryonic stem cells, such as a culture of human induced pluripotent stem cells or a culture of human embryonic stem cells.
67. Method for the generation pancreatic islet-like cell aggregates in vitro according to any one of items 65-66, wherein the cell population provided in is derived from a culture of human embryonic stem cells.
68. Method for the generation pancreatic islet-like cell aggregates in vitro according to any one of items 65-67, wherein said cell population is a mammalian cell population, such as human cell population.
69. Method for the generation pancreatic islet-like cell aggregates in vitro according to any one of items 65-68, wherein said cell population is derived from a human embryonic stem cell line selected from the group of embryonic stem cell lines consisting of HS980 cells, H1 cells and H9 cells, such as the group of embryonic stem cell lines consisting of HS980 cells and H1 cells, or the group of embryonic stem cell lines consisting of H1 and H9 cells, or the group of embryonic stem cell lines consisting of HS980 cells and H9 cells.
70. Method for the generation pancreatic islet-like cell aggregates in vitro according to any one of items 65-68, wherein said cell population is derived from a human induced pluripotent stem cell population.
71. Method for the generation pancreatic islet-like cell aggregates in vitro according to any one of items 1-70, further comprising cryopreservation of

EP cells prior to step i).
72. Isolated pancreatic islet-like cell aggregates obtainable by the method according to any one of the proceeding items.
73. Isolated population of pancreatic islet-like cell aggregates obtainable by the method according to any one of the proceeding items.
74. Isolated pancreatic islet-like cell aggregates according to item 72 or isolated population of pancreatic islet-like cell aggregates according to item 73, wherein said pancreatic islet-like cell aggregates comprise more than approximately 40%, such as approximately from 40 to 70%, such as approximately from 40 to 60%, such as approximately from 40 to 50% of the total cell population are monohormonal β-cells, such as monohormonal β-cells characterized by the expression of insulin.
75. Isolated pancreatic islet-like cell aggregates according to any one of items 72-74 or isolated population of pancreatic islet-like cell aggregates according to any one of items 73-74, wherein the cells comprising said islet-like cell aggregates have not been subject to enrichment for a desired phenotype, such as have not been subject to enrichment by manual or automated intervention, such wherein said cells have not been subject to enrichment prior to forming the 3D structures in step iii).
76. Isolated pancreatic islet-like cell aggregates according to any one of items 72-75 or isolated population of pancreatic islet-like cell aggregates according to any one of items 73-75, wherein the cells comprising said islet-like aggregates have not been subject sorting for a desired phenotype based on marker expression, such wherein said cells have not been subject to sorting prior to forming the 3D structures in step iii).
77. Isolated pancreatic islet-like cell aggregates according to any one of items 72-76 or isolated population of pancreatic islet-like cell aggregates according to any one of items 73-76, wherein the cells prior to forming comprising said islet-like cell aggregates have not been subject sorting for a desired phenotype based on FACS, such wherein said cells have not been subject to sorting prior to forming the 3D structures in step iii).
78. Isolated pancreatic islet-like cell aggregates according to any one of items 72-77 or isolated population of pancreatic islet-like cell aggregates according to any one of items 73-77, wherein said pancreatic islet-like cell aggregates comprise 7 to 25%, such as from 7 to 20%, from 10 to 20%, such as from 15 to 20%, such as approximately 20% monohormonal α-cells.
79. Isolated pancreatic islet-like cell aggregates according to any one of items 72-78 or isolated population of pancreatic islet-like cell aggregates according to any one of items 73-78, wherein said pancreatic islet-like cell aggregates comprise at least 40%, such as at least 50% monohormonal β-cells; approximately from 15 to 20%, such as approximately 20% monohormonal α-cells and less than approximately 2%, such as less than approximately 1%, proliferating cells.
80. Cells obtained from at least one isolated pancreatic islet as defined in any one of items 72-79, such as obtained by dissociation of said pancreatic islet-like cell aggregates.
81. Isolated pancreatic islet-like cell aggregates according to any one of items 72-79 or isolated population of pancreatic islet-like cell aggregates according to any one of items 73-79 or cells according to item 80, for use in therapy.
82. Isolated pancreatic islet-like cell aggregates according to any one of items 72-79 and 81 or isolated population of pancreatic islet-like cell aggregates according to any one of items 73-79 and 81 or cells according to item 80 or 81, for use in the treatment, prevention and/or amelioration of diabetes, such as type 1 or type 2 diabetes.
83. Isolated pancreatic islet-like cell aggregates according to any one of items 72-79 or isolated population of pancreatic islet-like cell aggregates according to any one of items 73-79, for use in the treatment in therapy, wherein said islet-like cell aggregates or cells have been generated by a method according to any one of items 1-71.
84. Isolated pancreatic islet-like cell aggregates according to any one of items 72-79 and 81 or isolated population of pancreatic islet-like cell aggregates according to any one of items 73-79 and 81 or cells according to item 80 or 81 for use in the treatment, prevention and/or amelioration of diabetes, such as type 1 or type 2 diabetes, wherein said islet-like cell aggregates or cells have been generated by a method according to any one of items 1-71.
85. Isolated pancreatic islet-like cell aggregates according to any one of items 72-79 and 81 or isolated population of pancreatic islet-like cell aggregates according to any one of items 73-79 and 81 or cells according to item 80 or 81 for use in the treatment in therapy, wherein said use comprises the steps of generating isolated pancreatic islet-like cell aggregates, according to the method as defined in any one of items 1-71; and administering a therapeutically effective amount of said islet-like cell aggregates to a patient or wherein said use comprises the steps of generating isolated pancreatic islet-like cell aggregates, according to the method as defined in any one of items 1-71;
   dissociating the pancreatic islet-like cell aggregates, and administering a therapeutically effective amount of said dissociated islet cells to a patient.
86. Isolated pancreatic islet-like cell aggregates according to any one of items 72-79 and 81 or isolated population of pancreatic islet-like cell aggregates according to any one of items 73-79 and 81 or cells according to item 80 or 81 for use in the treatment, prevention and/or amelioration of diabetes, such as type 1 or type 2 diabetes, wherein said use comprises the steps of generating isolated pancreatic islet-like cell aggregates, according to the method as defined in any one of items 1-71; and administering a therapeutically effective amount of said cells to a patient or wherein said use comprises the steps of generating isolated pancreatic islet-like cell aggregates, according to the method as defined in any one of items 1-71;
   dissociating the pancreatic islet-like cell aggregates, and administering therapeutically effective amount of said dissociated islet cells to a patient.
87. Isolated pancreatic islet-like cell aggregates according to any one of items 72-79 and 81 or isolated population of pancreatic islet-like cell aggregates according to any one of items 73-79 and 81 or cells according to item 80 or 81 to 87, wherein said use comprises transplantation of said islet-like cell aggregates or cells into a patient in need thereof.
88. Pharmaceutical composition comprising Isolated pancreatic islet-like cell aggregates according to any one of items 72-79 and 81 or isolated population of pancreatic islet-like cell aggregates according to any one of items 73-79 and 81 or cells according to item 80 or 81, and at least one pharmaceutically acceptable excipient or carrier.
89. Kit of parts comprising Isolated pancreatic islet-like cell aggregates according to any one of items 72-79 and 81 or isolated population of pancreatic islet-like cell aggregates according to any one of items 73-79 and 81 or cells according to item 80 or 81 or a pharmaceutical composition according to item 88 and a suitable carrier substrate.
90. Kit of parts according to item 89, wherein said suitable carrier substrate is a 3D substrate.
91. Use of isolated pancreatic islet-like cell aggregates according to any one of items 72-79 and 81 or isolated population of pancreatic islet-like cell aggregates according to any one of items 73-79 and 81 or cells according to item 80 or 81 in drug screening, such as in vitro drug screening.
92. Method of in vitro drug screening, comprising the steps of generating isolated pancreatic islet-like aggregates, according to the method as defined in any one of items 1-71; and exposing said islet-like cell aggregates to at least one candidate drug compound.
93. Method of in vitro drug screening according to item 92, comprising the steps of generating isolated pancreatic islet-like aggregates, according to the method as defined in any one of items 1-71;
   dissociating the islet-like cell aggregates; and
   exposing at least a fraction of the dissociated islet cells to at least one candidate drug compound.
94. Method of treatment of a patient in need thereof, comprising administering to said patient a therapeutically effective amount of isolated pancreatic islet-like cell aggregates according to any one of items 72-79 and 81 or isolated population of pancreatic islet-like cell aggregates according to any one of items 73-79 and 81 or cells according to item 80 or 81 . . .
95. Method of treatment of a patient in need thereof, such as method of treatment of diabetes in a patient in need thereof, comprising the steps of generating isolated pancreatic islet-like cell aggregates, according to the method as defined in any one of items 1-71; and administering to said patient a therapeutically effective amount of said islet-like cell aggregates or generating isolated pancreatic islet-like cell aggregates, according to the method as defined in any one of items 1-71;
   dissociating the pancreatic islet-like cell aggregates; and administering therapeutically effective amount of said dissociated islet cells to said patient.
96. Method of treatment of diabetes in a patient in need thereof, comprising administering to said patient a therapeutically effective amount of isolated pancreatic islet-like cell aggregates according to any one of items 72-79 and 81 or isolated population of pancreatic islet-like cell aggregates according to any one of items 73-79 and 81 or cells according to item 80 or 81.
97. Method of treatment of diabetes in a patient in need thereof according to item 95 or 96, wherein said patient is suffering from type 1 or type 2 diabetes.
98. Method of treatment of diabetes in a patient in need thereof according to any one of items 94 to 97, wherein said administration comprises transplantation of said islet-like cell aggregates or cells into said patient.
99. Use of isolated pancreatic islet-like cell aggregates according to any one of items 72-79 and 81 or isolated population of pancreatic islet-like cell aggregates according to any one of items 73-79 and 81 or cells according to item 80 or 81 for the manufacture of a medicament for the treatment of diabetes in a patient in need thereof.
100. Use according to item 99, wherein said manufacture of said medicament comprises generation of pancreatic islet-like aggregates is by a method as defined in any one of items 1-71, and optionally dissociation thereof.
101. Method for the generation pancreatic islet-like cell aggregates in vitro, comprising the steps of:
   a−1) providing a cell population of primitive gut tube cells, such as primitive gut tube cells characterized by expression of HNF1β and/or HNF4α;
      b−1) culturing said cell population of primitive gut tube cells for no more than approximately 54 hours under conditions permissive of differentiation into posterior foregut cells;
   c−1) thereby generating a population of posterior foregut cells, such as posterior foregut cells characterized by the expression of PDX1.
   a) providing the cell population of posterior foregut cells generated in step c−1), such as posterior foregut cells characterized by expression of PDX1;
   b) culturing said cell population of posterior foregut cells for no more than approximately 78 hours, such as no more than approximately 72 hours, under conditions permissive of differentiation into pancreatic progenitor cells;
   c) thereby generating a cell population of pancreatic progenitor cells, such as pancreatic progenitor cells characterized by expression of both PDX1 and NKX6.1;
   a+1) providing the cell population of pancreatic progenitor cells generated in step c);
   b+1) culturing said cell population of pancreatic progenitor cells under conditions permissive of differentiation into endocrine progenitor cells;
   c+1) thereby generating a population of endocrine progenitor cells, such as endocrine progenitor cells characterized by the expression of NEUROD1, such as by expression of NKX6.1 and NEUROD1;
   i) providing the population of endocrine progenitor (EP) cells generated in step c+1), such as EP cells characterized by the expression of NEUROD1; such as EP cells characterized by the expression of NKX6.1 and NEUROD1;
   ii) providing a single cell suspension of said population of EP cells;
   iii) allowing said population of EP cells in single cell suspension to form 3D structures;
   iv) culturing said population of EP cell in the form of 3D structures in 3D culture conditions permissive of differentiation pancreatic monohormonal β-cells to provide pancreatic islet-like cell aggregates; and
   v) thereby generating pancreatic islet-like cell aggregatesl, wherein said pancreatic islet-like cell aggregates comprise at least approximately 25% monohormonal β-cells; or
   v) thereby generating a population of pancreatic islet-like cell aggregates comprising monohormonal β-cells, wherein said population comprises pancreatic islet-like cell aggregates comprises at least approximately 25% monohormonal β-cells.

1. Method for the generation of pancreatic islet-like cell aggregates in vitro, comprising the steps of
i) providing a population comprising endocrine progenitor (EP) cells, characterized by the expression of NKX6.1 and NEUROD1 , wherein the population is an adherent culture of EP cells on a 2D substrate;
ii) providing a single cell suspension of said population of EP cells;
iii) allowing said population of EP cells in single cell suspension to form 3D structures;
iv) culturing said population of EP cells in the form of 3D structures in 3D culture conditions permissive of differentiation into pancreatic monohormonal β-cells to provide pancreatic islet-like cell aggregates; and
v) thereby generating pancreatic islet-like cell aggregates comprising monohormonal β-cells,
wherein said pancreatic islet-like cell aggregates comprise at most approximately 2% proliferating cells and wherein said pancreatic islet-like cell aggregates comprise at least approximately 40% monohormonal β-cells.

2. Method for the generation of pancreatic islet-like cell aggregates in vitro according to claim 1, wherein said 2D substrate comprises one or more components selected from the group consisting of laminins (LN) and fragments thereof, vitronectin and fragments thereof, fibronectin and fragments thereof, collagen and fragments thereof, gelatin and fragments thereof, functionalized silk (FN silk) and Matrigel.

3-5. (canceled)

6. Method for the generation of pancreatic islet-like cell aggregates in vitro according to claim 1, wherein said pancreatic islet-like cell aggregates generated in step v) comprise approximately from 7 to 25% monohormonal α-cells.

7. Method for the generation of pancreatic islet-like cell aggregates in vitro according to claim 1, wherein said pancreatic islet-like cell aggregates generated in step v) comprise at most approximately 1% proliferating cells.

8. (canceled)

9. Method for the generation of pancreatic islet-like cell aggregates in vitro according to claim 1, wherein step ii) is performed prior to culturing the cells in a medium permissive of differentiation into pancreatic monohormonal β-cells.

10. Method for the generation of pancreatic islet-like cell aggregates in vitro according to claim 1, wherein said formation of 3D structures in step iii) is spontaneous formation of 3D structures or wherein said formation of 3D structures in step iii) is forced or aided formation of 3D structures.

11. (canceled)

12. Method for the generation of pancreatic islet-like cell aggregates in vitro according to claim 1, wherein step iv) comprises culturing EP for approximately 2 weeks or longer.

13. (canceled)

14. Method for the generation of pancreatic islet-like cell aggregates in vitro according to claim 1, wherein the monohormonal β-cells generated in step v) have the ability to express C-peptide upon glucose stimulation.

15. (canceled)

16. Method for the generation of pancreatic islet-like cell aggregates in vitro according to claim 1, wherein said pancreatic islet-like cell aggregates generated in step v) comprise at least 40% monohormonal β-cells; 7-25% monohormonal α-cells and less than 2% proliferating cells.

17. Method for the generation of pancreatic islet-like cell aggregates in vitro according to claim 1, wherein prior to step i) the method comprises the steps a−1)-c−1) and the steps a) and c) of
a−1) providing a cell population of primitive gut tube cells characterized by expression of HNF1β and/or HNF4α;
b−1) culturing said cell population of primitive gut tube cells for no more than approximately 54 hours under conditions permissive of differentiation into posterior foregut cells;
c−1) thereby generating a population of posterior foregut cells characterized by the expression of PDX1.
a) providing the cell population of posterior foregut cells generated in step c−1) characterized by expression of PDX1;
b) culturing said cell population of posterior foregut cells for no more than approximately 78 hours under conditions permissive of differentiation into pancreatic progenitor cells; and
c) thereby generating a cell population of pancreatic progenitor cells characterized by expression of both PDX1 and NKX6.1.

18. Method for the generation of pancreatic islet-like cell aggregates in vitro according to claim 17, wherein further comprising, after the steps a)-c), the steps a+1)-c+1) of:
a+1) providing a cell population of pancreatic progenitor cells generated in step c;
b+1) culturing said cell population of pancreatic progenitor cells under conditions permissive of differentiation into endocrine progenitor cells; and
c+1) thereby generating a population of endocrine progenitor cells characterized by expression of NKX6.1 and NEUROD1.

19-22. (canceled)

23. Method for the generation of pancreatic islet-like cell aggregates in vitro according to claim 18, wherein in step b+1) said cell population of pancreatic progenitor cells is cultured for approximately from 3 to 5 days.

24. Method for the generation of pancreatic islet-like cell aggregates in vitro according to claim 18 wherein the cells are cultured on a 2D substrate during steps a−1) to c+1).

25. Method for the generation of pancreatic islet-like cell aggregates in vitro according to claim 1, wherein the cells are not transferred from culture on a 2D substrate to culture on a 3D substrate prior to exhibiting expression of markers characteristic of endocrine progenitor cells.

26. Method for the generation of pancreatic islet-like cell aggregates in vitro according to claim 1, wherein the EP cell population in step i) is derived from a culture of pluripotent stem cells.

27-30. (canceled)

31. Isolated pancreatic islet-like cell aggregates or population of pancreatic islet-like cell aggregates obtainable by the method according to claim 1, wherein said pancreatic islet-like cell aggregates comprise at least 40% monohormonal β-cells; approximately from 15 to 20% monohormonal α-cells and less than approximately 2% proliferating cells.

32. (canceled)

33. Isolated pancreatic islet-like cell aggregates or isolated population of pancreatic islet-like cell aggregates according to claim 31, wherein approximately from 40 to 50% of the total cell population are monohormonal β-cells characterized by the expression of insulin.

34-36. (canceled)

38. Isolated pancreatic islet-like cell aggregates or isolated population of pancreatic islet-like cell aggregates according to claim 31, wherein said pancreatic islet-like cell aggregates comprise from 7 to 20monohormonal α-cells.

38-47. (canceled)

48. Method of treatment of diabetes a patient in need thereof, comprising administering to said patient a therapeutically effective amount of the isolated pancreatic islet-like cell aggregates or isolated population of pancreatic islet-like cell aggregates according to claim 31.

49. Method of treatment of diabetes in a patient in need thereof, comprising the steps of generating isolated pancreatic islet-like cell aggregates according to the method as defined in claim 1; and
administering to said patient a therapeutically effective amount of said islet-like cell aggregates.

50. (canceled)


SEQUENCE LISTING (16 sequences)

SEQ ID NO: 1 - protein, 5 aa, synthetic construct
IKVAV

SEQ ID NO: 2 - protein, 5 aa, synthetic construct
YIGSR

SEQ ID NO: 3 - protein, 5 aa, synthetic construct
EPDIM

SEQ ID NO: 4 - protein, 5 aa, synthetic construct
NKDIL

SEQ ID NO: 5 - protein, 5 aa, synthetic construct
GRKRK

SEQ ID NO: 6 - protein, 15 aa, synthetic construct
KYGAASIKVAVSADR

SEQ ID NO: 7 - protein, 12 aa, synthetic construct
NGEPRGDTYRAY

SEQ ID NO: 8 - protein, 11 aa, synthetic construct
PQVTRGDVFTM

SEQ ID NO: 9 - protein, 12 aa, synthetic construct
AVTGRGDSPASS

SEQ ID NO: 10 - protein, 8 aa, synthetic construct
TGRGDSPA

SEQ ID NO: 11 - protein, 10 aa, synthetic construct
CTGRGDSPAC

SEQ ID NO: 12 - protein, 10 aa, synthetic construct
  VARIANT 2: note = X=A or R or N or D or Q or E or G or H or I or L or K or M or F or P or S or T or W or Y or V
  VARIANT 3: note = X=A or R or N or D or Q or E or G or H or I or L or K or M or F or P or S or T or W or Y or V
  VARIANT 7: note = X=A or R or N or D or Q or E or G or H or I or L or K or M or F or P or S or T or W or Y or V
  VARIANT 8: note = X=A or R or N or D or Q or E or G or H or I or L or K or M or F or P or S or T or W or Y or V
  VARIANT 9: note = X=A or R or N or D or Q or E or G or H or I or L or K or M or F or P or S or T or W or Y or V
  DISULFID 1..10: note = Cysteine in position one is connected to Cysteine in position 10 via a disulphide bond
CXXRGDXXXC

SEQ ID NO: 13 - protein, 98 aa, Euprosthenops australis
SRLSSPSAVSRVSSAVSSLVSNGQVNMAALPNIISNISSSVSASAPGASGCEVIVQALLEVITALVQIVSSSSVGYINPSAVNQITNVVANAMAQVMG

SEQ ID NO: 14 - protein, 277 aa, synthetic construct
GPNSCTGRGDSPACGSASGQGGYGGLGQGGYGQGAGSSAAAAAAAAAAAAGGQGGQGQGGYGQGSGGSAAAAAAAAAAAAAAAGRGQGGYGQGSGGNAAAAAAAAAAAAAAAGQGGQGGYGRQSQGAGSAAAAAAAAAAAAAAGSGQGGYGGQGQGGYGQSSASASAAASAASTVANSVSRLSSPSAVSRVSSAVSSLVSNGQVNMAALPNIISNISSSVSASAPGASGCEVIVQALLEVITALVQIVSSSSVGYINPSAVNQITNVVANAMAQVMG

SEQ ID NO: 15 - protein, 271 aa, synthetic construct
GPNSRGDAGAASGQGGYGGLGQGGYGQGAGSSAAAAAAAAAAAAGGQGGQGQGGYGQGSGGSAAAAAAAAAAAAAAAGRGQGGYGQGSGGNAAAAAAAAAAAAAAAGQGGQGGYGRQSQGAGSAAAAAAAAAAAAAAGSGQGGYGGQGQGGYGQSSASASAAASAASTVANSVSRLSSPSAVSRVSSAVSSLVSNGQVNMAALPNIISNISSSVSASAPGASGCEVIVQALLEVITALVQIVSSSSVGYINPSAVNQITNVVANAMAQVMG

SEQ ID NO: 16 - protein, 263 aa, synthetic construct
GPNSGQGGYGGLGQGGYGQGAGSSAAAAAAAAAAAAGGQGGQGQGGYGQGSGGSAAAAAAAAAAAAAAAGRGQGGYGQGSGGNAAAAAAAAAAAAAAAGQGGQGGYGRQSQGAGSAAAAAAAAAAAAAAGSGQGGYGGQGQGGYGQSSASASAAASAASTVANSVSRLSSPSAVSRVSSAVSSLVSNGQVNMAALPNIISNISSSVSASAPGASGCEVIVQALLEVITALVQIVSSSSVGYINPSAVNQITNVVANAMAQVMG